(19) United States
(12) Patent Application Publication
(10) Pub. No.: US 2007/0254305 A1
(43) Pub. Date: Nov. 1, 2007

(54) Methods of whole genome or microarray expression profiling using nucleic acids prepared from formalin fixed paraffin embedded tissue

(75) Inventors: Soonmyung Paik, Pittsburgh, PA (US); Katherine Lea Pogue-Geile, Pittsburgh, PA (US); Chungyeul Kim, Wexford, PA (US)

Correspondence Address: VINSON & ELKINS L.L.P., 1001 FANNIN STREET, 2300 FIRST CITY TOWER, HOUSTON, TX, 77002-6760, US

(73) Assignee: NSABP Foundation, Inc., Pittsburgh, PA (US)

(21) Appl. No.: 11/796,752

(22) Filed: Apr. 30, 2007

Related U.S. Application Data
(60) Provisional application No. 60/796,260, filed on Apr. 28, 2006.

(51) Int. Cl. C12Q 1/68

(52) U.S. Cl. 435/6

(57) ABSTRACT

The present invention provides novel methods for analyzing gene expression levels from fresh or aged (more than one year old) formalin-fixed, paraffin-embedded tissue (“FFPET”) samples that comprise pre-hybridizing a labeled nucleic acid sample prepared from the formalin-fixed, paraffin-embedded tissue sample with a first microarray, hybridizing the unbound labeled nucleic acid sample with a second microarray, and detecting the labeled nucleic acid sample bound to the second microarray. The pre-hybridization step results in an increase in the specific gene signals in subsequent hybridizations with high density gene expression arrays. The first microarray used for the pre-hybridization step can be either a new or used microarray. Importantly, from a cost-savings perspective, the inventors determined that when the first microarray used for the pre-hybridization step is a previously used microarray, the results of the subsequent hybridization on a second microarray are nearly identical to the results obtained when the pre-hybridization was carried out using a new or previously unused microarray.

### CROSS-REFERENCE TO RELATED APPLICATION

This application claims the benefit of U.S. Provisional Patent Application Ser. Number 60/796,260, filed Apr. 28, 2006, which is incorporated herein by reference in its entirety.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### INCORPORATION-BY-REFERENCE OF MATERIAL SUBMITTED ON A COMPACT DISC

Not Applicable.

### BACKGROUND OF THE INVENTION

1. Field of the Invention

The present disclosure relates to methods for analyzing gene expression levels from fresh or aged formalin-fixed, paraffin-embedded tissue samples.

2. Description of the Related Art

The use of gene expression profiling is not only prevalent in various research applications, but is rapidly becoming part of many therapeutic regimes. For example, in cancer research and treatment, it is often advantageous to examine gene expression levels in samples that represent many stages of tumor advancement, and patients representing a wide variety of demographics, as well as multiple other variables. One potentially exceptional source for this type of information comes in the form of formalin-fixed, paraffin-embedded tissue (“FFPET”) samples, which are routinely created from biopsy specimens taken from patients undergoing a variety of therapeutic regimens for a variety of different diseases, and are usually associated with the corresponding clinical records. For example, tumor biopsy FFPET samples are often linked with cancer stage classification, patient survival, and treatment regime, thereby providing a potential wealth of information that can be cross-referenced and correlated with gene expression patterns. However, the poor quality and quantity of nucleic acids isolated from FFPET samples has led to their underutilization in gene expression profiling studies.

It has long been known that RNA can be purified and analyzed from FFPET samples (Rupp and Locker, Biotechniques 6:56-60, 1988). Although RNA isolated from FFPET samples is moderately to highly degraded and fragmented, techniques were developed for isolating RNA from FFPET samples that was suitable for analysis by reverse transcription polymerase chain reaction (“RT-PCR”; Stanta and Schneider, Biotechniques 11:304-308, 1991, Finke et al., Biotechniques 14:448-453, 1993). In addition to being degraded and fragmented, chemical modification of RNA by formalin restricts the binding of oligo-dT primers to the polyadenylic acid tail and impedes the efficiency of reverse transcription. Heating in high-pH Tris buffer can partially reverse the modification and allow the reverse transcription to proceed. Therefore, for relatively fresh paraffin blocks with high molecular weight RNA preserved in the specimen, usual method of cDNA synthesis can be applied. Initial attempts to quantitatively analyze RNA isolated from FFPET samples involved techniques such as dot blot hybridization or capillary electrophoresis (Stanta and Bonin, Biotechniques 24:271-276, 1998), which are not amenable to the analysis of large numbers of samples.

More recently, techniques were developed to analyze gene expression information from FFPET samples using quantitative RT-PCR (“qRT-PCR”; Godfrey et al., J. Mol. Diagn. 2:84-91, 2000, Specht et al., Am. J. Pathol. 158:419-429, 2001, Abrahamsen et al., J. Mol. Diagn. 5:34-41, 2003). These real-time assays allow for interrogation of the expression level of one gene at a time, but with great accuracy and a wide dynamic range. However gene-specific priming is required for cDNA synthesis for each gene target because oligo-dT primed reverse transcription is not feasible with the fragmented and chemically modified RNA. This means that the assay for each gene has to be done in a separate reaction tube from the point of cDNA synthesis onward. Therefore, robotic assisted pipetting is usually used to ensure highly accurate quantitative pipetting in order to obtain reproducible assay results. For this reason, when more than a handful of genes are to be assayed, fairly sophisticated laboratory facilities are required. Furthermore, the number of genes that can be interrogated in a single qRT-PCR experiment is limited (typically around 70 genes/2 days/sample or 1 gene/2 days/70 samples). Even with extensive automation, perhaps 200 genes in a single sample could be interrogated using qRT-PCR in one experiment. Additionally, qRT-PCR requires a relatively large quantity of RNA, on the order of 30 genes/μg of RNA, and is quite labor and material intensive. In addition, at least one study has shown that the absolute signal decreases significantly if the paraffin blocks have been stored for a long time, resulting in 100-fold reduction in signal if the paraffin block is 10 years old compared with freshly produced block (Cronin et al., Am. J Pathol. 164:35-42, 2004), but careful normalization based on genes with minimal variation of expression level among different tumor samples can largely compensate for these differences in absolute signal.

The development of microarray based analyses to interrogate gene expression profiles has allowed large numbers of genes to be analyzed with less labor and materials, and would appear to be ideally suited for the analysis of FFPET samples. Unfortunately, the use of microarray based assays to interrogate gene expression profiles in FFPET samples has been of limited usefulness. Recent studies using microarray analysis of FFPET samples concluded FFPET tissues did not yield reproducible gene expression data (Karsten et al., Nucleic Acids Res. 30:e4, 2002), and another study suggested that chemical modification and fragmentation of mRNA extracted from FFPET is a barrier to applying known methods of generating labeled probes that are suitable for whole genome expression profiling in microarray based assays (Paik, Clin. Cancer Res. 12:1019S-1023S, 2006).

Recently, a method for obtaining gene expression information specifically developed for use with FFPET samples (Bibikova et al., Am. J Pathol., 165:1799-1807, 2004) was developed by Illumina, Incorporated (San Diego, Calif.). This method is referred to as cDNA-mediated annealing, selection, extension, and ligation (“DASL”), and is based on a bead array platform. DASL is reportedly useful for analyzing FFPET samples that have been stored for up to 12 years (Illumina, Incorporated, and Bibikova, supra). The DASL assay monitors gene expression by targeting sequences in cDNAs with sets of query oligonucleotides composed of multiple parts. In addition to gene-specific sequences, the query oligonucleotides contain primer landing sites for PCR amplification and an address sequence for hybridization to the universal bead array. Because randomers are used in the cDNA synthesis, and the query oligonucleotides target cDNA sequences only 50 nucleotides in length, partially degraded RNAs can be used in the assay. The DASL assay design resembles RT-PCR with highly multiplexed templates, but with only three PCR primers. Because the oligonucleotides all share the same primers, and the amplicons are of a uniform size, the amplification step is expected to maintain an unbiased representation of transcript abundance. This methodology, however, only allows for the interrogation of hundreds of pre-selected genes in a single experiment (the DASL assay can monitor expression of up to 1,536 sequence targets (512 genes at 3 probes per gene) in 50 ng of total RNA derived from FFPET samples; Bibikova et al., supra).

Another protocol for analysis of gene expression profiling in FFPET samples has recently been developed at Arcturus Bioscience, Incorporated (Mountain View, Calif.), and involves isolation and amplification of FFPET RNA using the Paradise™ Reagent System, which is currently sold by Molecular Devices Corporation (Sunnyvale, Calif.). The Paradise® Reagent System has been used to perform gene expression profiling of microdissected human breast cancer cells from FFPET samples (Erlander et al., Abstract No. 498, American Society of Clinical Oncology Annual Meeting, Chicago Ill., May 31, 2003 through Jun. 3, 2003), and gene expression profiling of microdissected colonic epithelial cells from FFPET samples (Coudry et al., J Mol. Diagn. 9:70-79, 2007). The Paradise™ Reagent System is also reported to have an RNA extraction protocol that allows optimized microarray performance when used together with arrays, for example the GeneChip® X3P Array, from Affymetrix, Incorporated (Santa Clara, Calif.) or Agilent Technologies, Incorporated (Santa Clara, Calif.). However, the Paradise™ Reagent System appears to be best suited to relatively fresh paraffin blocks with high molecular weight RNA preserved in the specimen, as opposed to paraffin blocks that are more than a few years old.

Recently a method was described utilizing the TransPlex™ Whole Transcriptome Amplification (“WTA”) kit from Rubicon Genomics, Incorporated (Ann Arbor, Mich.) for whole genome expression analysis of old FFPET samples using the GeneChip® U133-X3P Array (Affymetrix, Incorporated) was described (Paik, supra). The TransPlex™ WTA kit bypasses the need for an intact polyadenylic acid tail by using random primers for cDNA synthesis, and adaptor-based PCR for cDNA amplification. However, this method utilized direct end-labeling of cDNA product from the TransPlex™ WTA kit, and was not reproducible when the number of samples analyzed was expanded.

Therefore, the need remains for a method of analyzing gene expression profiles from FFPET samples that can address multiple genes and samples in one experiment from aged (greater than one year old) FFPET samples.

### BRIEF SUMMARY OF THE INVENTION

The methods of the present invention overcome the shortcomings present in the art by providing protocols that can be used to obtain biological relevant information using high density gene-expression arrays and probes obtained from FFPET nucleic acids. In one embodiment, the present invention provides a method for analyzing gene expression levels from a FFPET sample, comprising pre-hybridizing a labeled nucleic acid sample prepared from the FFPET sample with a first microarray, hybridizing the unbound labeled nucleic acid sample with a second microarray, and detecting the labeled nucleic acid sample bound to the second microarray. In certain aspects of the invention, the first microarray is a previously used microarray, while in other aspects of the present invention, the first microarray is a previously unused or new microarray.

The pre-hybridization can utilize any nucleic acid-based microarray, including, but not limited to, commercially available microarrays, for example microarrays available from Affymetrix, Incorporated, Agilent Technologies, Incorporated, Illumina, Incorporated (San Diego, Calif.), GE Healthcare (Piscataway, N.J.), NimbleGen Systems, Incorporated (Madison, Wis.), Invitrogen Corporation (Carlsbad, Calif.), and the like. While in certain aspects of the present invention the first microarray and the second microarray are from the same manufacturer or source, or even the same type of microarray from the same manufacturer or source, in other aspects the first microarray and the second microarray are from different manufacturers or sources. Thus, in certain embodiments of the present invention, the first microarray is an Affymetrix GeneChip®, for example a human X3P array, human genome U133 Plus 2.0 array, human genome U133A 2.0 array, or a human cancer G110 array, and the second microarray is the same type of Affymetrix GeneChip® or a different type of Affymetrix GeneChip®.

In certain aspects of the present invention, the FFPET sample comes from a human. However, in other embodiments of the present invention, the FFPET sample can come from any source, including, but not limited to, a laboratory animal, a companion animal, or a livestock animal, for example a non-human primate, such as a chimpanzee, gorilla, orangutan, gibbon, monkey, macaque, baboon, mangabey, colobus, langur, marmoset, or lemur, a mouse, rat, rabbit, guinea pig, hamster, cat dog, ferret, fish, cow, pig, sheep, goat, horse, donkey, chicken, goose, duck, turkey, amphibian, or reptile.

While in certain aspects of the present invention, the FFPET sample is an aged FFPET sample, for example, a FFPET sample that is at least one year old, at least two years old, at least three years old, at least four years old, at least five years old, at least six years old, at least seven years old, at least eight years old, at least nine years old, at least ten years old, at least fifteen years old, at least twenty years old, or older, in other aspects of the present invention the FFPET sample is less than one year old, less than 9 months old, less than 6 months old, less than 3 months old, less than two months old, less than one month old, less than two weeks old, less than one week old, or a fresh FFPET sample.

In certain embodiments of the present invention, the labeled nucleic acid sample is prepared from nucleic acids that are isolated from the FFPET sample, for example, RNA or DNA that is isolated from the FFPET sample. In certain aspects of the present invention, the nucleic acids are isolated from the FFPET sample by dissecting, for example macrodissecting or microdissecting, tissue from the FFPET sample in order to create sections, or thin sections, of the FFPET sample. In certain aspects of the present invention the sections are less than 1 micron thick, about 1 micron thick, about 5 microns thick, about 10 microns thick, at least 1 micron thick, at least 5 microns thick, at least 10 microns thick, between about 1 and about 5 microns thick, between about 1 and about 10 microns thick, or between about 5 and about 10 microns thick.

In certain aspects of the present invention, the FFPET sample comprises an area of diseased tissue, for example a tumor or other cancerous tissue, while in other aspects of the present invention, the FFPET sample comprises normal, untreated, placebo-treated, or healthy tissue. In certain embodiments of the present invention, the diseased area or tissue, or an area of the tissue that contains a particular cellular or subcellular feature or structure, is identified in the FFPET sample, or a section thereof, prior to the isolation of nucleic acids, while in other embodiments of the present invention the nucleic acids are isolated from the FFPET sample without identification of the diseased area or tissue, or cellular or subcellular feature or structure. In embodiments of the present invention where a diseased area or tissue, or particular cellular or subcellular feature or structure, is identified in the FFPET sample, or a section thereof, prior to the isolation of nucleic acids, such identification can be by any method known to those of skill in the art to identify a particular disease area, or cellular or subcellular feature or structure, in a tissue sample, or section thereof, including, but not limited to, visual identification, staining, for example hematoxylin and eosin staining, labeling, and the like.

In embodiments of the present invention where nucleic acids are isolated from the FFPET sample, such nucleic acids can be RNA, DNA, or both, and any technique known to those of skill in the art to isolate nucleic acids can be used. In fact, numerous kits for either nucleic acid isolation are commercially available, and are suitable for use in these embodiments of the present invention. However, in certain embodiments of the present invention, kits specifically designed to isolate RNA from a FFPET sample are used.

In certain embodiments of the present invention, the nucleic acids, for example RNA, isolated from the FFPET sample are amplified. In such embodiments, any technique known to those of skill in the art can be used to amplify the nucleic acids. Once again, numerous kits for nucleic acid amplification are commercially available, and are suitable for use in these embodiments of the present invention. Thus, in certain aspects of the present invention, RNA isolated from the FFPET sample is converted into an amplified cDNA sample or an amplified RNA sample.

In certain aspects of the present invention, the amplified nucleic acid sample is labeled. In these aspects of the present invention, any technique known to those of skill in the art can be used to label the amplified nucleic acid sample. Once again, numerous kits for nucleic acid labeling are commercially available, and are suitable for use in these aspects of the present invention. Thus, in certain embodiments of the present invention, the amplified nucleic acid sample is labeled by 5′ or 3′ end labeling, or by direct chemical labeling. Any type of detectable label can be utilized in these aspects of the present invention, including, but not limited to, radioactive, fluorescent, phosphorescent, or visual labels or dyes, enzymatic labels, and chemical or biological labels that are recognized by a specific binding partner or antibody, or fragment thereof, such as biotin.

In certain aspects of the present invention, the labeled amplified cDNA sample is fragmented. In these aspects of the present invention, any technique known to those of skill in the art can be used to fragment the labeled amplified nucleic acid sample. In certain embodiments of the present invention, the labeled amplified nucleic acid sample is purified prior to and/or following fragmentation. In these embodiments of the present invention, any technique known to those of skill in the art can be used to purify the labeled amplified nucleic acid sample and/or the fragmented nucleic acid sample. As is the case above, numerous kits and reagents for nucleic acid purification are commercially available, and are suitable for use in these aspects of the present invention.

Following hybridization of the labeled nucleic acid probe to the second microarray, any bound labeled nucleic acid probe is detected. In certain aspects of the present invention, the second microarray is washed at least a first time following hybridization, using reagents and techniques that are commercially available or otherwise known to those of skill in the art. In certain embodiments of the present invention, the bound labeled nucleic acid is stained or otherwise treated to enable or enhance detection. The method of detection will usually depend upon the type of label used to label the nucleic acid sample, and will be commercially available or otherwise well-known to those of skill in the art.

Thus, certain embodiments of the present invention provide methods for analyzing gene expression levels from a FFPET sample, comprising identifying a disease area within the FFPET sample, dissecting the identified disease area to obtain at least a first section of the diseased area, isolating RNA from the at least a first section of the diseased area, converting the RNA into an amplified cDNA sample, labeling the amplified cDNA sample, purifying the labeled cDNA sample, fragmenting the purified and labeled cDNA sample, purifying the fragmented cDNA sample, pre-hybridizing the fragmented cDNA sample with a first microarray, hybridizing the unbound fragmented cDNA sample with a second microarray, and detecting the fragmented cDNA sample bound to the second microarray.

### BRIEF DESCRIPTION OF THE SEVERAL VIEWS OF THE DRAWINGS

FIG. 1. A flow diagram describing the methodology of one embodiment of the present invention.

FIG. 2A, FIG. 2B, FIG. 2C, FIG. 2D, FIG. 2E, FIG. 2F, FIG. 2G, FIG. 2H, FIG. 2I, FIG. 2J, FIG. 2K, FIG. 2L, FIG. 2M, FIG. 2N, FIG. 2O, FIG. 2P, FIG. 2Q, FIG. 2R, FIG. 2S, FIG. 2T, FIG. 2U, FIG. 2V, FIG. 2W, FIG. 2X, FIG. 2Y, FIG. 2Z, FIG. 2AA, FIG. 2BB, FIG. 2CC, FIG. 2DD, FIG. 2EE, FIG. 2FF, and FIG. 2GG. A listing of SAM positive genes (N=908) identified from 92 FFPET samples analyzed using one embodiment of the present invention using a U133 X3P whole genome expression array from Affymetrix, Incorporated.

FIG. 3A, FIG. 3B, FIG. 3C, FIG. 3D, FIG. 3E, FIG. 3F, FIG. 3G, FIG. 3H, FIG. 3I, FIG. 3J, FIG. 3K, FIG. 3L, FIG. 3M, FIG. 3N, FIG. 3O, FIG. 3P, FIG. 3Q, FIG. 3R, FIG. 3S, FIG. 3T, FIG. 3U, FIG. 3V, FIG. 3W, FIG. 3X, FIG. 3Y, FIG. 3Z, FIG. 3AA, FIG. 3BB, FIG. 3CC, FIG. 3DD, FIG. 3EE, FIG. 3FF, FIG. 3GG, FIG. 3HH, FIG. 3II, FIG. 3JJ, FIG. 3KK, FIG. 3LL, FIG. 3MM, FIG. 3NN, FIG. 3OO, FIG. 3PP, FIG. 3QQ, and FIG. 3RR. A listing of SAM positive genes (N=1319) identified from 92 FFPET samples analyzed using one embodiment of the present invention using a U133 Plus 2.0 whole genome expression array from Affymetrix, Incorporated.

### DETAILED DESCRIPTION OF THE INVENTION

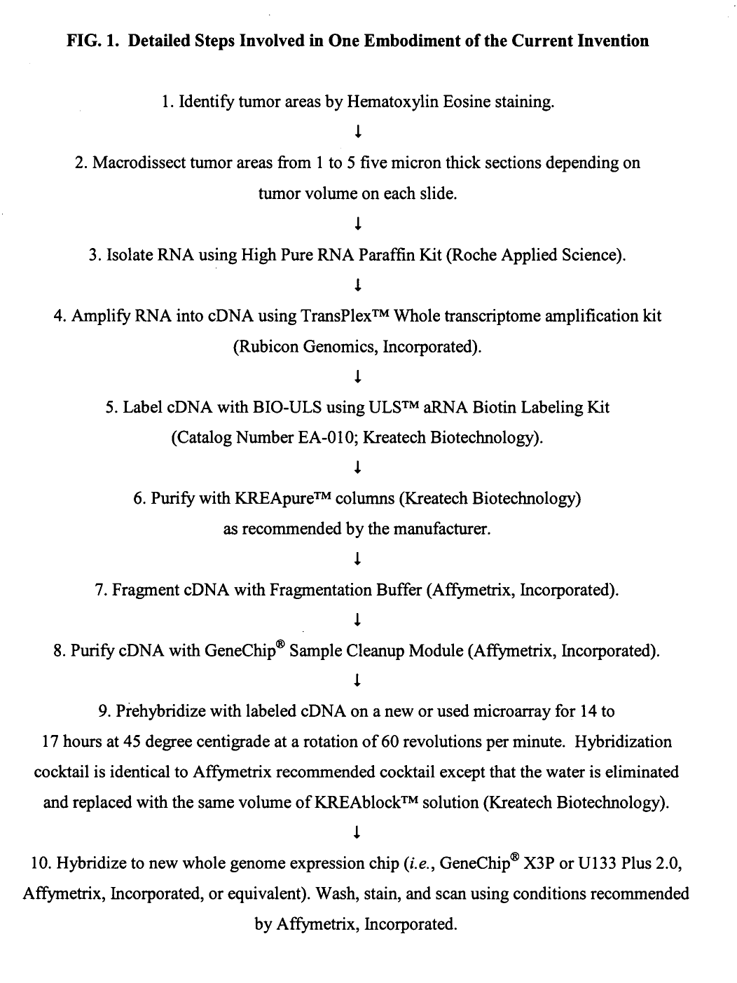

Formalin-fixed, paraffin-embedded tissue (FFPET) samples represent the most commonly collected and stored samples for use in the diagnosis and prognosis of disease, including cancer. Nevertheless, historically these samples have been underutilized for the purpose of gene expression profiling because of the poor quality and quantity of FFPET nucleic acids. The methods of the present invention overcome these and other problems and provide protocols that can be used to obtain biologically relevant, whole genome-expression information using high density gene-expression arrays and labeled probes obtained or created from nucleic acids isolated from new or aged (greater than one year old) FFPET tissues. Using the techniques of the present invention in a microarray based analysis, 60,000 genes can be interrogated in 32 samples in a single experiment, and completed in a 3 day period. FIG. 1 shows a flow chart for one embodiment of the present invention.

The present invention is thus applicable to basic research aimed at the discovery of gene expression profiles relevant to the diagnosis and prognosis of disease. However, the present invention is also applicable to other fields of research where the quality of nucleic acid is poor, such as forensics, archeology, medical history, and paleontology.

The present invention provides methods for analyzing gene expression levels from FFPET samples that comprise pre-hybridizing the labeled nucleic acid sample prepared from a FFPET sample with a first microarray, and then hybridizing the portion of the labeled nucleic acid sample that does not bind to the first microarray with a second microarray prior to detection of the labeled nucleic acid sample that binds to the second microarray. Although the first microarray can be a previously unused or new microarray, such microarrays are expensive. Importantly, from a cost-savings perspective, the inventors determined that when the first microarray used for the pre-hybridization step is a previously used microarray, the results of the subsequent hybridization on a second microarray are nearly identical to the results obtained when the pre-hybridization was carried out using a new or previously unused microarray.

Without being limited to any particular theory of the invention, the fact that nucleic acids isolated from FFPET samples are moderately to heavily degraded, particularly as the FFPET samples age, generally precludes the use of an oligo-dT primer to amplify mRNA isolated from the FFPET sample, or produce and/or amplify cDNA produced from mRNA isolated from the FFPET sample. Therefore, random priming techniques are commonly used to produce a sufficient quantity of nucleic acid products from the FFPET sample for subsequent labeling. However, since random priming will also serve to amplify rRNA present in the isolated nucleic acids, labeled products from the amplified rRNA can mask or otherwise reduce the quality of the signal produced when hybridizing labeled nucleic acids from FFPET samples onto a microarray. Incorporating a “pre-hybridization” (or “first hybridization”) of the labeled nucleic acid sample results in an increase in the specific gene signals in subsequent hybridizations with high density gene expression arrays.

Any nucleic acid-based microarray can be used with the methods of the present invention, for the pre-hybridization and any subsequent hybridizations, including, but not limited to, commercially available microarrays, for example microarrays available from Affymetrix, Incorporated, Agilent Technologies, Incorporated, Illumina, Incorporated (San Diego, Calif.), GE Healthcare (Piscataway, N.J.), NimbleGen Systems, Incorporated (Madison, Wis.), Invitrogen Corporation (Carlsbad, Calif.), and the like. Using the methods of the present invention, the pre-hybridization and any subsequent hybridization can utilize microarrays from the same manufacturer or source, or even the same type of microarray from the same manufacturer or source, or microarrays from different manufacturers or sources. Thus, for example, the pre-hybridization could utilize a new, previously unused, or used Affymetrix GeneChip®, for example a human X3P array, human genome U133 Plus 2.0 array, human genome U133A 2.0 array, or a human cancer G110 array, and subsequent hybridizations could utilize the same type or a different type of Affymetrix GeneChip®, or a completely different type of nucleic acid-based micro array.

FFPET samples from any source can be used with the methods of the present invention, including, but not limited to, FFPET samples from human tissues, laboratory animal tissues, companion animal tissues, or livestock animal tissues. Thus, FFPET samples from, for example, a non-human primate, such as a chimpanzee, gorilla, orangutan, gibbon, monkey, macaque, baboon, mangabey, colobus, langur, marmoset, or lemur, a mouse, rat, rabbit, guinea pig, hamster, cat dog, ferret, fish, cow, pig, sheep, goat, horse, donkey, chicken, goose, duck, turkey, amphibian, or reptile can be used in the methods of the present invention. In addition, FFPET samples of any age can be used with the methods of the present invention, including, but not limited to, FFPET samples that are fresh, less than one week old, less than two weeks old, less than one month old, less than two months old, less than three months old, less than six months old, less than 9 months old, less than one year old, at least one year old, at least two years old, at least three years old, at least four years old, at least five years old, at least six years old, at least seven years old, at least eight years old, at least nine years old, at least ten years old, at least fifteen years old, at least twenty years old, or older.

In the methods of the present invention, the labeled nucleic acid sample can be prepared directly or indirectly from nucleic acids, for example RNA, isolated from FFPET samples, or from sections that are prepared from FFPET samples. The sections can be of any desired thickness, depending on the volume of the desired area. Thus, the sections can be thin sections or thick sections, including, but not limited to, sections that are less than 1 micron thick, about 1 micron thick, about 2 microns thick, about 3 microns thick, about 4 microns thick, about 5 microns thick, about 6 microns thick, about 7 microns thick, about 8 microns thick, about 9 microns thick, or about 10 microns thick, depending upon the desired application. In applications where the exact thickness of the section is less important, the sections can be, for example, at least 1 micron thick, at least 2 microns thick, at least 3 microns thick, at least 4 microns thick, at least 5 microns thick, at least 6 microns thick, at least 7 microns thick, at least 8 microns thick, at least 9 microns thick, or at least 10 microns thick. In applications where a range of thicknesses can be utilized, the sections can be defined by a range of sizes, including, but not limited to, between about 1 and about 5 microns thick, between about 1 and about 10 microns thick, or between about 5 and about 10 microns thick.

In many cases, FFPET samples comprise an area of diseased tissue, for example a tumor or other cancerous tissue. While such FFPET samples find utility in the methods of the present invention, FFPET samples that do not comprise an area of diseased tissue, for example FFPET samples from normal, untreated, placebo-treated, or healthy tissues, also can be used in the methods of the present invention. In certain methods of the present invention, a desired diseased area or tissue, or an area containing a particular region, feature or structure within a particular tissue, is identified in a FFPET sample, or a section or sections thereof, prior to isolation of nucleic acids, in order to increase the percentage of nucleic acids obtained from the desired region. Such regions or areas can be identified using any method known to those of skill in the art, including, but not limited to, visual identification, staining, for example hematoxylin and eosin staining, labeling, and the like. In any event, the desired area of the FFPET sample, or sections thereof, can be dissected, either by macrodissection or microdissection, to obtain the starting material for the isolation of a nucleic acid sample.

Any technique known to those of skill in the art can be used to isolate nucleic acids from the FFPET samples. In fact, numerous reagents and/or kits for nucleic acid isolation are commercially available, and are suitable for use in the methods of the present invention. Examples of such kits include, but are not limited to, the PicoPure™ RNA Isolation Kit from Arcturus Bioscience, Incorporated, the High Pure RNA Paraffin Kit from Roche Diagnostics Corporation, Roche Applied Science, and RNA Isolation Kits from Ambion, Incorporated (Austin, Tex.). Certain commercially available kits are specifically designed to isolate nucleic acids, for example RNA, from FFPET samples, and such kits can also be used in certain of the present methods. One of ordinary skill in the art, will readily recognize that a wide variety of techniques, including other common laboratory techniques or other commercially available kits, are capable of isolating nucleic acids from FFPET samples, and can therefore be used in certain methods of the present invention.

In certain methods of the present invention, the isolated nucleic acids are amplified, creating an amplified nucleic acid sample. For example, RNA isolated from FFPET samples can be amplified directly using commercially available kits or reagents, including, but not limited to, the Paradise™ Reagent System (Arcturus Bioscience, Incorporated) and the SenseAMP or SenseAMP Plus Kits (Genisphere, Incorporated, Hatfield, Pa.), each of which utilize T7 polymerase to amplify RNA, the RampUP or RampUP Plus Kits (Genisphere, Incorporated), which utilize both T7 and T3 promoters to amplify RNA, or any other methods known to those of skill in the art. In other methods of the present invention, RNA isolated from FFPET samples can be converted into cDNA and then amplified, using commercially available reagents or kits, including, but not limited to, the TransPlex™ Whole Transcriptome Amplification Kit (Rubicon Genomics, Incorporated), the WT-Ovation™ FFPE System or WT-Ovation™ Pico RNA Amplification System (NuGEN Technologies, Incorporated, San Carlos, Calif.), the GeneChip® WT cDNA Synthesis and Amplification Kit (Affymetrix, Incorporated), the MessageAmp™ II aRNA Amplification Kits (Ambion, Incorporated), or any other methods known to those of skill in the art. One of ordinary skill in the art will readily recognize that a wide variety of techniques, including other common laboratory techniques or other commercially available kits, are capable of amplifying RNA, cDNA, or DNA without depending on polyadenylated tail of MRNA, and can therefore be used in certain methods of the present invention.

In certain embodiments of the present invention, the amplified nucleic acid sample can be labeled for identification or visualization within a microarray. Any of various common laboratory techniques for labeling DNA, RNA, or both, known to those of skill in the art can be used to label the amplified nucleic acid sample, including, but not limited to, 5′ or 3′ end labeling, direct chemical labeling, synthesis with labeled nucleotides or pseudo-nucleotides, biotin conjugate labeling, as well as random primer or specific primer labeling. Numerous techniques, reagents, and kits for nucleic acid labeling are commercially available, including, but not limited to, labeling the amplified nucleic acid sample with Biotin-ULS ULS using ULS™ aRNA Biotin Labeling Kit (catalog number EA-010; Kreatech Biotechnology, Amsterdam, The Netherlands), the FL-Ovation™ cDNA Biotin Module V2 (NuGEN Technologies, Incorporated), or the GeneChips® WT Terminal Labeling Kit (Affymetrix, Incorporated), and are suitable for use in certain methods of the present invention. Any type of detectable label can be utilized in these aspects of the present invention, including, but not limited to, radioactive, fluorescent, phosphorescent, or visual labels or dyes, enzymatic labels, and chemical or biological labels that are recognized by a specific binding partner or antibody, or fragment thereof. If desired, the labeled, amplified nucleic acid sample can be purified, using any of the numerous nucleic acid purification techniques known to those of skill in the art. As is the case above, numerous techniques, kits and reagents for nucleic acid purification are commercially available, including, but not limited to, KREApure™ columns (Kreatech Biotechnology), and GeneChip® Sample Cleanup Module (Affymetrix, Incorporated), and are generally used as recommended by the manufacturer.

If desired, in certain methods of the present invention the labeled nucleic acid sample can be fragmented prior to labeling. Such fragmentation can use any of a number of techniques known to those of skill in the art, including, but not limited to, chemical treatment, for example using an alkaline solution, enzyme treatment, or mechanical treatment, such as shearing or sonication, or use commercially available techniques, reagents, and/or kits, as exemplified by the GeneChip® WT Terminal Labeling Kit (Affymetrix, Incorporated). As described above, in certain methods of the present invention the fragmented and labeled nucleic acid sample can be purified, using any of the numerous nucleic acid purification techniques known to those of skill in the art. As detailed above, numerous techniques, kits and reagents for nucleic acid purification are commercially available, including, but not limited to, KREApure™ columns (Kreatech Biotechnology), and GeneChip® Sample Cleanup Module (Affymetrix, Incorporated).

The labeled nucleic acid sample can then be used in a pre-hybridization with a new, previously unused, or used microarray. One of ordinary skill in the art will readily recognize that the pre-hybridization step may be performed using a variety of different buffers and under a variety of temperatures, times, and other conditions, which will generally depend on the particular microarray used in the pre-hybridization step. Optimization of such conditions is standard procedure in molecular biology laboratories. In certain embodiments, the microarray is pre-hybridized for 14 to 17 hours at 45° C. at a rotation of 60 revolutions per minute. In certain embodiments, the pre-hybridization cocktail is the same as the hybridization cocktail recommended by Affymetrix, Incorporated (GeneChip® Hybridization, Wash, and Stain Kit), with the exception that the water is eliminated and replaced with an equal volume of KREAblock™ solution (Kreatech Biotechnology).

After pre-hybridization the pre-hybridization cocktail containing unbound labeled nucleic acid sample (the portion of the labeled nucleic acid sample that does not bind to the pre-hybridization microarray) is used to hybridize to a different microarray. One of ordinary skill in the art will readily recognize that the hybridization step may be performed using a variety of different buffers and under a variety of temperatures, times, and other conditions, which will generally depend on the particular microarray used in the hybridization step. Optimization of such conditions is standard procedure in molecular biology laboratories. In certain embodiments, the microarray is hybridized for 14 to 17 hours at 45° C. at a rotation of 60 revolutions per minute.

After hybridization, the microarray chip is analyzed for positive probe binding, in certain cases after washing the microarray at least once. One of ordinary skill in the art will readily recognize that washing and detection of probe binding may be performed using a variety of different buffers and under a variety of temperatures, times, and other conditions, which will generally depend on the particular nucleic acid label and microarray used. Optimization of such conditions is standard procedure in molecular biology laboratories. In methods that utilize a commercially available microarray for the hybridization step, the particular washing (if required) and detection conditions are provided by the manufacturer of the particular microarray. In certain methods of the present invention that utilize a GeneChip® (Affymetrix, Incorporated) microarray for the hybridization step, the microarray can be washed, stained, and/or scanned using the GeneChip® Hybridization, Wash, and Stain Kit (Affymetrix, Incorporated).

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Such modifications are intended to fall within the scope of the appended claims.

### EXAMPLE 1

The effect of a pre-hybridization step on detection of specific gene signals in a subsequent hybridization on a high density gene expression array was tested by comparing the percent present call (proportion of probes on a microarray that yield meaningful above background data within each experiment) for 25 FFPET samples from breast cancer patients using a GeneChip® U133-X3P Array (Affymetrix, Incorporated) with and without pre-hybridization to a GeneChip® U133 Plus 2.0 Array (Affymetrix, Incorporated).

The methodology used in this study is outlined in FIG. 1. Briefly, the tumor area was identified in each of the 25 FFPET samples by hematoxylin-eosin staining, and the tumor areas were macrodissected from thin sections (1 to 5 microns thick, depending on the tumor volume) generated from each of the FFPET samples. RNA was isolated from the macrodissected tumor areas using the High Pure RNA Paraffin Kit (Roche Diagnostics Corporation, Roche Applied Science), following the instructions provided by the manufacturer. The RNA isolated from each of the FFPET samples was then amplified using the TransPlex™ Whole Transcriptome Amplification Kit (Rubicon Genomics, Incorporated), following the instructions provided by the manufacturer, and the resulting cDNA samples were labeled with BIO-ULS using the ULS™ aRNA Biotin Labeling Kit (catalog number EA-010; Kreatech Biotechnology), following the instructions provided by the manufacturer. The labeled cDNA samples were purified using KREApure™ columns (Kreatech Biotechnology), as recommended by the manufacturer, and then fragmented and purified using the GeneChip® Sample Cleanup Module (Affymetrix, Incorporated), and then pre-hybridized on a new (fresh) GeneChip® U133 Plus 2.0 Arrays (Affymetrix, Incorporated) for 14 to 17 hours at 45° C. at a rotation of 60 revolutions per minute. The pre-hybridization cocktail was identical to the hybridization cocktail recommended by Affymetrix, Incorporated, except that water was eliminated and replaced with the same volume of KREAblock™ solution (Kreatech Biotechnology). The pre-hybridization cocktail (containing the unbound portion of the labeled cDNA sample) from each sample was then used to hybridize to a new (fresh) GeneChip® U133-X3P Array (Affymetrix, Incorporated). The arrays were washed and stained using the GeneChip® Hybridization, Wash, and Stain Kit (Affymetrix, Incorporated), and then scanned using the conditions recommended by Affymetrix, results are shown in Table 1.

TABLE 1

| FFPET | Percent Present Call | Percent Present Call |
|---|---|---|
| Sample | (Without Pre-hybridization) | (With Pre-hybridization) |
| 39 | 11.8 | 16.4 |
| 45 | 11.7 | 18.7 |
| 67 | 14.1 | 18.3 |
| 92 | 12.9 | 18.5 |
| 133 | 14.7 | 13.5 |
| 148 | 10.8 | 12.4 |
| 153 | 10.3 | 9.7 |
| 164 | 17.7 | 17.3 |
| 1274 | 10.6 | 17.9 |
| 1408 | 12.3 | 24.2 |
| 1334 | 15.3 | 22.2 |
| 1335 | 10.8 | 22.3 |
| 1477 | 11.5 | 23.3 |
| 1486 | 14.8 | 24.0 |
| 1498 | 13.8 | 23.4 |
| 1502 | 12.1 | 22.0 |
| 1531 | 11.8 | 22.0 |
| 1564 | 10.5 | 18.9 |
| 1639 | 7.2 | 9.5 |
| 1652 | 11.5 | 17.9 |
| 1685 | 11.4 | 19.0 |
| 1704 | 10.2 | 15.2 |
| 1729 | 11.3 | 22.5 |
| 1778 | 12.2 | 20.2 |
| 1791 | 10.3 | 22.8 |
| Average Percent | 12.064 | 18.884 |
| Present Call | | |

The results show a statistically significant (p=3.9E-9 by t-test) improvement of percent present call when a pre-hybridization step was included. When a subsequent hybridization was performed using the very same labeled cDNA sample/hybridization cocktail on a new (fresh) GeneChip® U133 Plus 2.0 Array (Affymetrix, Incorporated), the percent present call was higher than the percent present call without pre-hybridization, which also utilized a GeneChip® U133 Plus 2.0 Array (Affymetrix, Incorporated). The results are shown in Table 2.

TABLE 2

| | Percent Present Call | Percent Present | Percent Present Call |
|---|---|---|---|
| FFPET | U133 Plus 2.0 Without | Call X3P With | U133 Plus 2.0 With |
| Sample | Pre-hybridization | Pre-hybridization | Pre-hybridization |
| 1334 | 15.3 | 26.8 | 22.2 |
| 1335 | 10.8 | 21.8 | 22.3 |
| 1408 | 12.3 | 27.1 | 24.2 |
| 1477 | 11.5 | 25.1 | 23.3 |
| 1486 | 14.8 | 23.4 | 24.0 |
| 1498 | 13.8 | 25.5 | 23.4 |
| 1502 | 12.1 | 26.1 | 22.0 |
| 1531 | 11.8 | 21.8 | 22.0 |
| 1564 | 10.5 | 13.1 | 18.9 |
| 1639 | 7.2 | 9.1 | 9.5 |
| 1652 | 11.5 | 15.5 | 17.9 |
| 1685 | 11.4 | 16.5 | 19.0 |
| 1704 | 10.2 | 13.0 | 15.2 |
| 1729 | 11.3 | 16.6 | 22.5 |
| 1778 | 12.2 | 16.5 | 20.2 |

Removal of non-specific signals in the first hybridization (pre-hybridization) allows for greater detection of differentially expressed genes because during the pre-hybridization the elements responsible for many non-specific signals stuck to the chip and were removed from the sample so that subsequent hybridizations gave a better, more specific signal.

While pre-hybridization on chips covering whole genomes or arrays having substantially overlapping probes increases signal specificity, the use of a whole genome, or gene expression chip, for the purpose of removing non-specific hybridization signals is expensive. Therefore, a previously used gene expression chip was tested to determine if pre-hybridization on a previously used chip removed non-specific hybridization signals. In this experiment, a labeled sample was pre-hybridized on a new/unused chip or on a used chip. The sample that was pre-hybridized to the new chip had a percent present call (17.9) that was nearly identical to the sample that was pre-hybridized to the used chip (17.4). The use of a used gene expression chip for the purpose of filtering a sample represents a novel and cost effective feature of the present invention.

### EXAMPLE 2

In order to compare the methodology described in Example 1, above, with the Paradise™ Reagent System on RNA extractable from paraffin blocks that are more than one year old, the methodology described in Example 1, above, and the Paradise™ Reagent System were tested on fourteen FFPET tumor samples that were between 1 and 10 years old. Significance Analysis of Microarray (SAM), a widely used method to identify genes that are differentially expressed between two phenotypes (Tusher et al., Proc. Natl. Acad. Sci. USA 98:5116-5121, 2001), was used to see if the estrogen receptor (ER) gene ESR1, which is known to be differentially expressed between ER positive and ER negative tumors, could be identified as being a differentially expressed gene between ER positive and ER negative tumors in a dataset generated using the Paradise™ Reagent System, following the instructions provided by the manufacturer, and a dataset generated using the methodology described in Example 1, above. Of the fourteen FFPET tumor samples, three were known to be ER negative, ten were known to be ER positive, and one sample was undetermined as to the ER expression.

The results showed that a dataset generated using the Paradise™ Reagent System failed to identify any differentially expressed genes between the ER positive and ER negative samples, whereas the dataset generated using one of the methods of the present invention identified seven genes that were differentially expressed between the ER positive and ER negative samples (Table 3).

TABLE 3

| Affymetrix Probe | | Gene |
|---|---|---|
| Set ID | Gene Name | Symbol |
| g13111765_3p_a_at | GATA binding protein 3 | GATA3 |
| g4503602_3p_at | estrogen receptor 1 | ESR1 |
| g6652811_3p_at | anterior gradient 2 homolog | AGR2 |
| | (Xenopus laevis) | |
| g7710118_3p_at | engrailed homolog 1 | EN1 |
| Hs.222399.0.S1_3p_a_at | signal peptide, CUB domain, | SCUBE2 |
| | EGF-like 2 | |
| Hs.6612.0.A1_3p_at | cDNA FLJ34896 fis, clone | — |
| | NT2NE2018180 | |
| Hs.79414.1.A1_3p_a_at | SAM pointed domain containing | SPDEF |
| | ets transcription factor | |

In addition, the Paradise™ Reagent System was compared to an embodiment of the present invention with respect to the expression levels of key probe sets that describe estrogen receptor expression and HER2 expression in breast cancer (keratin 7 (KRT7), chemokine (C-X-C motif) ligand 1 (CXCL-1; GRO alpha), keratin 5 (KRT5), estrogen receptor 1 (ESR1), v-erb-b2 erythroblastic leukemia viral oncogene homolog 2 (ERBB2), GATA binding protein 3 (GATA3), and signal peptide, CUB domain, EGF-like 2 (SCUBE2)) using an Affymetrix U133 X3P GeneChip. In unsupervised clustering of the fourteen FFPET samples described above, the gene expression data generated using the Paradise™ Reagent System failed to identify and cluster ER positive versus ER negative tumors, and failed to identify HER2 (ERBB2) positive tumors. In contrast, the embodiment of the current invention correctly identified and clustered the ER positive versus ER negative tumors, and identified a HER2 positive tumor, which also happened to be ER positive. These observations clearly demonstrate that the Paradise™ Reagent System method failed to produce biologically meaningful data from old FFPET samples, whereas the embodiment of the present invention produced biologically meaningful results.

### EXAMPLE 3

To prove that methods of the present invention also work with oligonucleotide arrays from Agilent Technologies, Incorporated, 16 FFPET samples from breast cancer patients were prepared as described in Example 1, above, and analyzed using oligonucleotide arrays from Agilent Technologies, Incorporated.

Using Significance Analysis of Microarray (SAM), as described in Example 2, above, 35 genes were found to be differentially expressed between estrogen receptor positive versus negative cases with false discovery rate of zero. The genes included multiple probes for estrogen receptor gene (ESR1) as well as other known ER related genes such as GATA3, AREG, MAPT, and GSTM3. The results are shown in Table 4.

TABLE 4

| | | Numerator | Denominator | | |
|---|---|---|---|---|---|
| Gene Symbol | Score (d) | (r) | (s + s0) | Fold Change | q-value (%) |
| ESR1 | 6.474708261 | 5.0412 | 0.77859686 | 25.94550481 | 0 |
| ESR1 | 6.466340385 | 5.0471 | 0.78051296 | 25.46381388 | 0 |
| ESR1 | 6.340658263 | 4.8872 | 0.77076974 | 24.27880306 | 0 |
| ESR1 | 6.152270056 | 4.7134 | 0.76612981 | 23.79258312 | 0 |
| TFF1 | 6.150507131 | 5.9033 | 0.95980907 | 94.45828816 | 0 |
| ESR1 | 6.114234598 | 5.0486 | 0.82570638 | 26.16085309 | 0 |
| ESR1 | 6.031393699 | 4.8113 | 0.79771156 | 22.46459598 | 0 |
| ESR1 | 6.003594396 | 4.7073 | 0.78408237 | 22.02622038 | 0 |
| GFRA1 | 5.921552674 | 4.3488 | 0.73440409 | 17.99105209 | 0 |
| ESR1 | 5.91916648 | 4.8344 | 0.81674295 | 22.69872242 | 0 |
| ESR1 | 5.905007266 | 4.7602 | 0.80612729 | 23.58859645 | 0 |
| ESR1 | 5.781180635 | 4.7034 | 0.81357733 | 23.39434246 | 0 |
| TFF1 | 5.419687913 | 5.9756 | 1.10256579 | 122.2599616 | 0 |
| BCMP11 | 4.868800733 | 3.9469 | 0.81065908 | 14.8454219 | 0 |
| KCNK15 | 4.629322848 | 4.1859 | 0.90422242 | 32.20764723 | 0 |
| GATA3 | 4.400783433 | 3.1577 | 0.71752849 | 6.993824938 | 0 |
| ENST00000343518 | 4.168204199 | 4.6249 | 1.10957556 | 19.23073044 | 0 |
| ANKRD21 | 3.835991093 | 4.4838 | 1.1688798 | 7.702632977 | 0 |
| POTE15 | 3.796638871 | 2.9311 | 0.77201509 | 6.911531644 | 0 |
| TFF3 | 3.723154784 | 4.6747 | 1.25557162 | 7.403112268 | 0 |
| TFF3 | 3.709948923 | 4.8281 | 1.30138247 | 6.545833083 | 0 |
| ABAT | 3.707725261 | 2.6643 | 0.71858412 | 7.266140391 | 0 |
| AREG | 3.640540732 | 3.1722 | 0.87135064 | 11.49912023 | 0 |
| ESR1 | 3.636327329 | 2.3751 | 0.65314871 | 3.939882666 | 0 |
| A_24_P913411 | 3.618947446 | 2.7083 | 0.74837022 | 6.567953828 | 0 |
| KCNK15 | 3.612711045 | 3.3326 | 0.92245476 | 18.03654165 | 0 |
| NAT1 | 3.549593753 | 3.4044 | 0.95910623 | 13.26731014 | 0 |
| RTN2 | 3.446799287 | 1.9178 | 0.55640388 | 3.717832787 | 0 |
| CPLX1 | 3.393077065 | 3.2022 | 0.94374146 | 7.801431481 | 0 |
| GREB1 | 3.201420364 | 2.9037 | 0.90699976 | 5.473626723 | 0 |
| FLJ33534 | 3.17972064 | 2.6271 | 0.82619286 | 10.51269465 | 0 |
| SERPINA5 | 3.163992765 | 3.2876 | 1.039055 | 5.331390066 | 0 |
| GSTM3 | 3.145875373 | 2.5384 | 0.80690975 | 6.365091211 | 0 |
| MAPT | 3.14076739 | 2.2368 | 0.71218662 | 4.972095366 | 0 |
| GALNTL1 | 3.128208085 | 2.1403 | 0.68419761 | 4.765051967 | 0 |

When Prediction Analysis of Microarray (PAM) was applied to build a predictor for estrogen receptor status of these 16 cases, 16 probes were selected for the predictor (TFF1, ESR1, ESR1, ESR1, ESR1, ESR1, TFF1, ESR1, ESR1, ESR1, ESR1, GFRA1, ESR1, BCMP11, KCNK15, and ENST00000343518), and on leave one out cross validation, prediction accuracy of 100% was achieved. The performance of PAM predictor in predicting ER status of samples (100% accuracy obtained) is shown in Table 5.

TABLE 5
CV Confusion Matrix (Threshold = 3.43055)

| True/Predicted | 1 | 2 | Class Error Rate |
|---|---|---|---|
| 1 | 8 | 0 | 0 |
| 2 | 0 | 8 | 0 |

Thus, methods of the present invention work on oligonucleotide arrays from Agilent Technologies, Incorporated, as well as those from Affymetrix, Incorporated.

### EXAMPLE 4

In order to compare different techniques for labeling of cDNA samples, cDNA samples were produced from 8 FFPET samples from breast cancer patients, as described in Example 1, above, and labeled either with BIO-ULS using the ULS™ aRNA Biotin Labeling Kit (Kreatech Biotechnology), or end labeled using terminal deoxynucleotide transferase to incorporate biotin-tagged dUTP to the end of the cDNA samples. The labeled samples were then processed as described in Example 1, above, pre-hybridized to a new (fresh) GeneChip® U133 Plus 2.0 Arrays (Affymetrix, Incorporated), and hybridized to a new (fresh) GeneChips® U133-X3P Array (Affymetrix, Incorporated). The arrays were processed as described in Example 1, above, and the results are shown in Table 6.

TABLE 6

| | Percent Present Call | Percent Present Call |
|---|---|---|
| FFPET Sample | BIO-ULS Labeling | End-labeling |
| 39 | 18.4 | 14.4 |
| 45 | 21.8 | 18.4 |
| 67 | 22.2 | 20.8 |
| 92 | 23.7 | 20.4 |
| 1198 | 23.3 | 17.0 |
| 1317 | 22.4 | 19.3 |
| 1351 | 27.4 | 16.5 |
| 1405 | 27.8 | 20.7 |

The results show a statistically significant (p=0.0027 by t-test) improvement of percent present call by labeling with BIO-ULS using the ULS™ aRNA Biotin Labeling Kit (Kreatech Biotechnology) compared to end-labeling using terminal deoxynucleotide transferase to incorporate biotin-tagged dUTP to the end of the cDNA samples.

### EXAMPLE 5

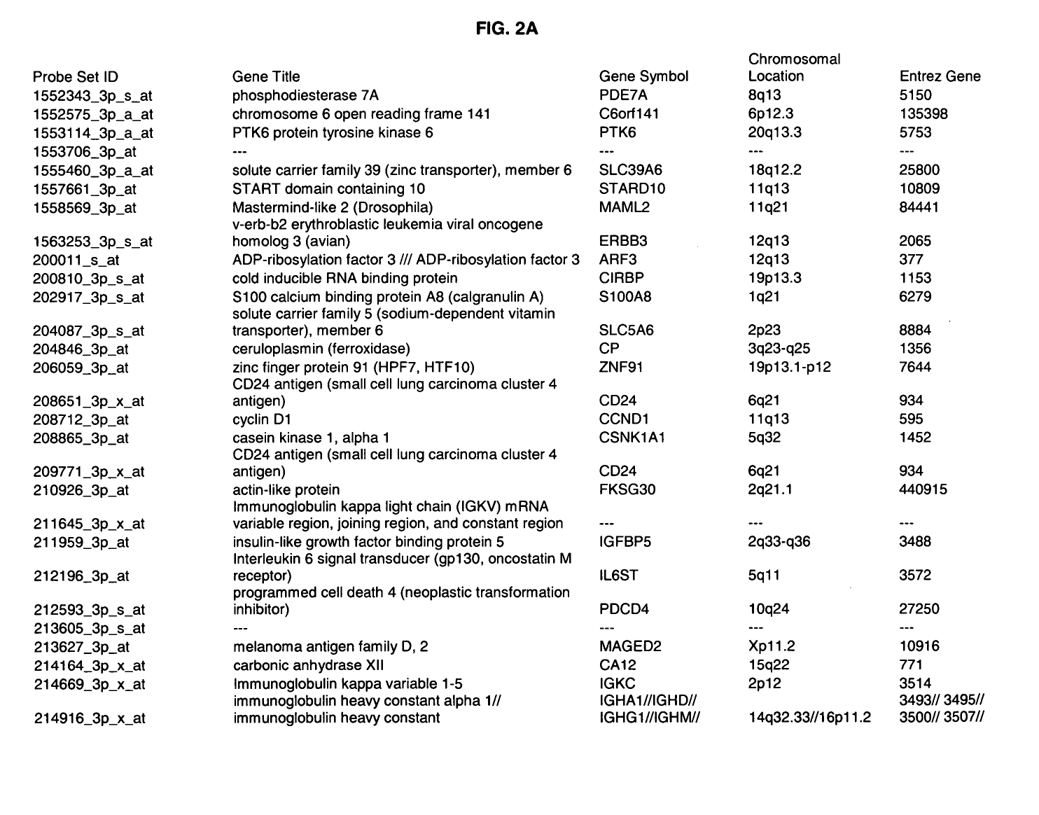

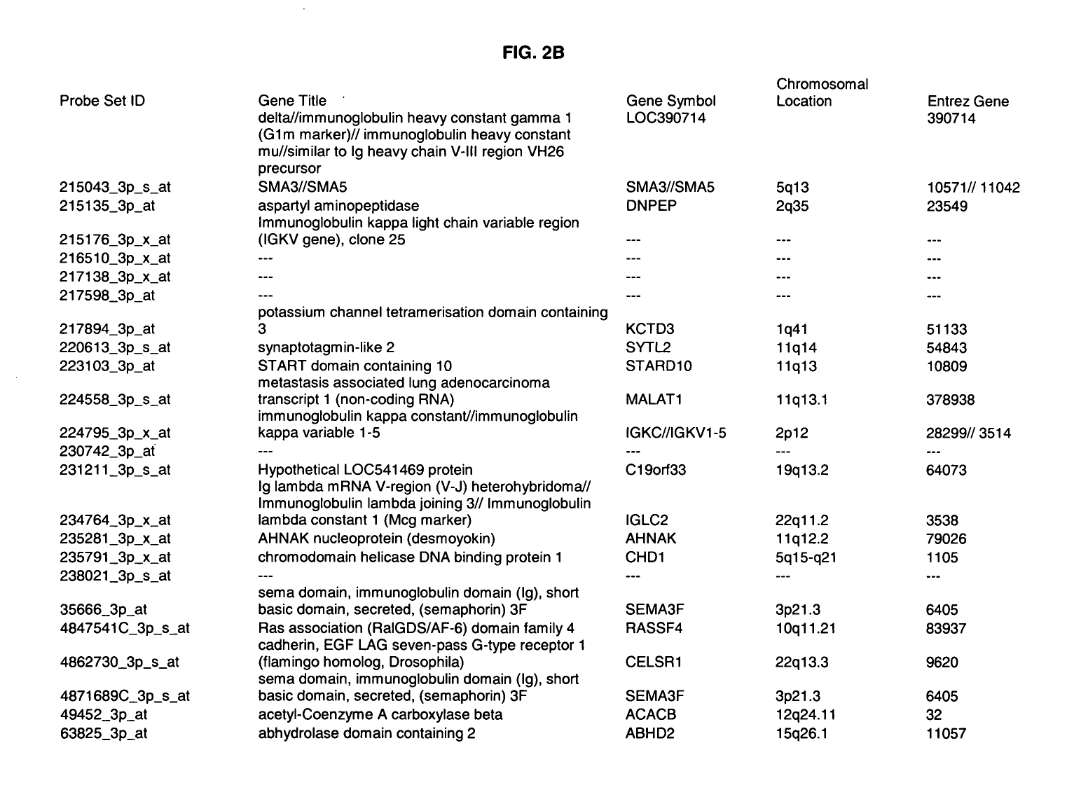

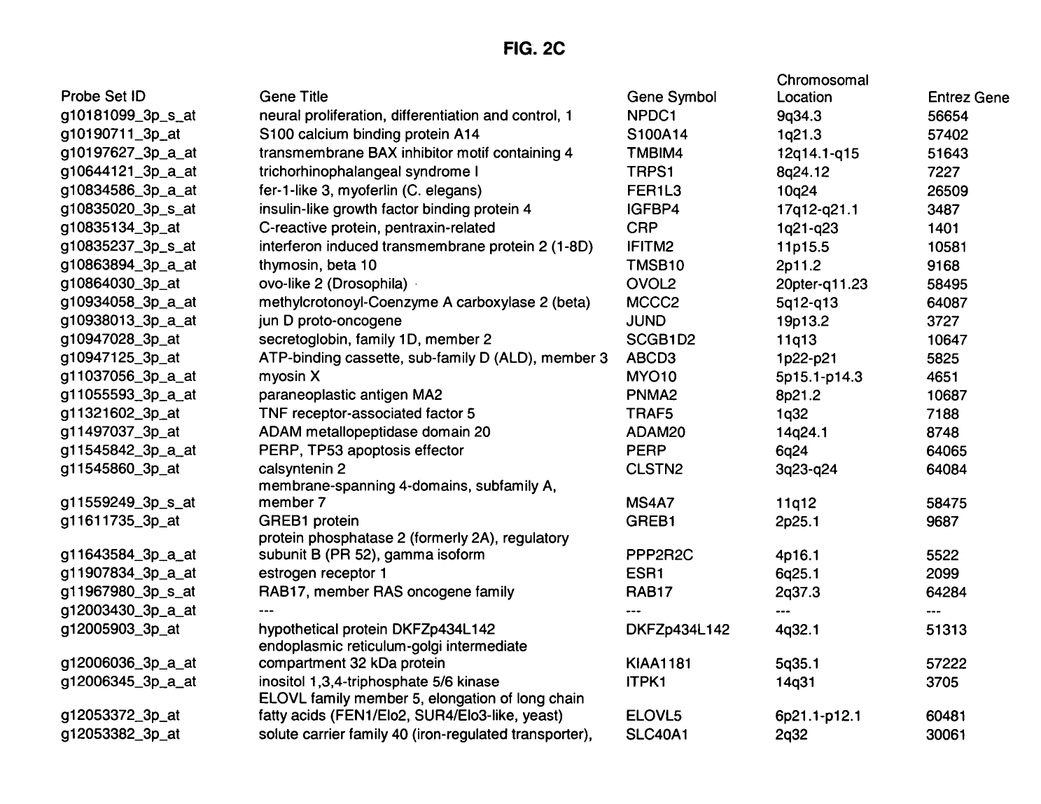

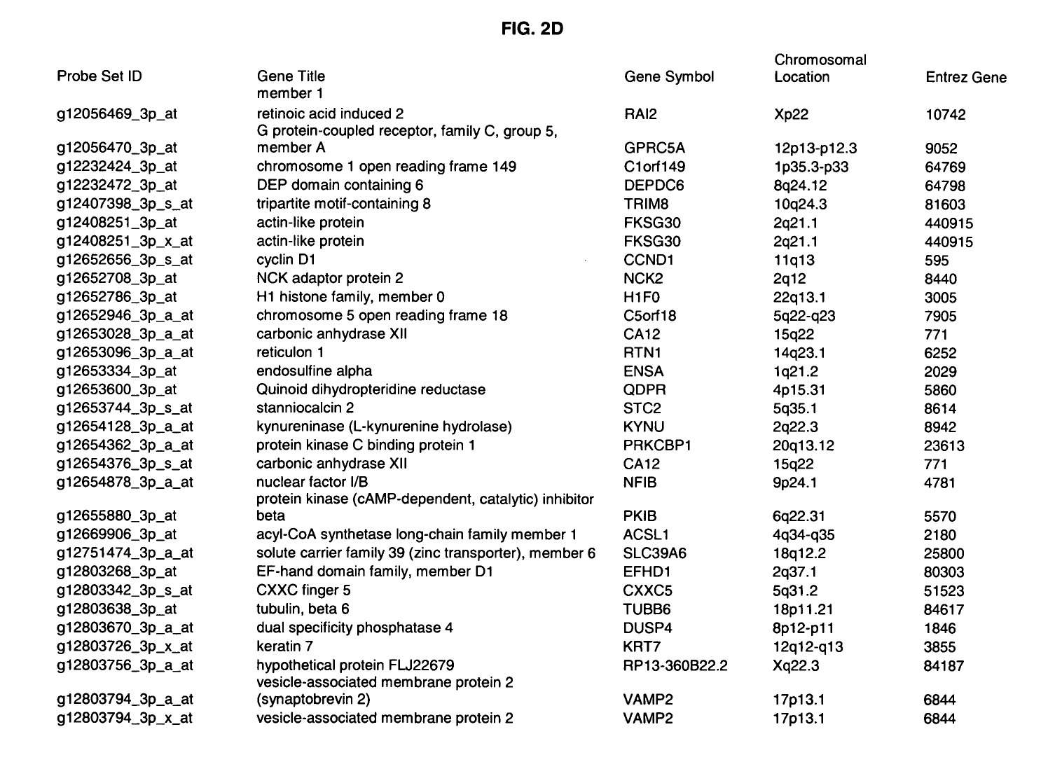

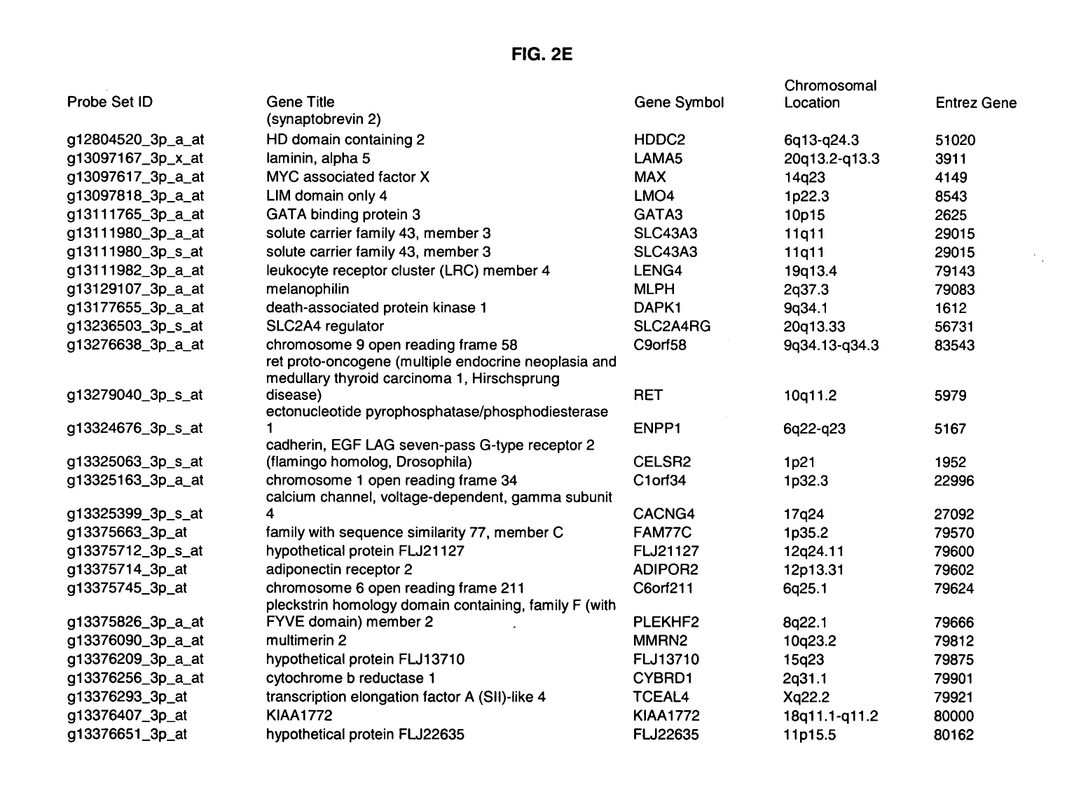

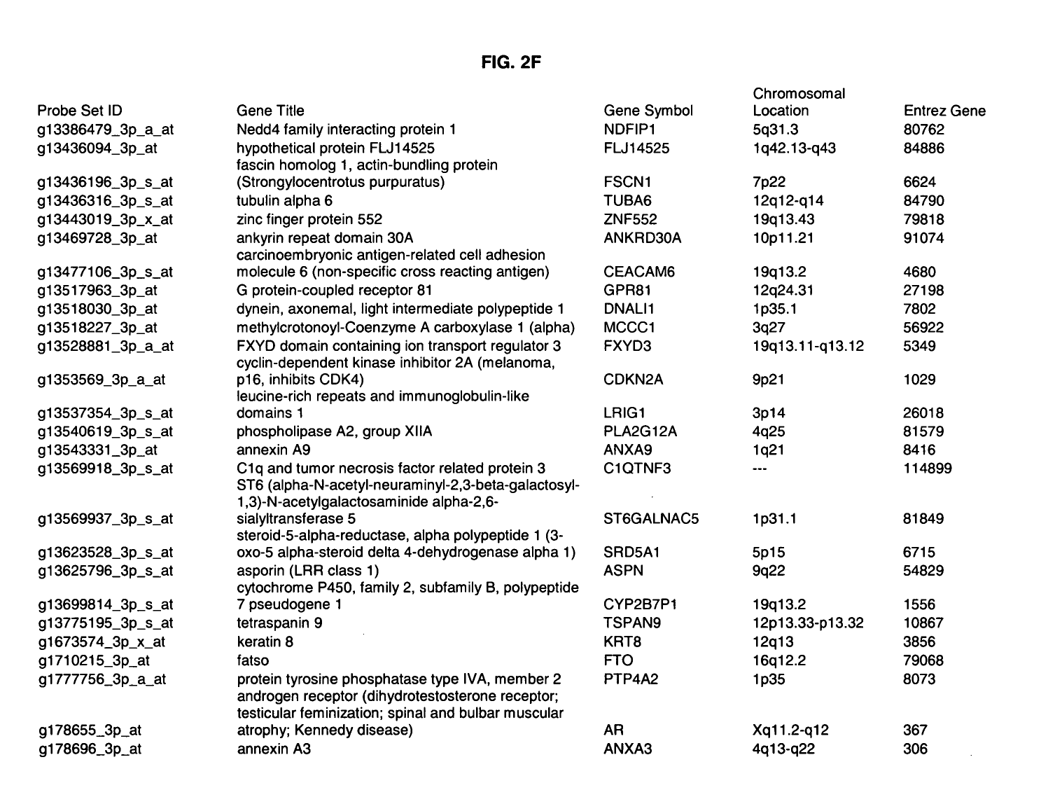

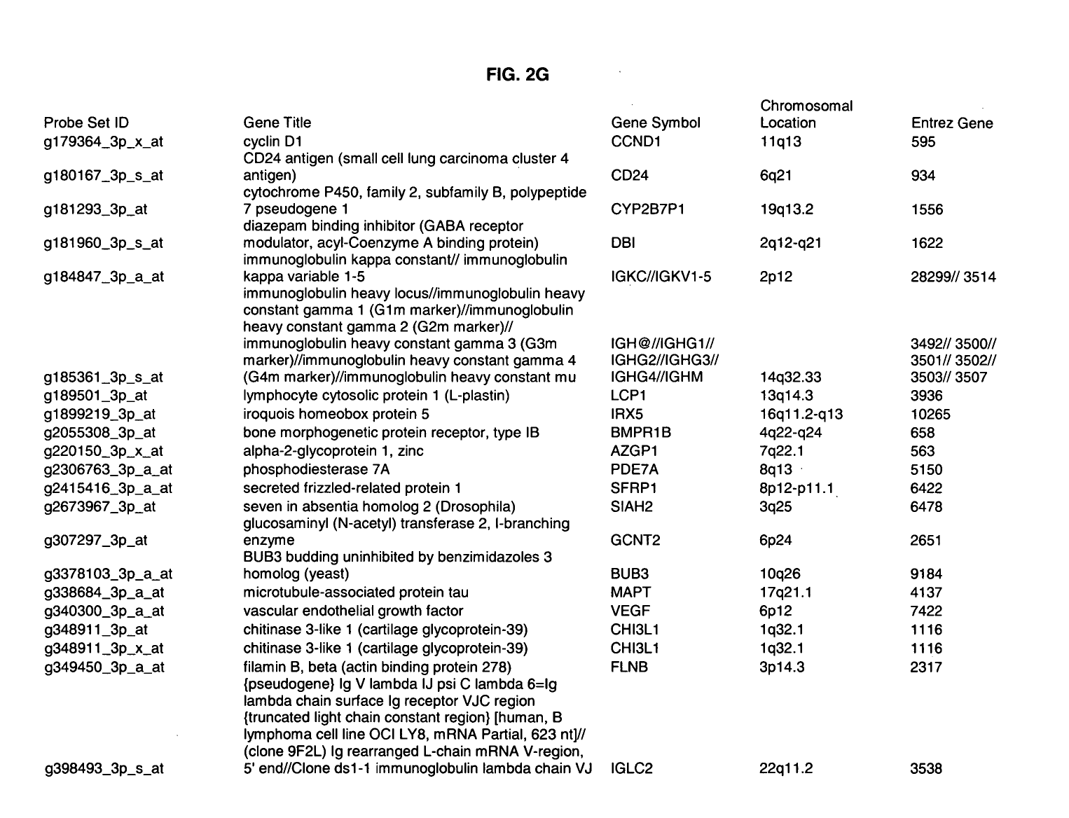

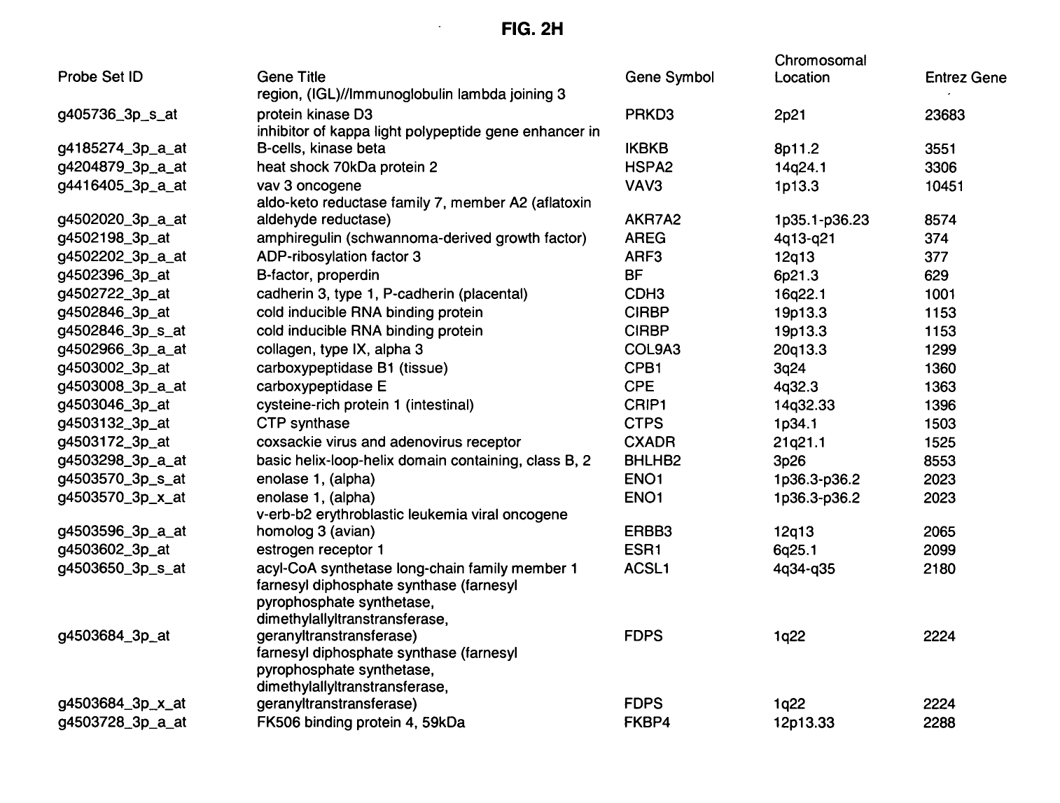

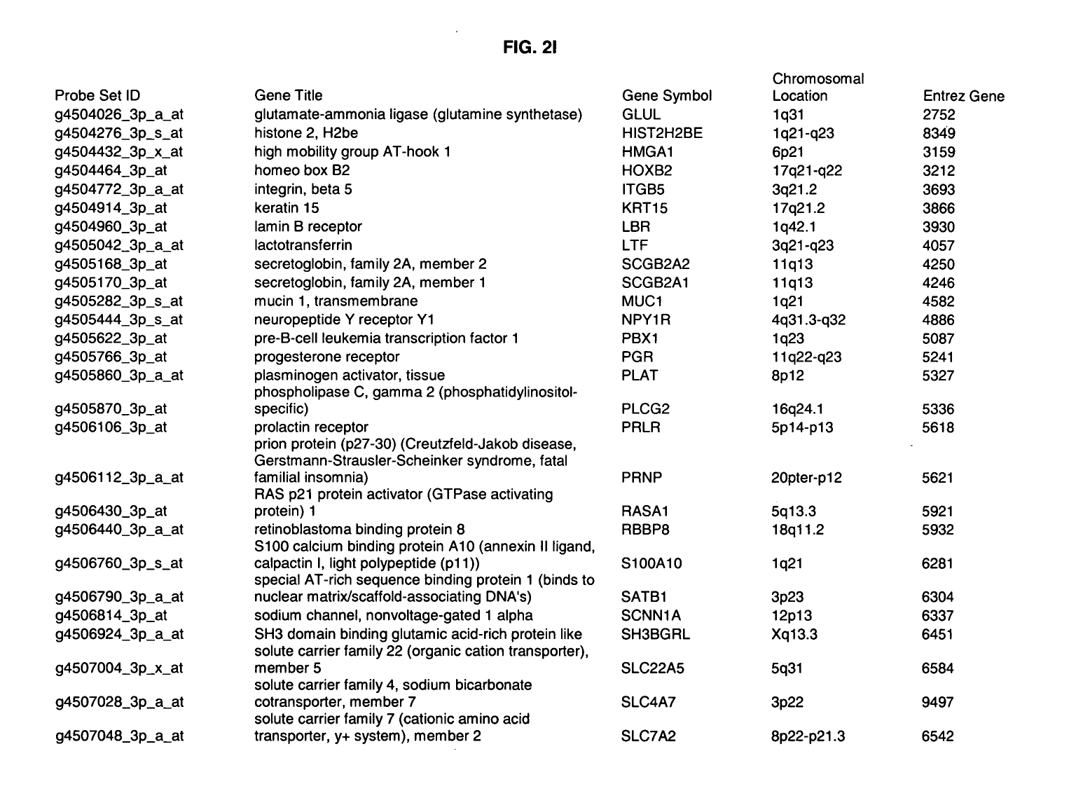

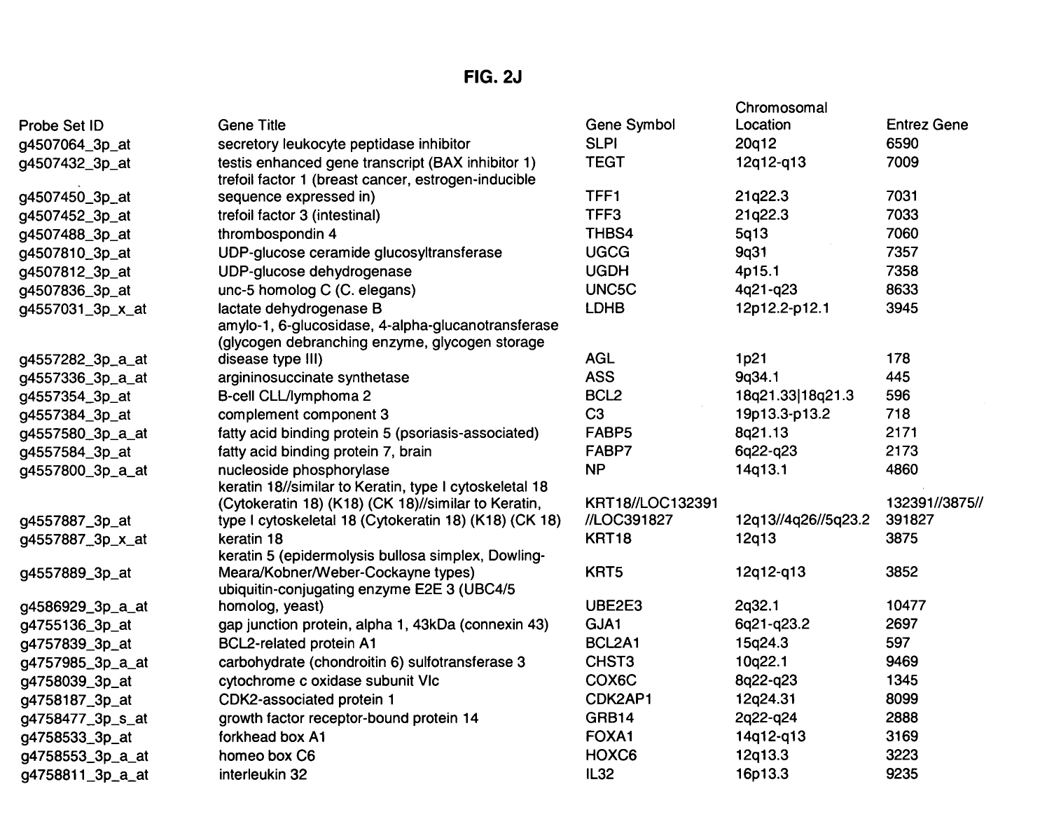

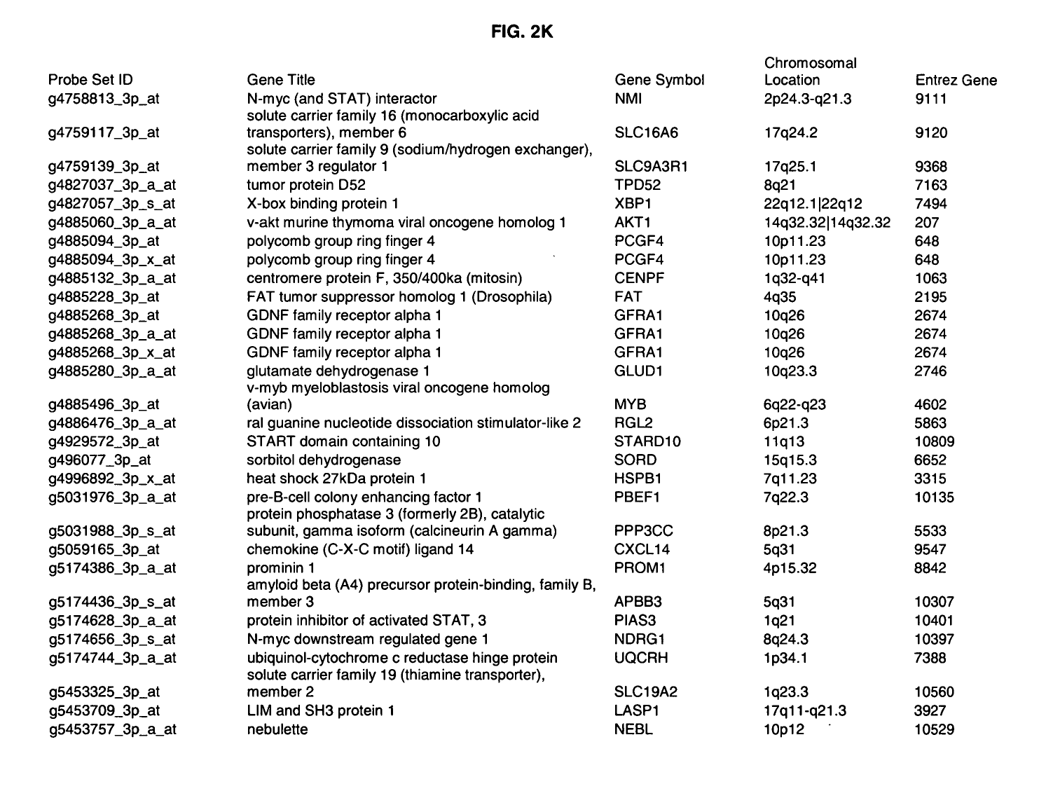

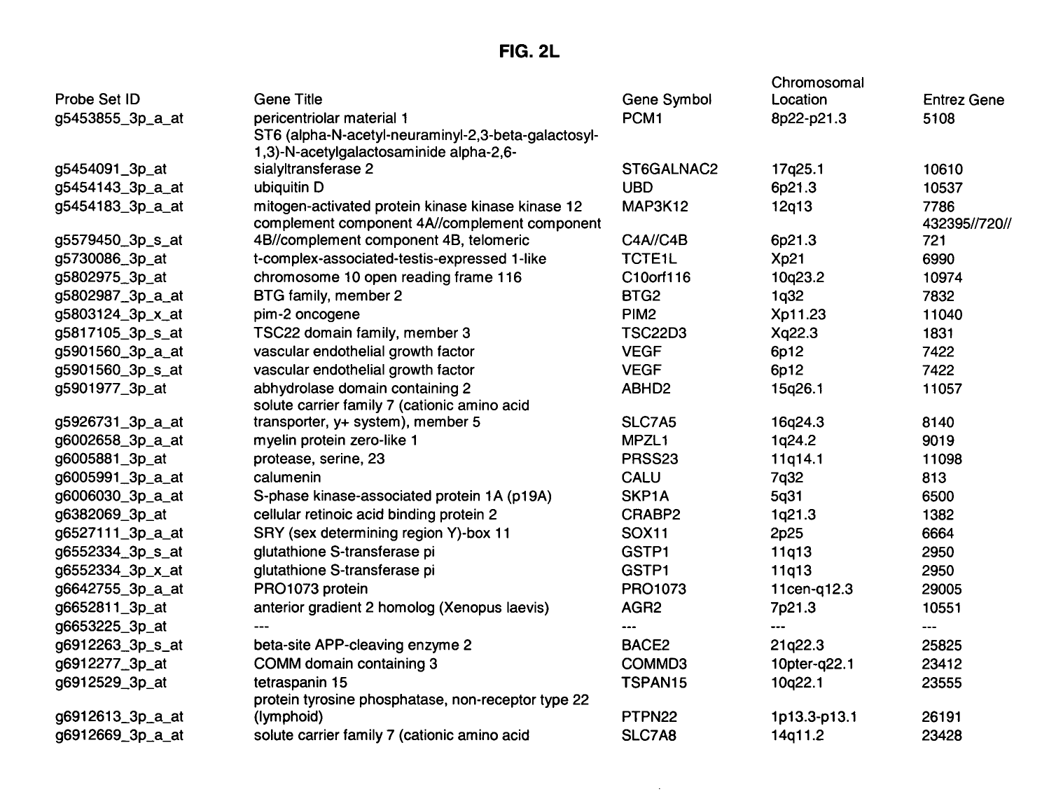

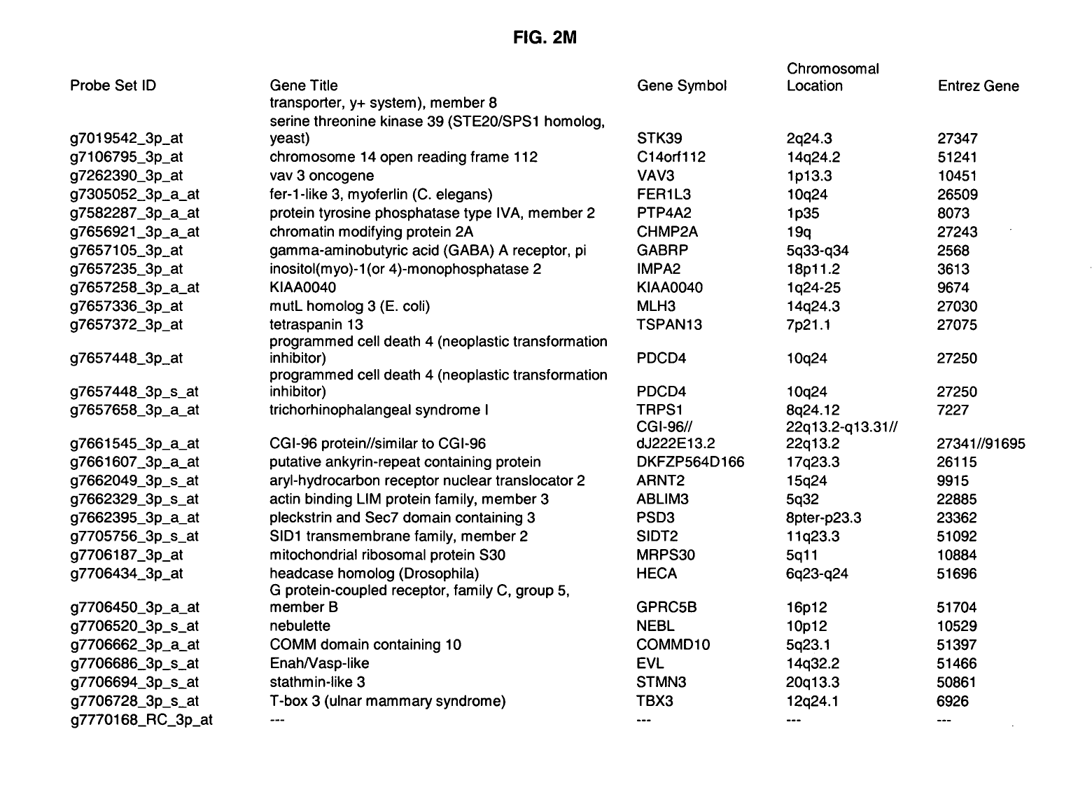

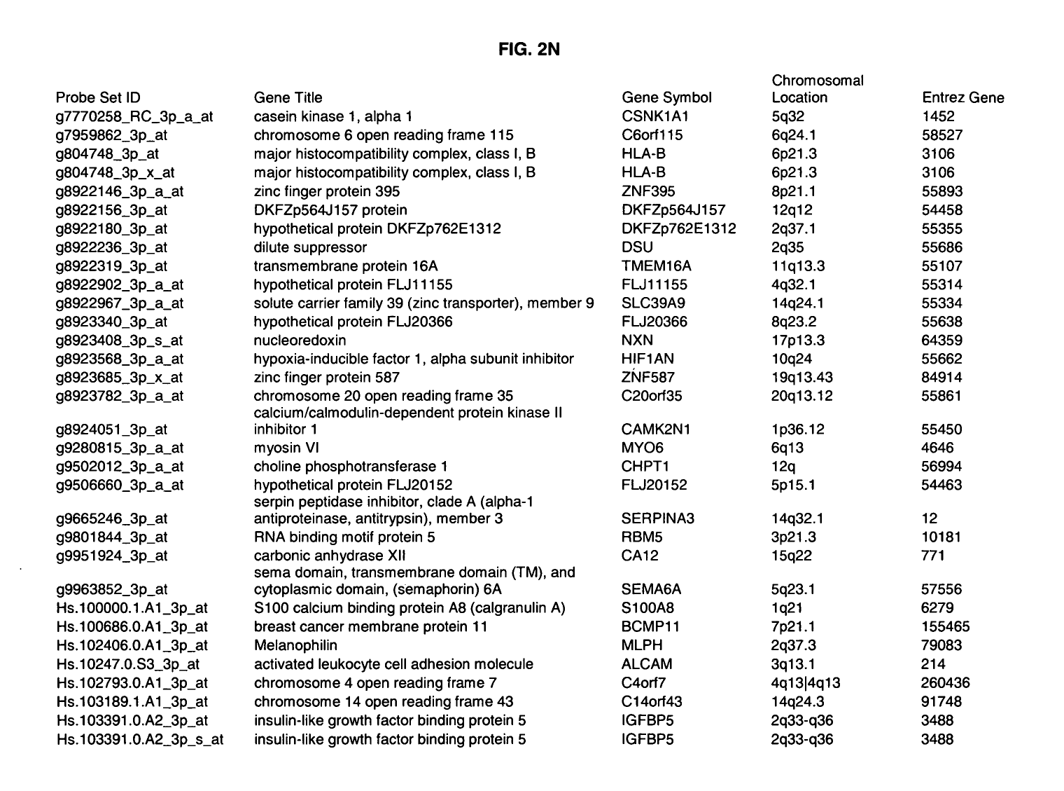

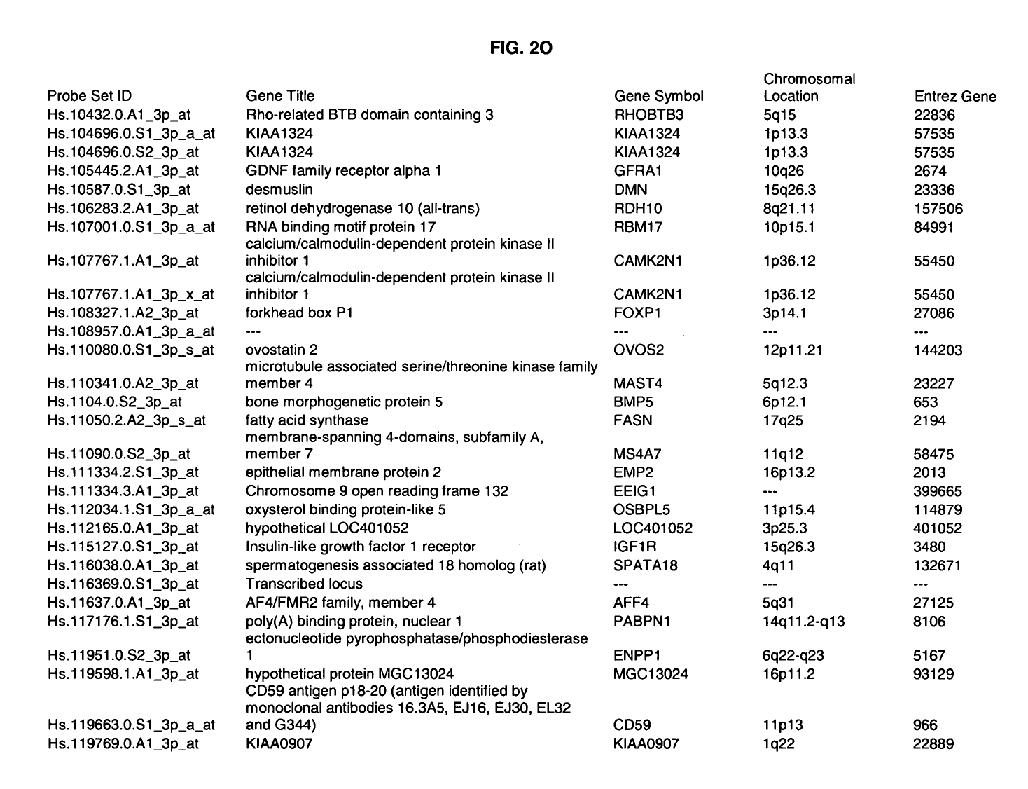

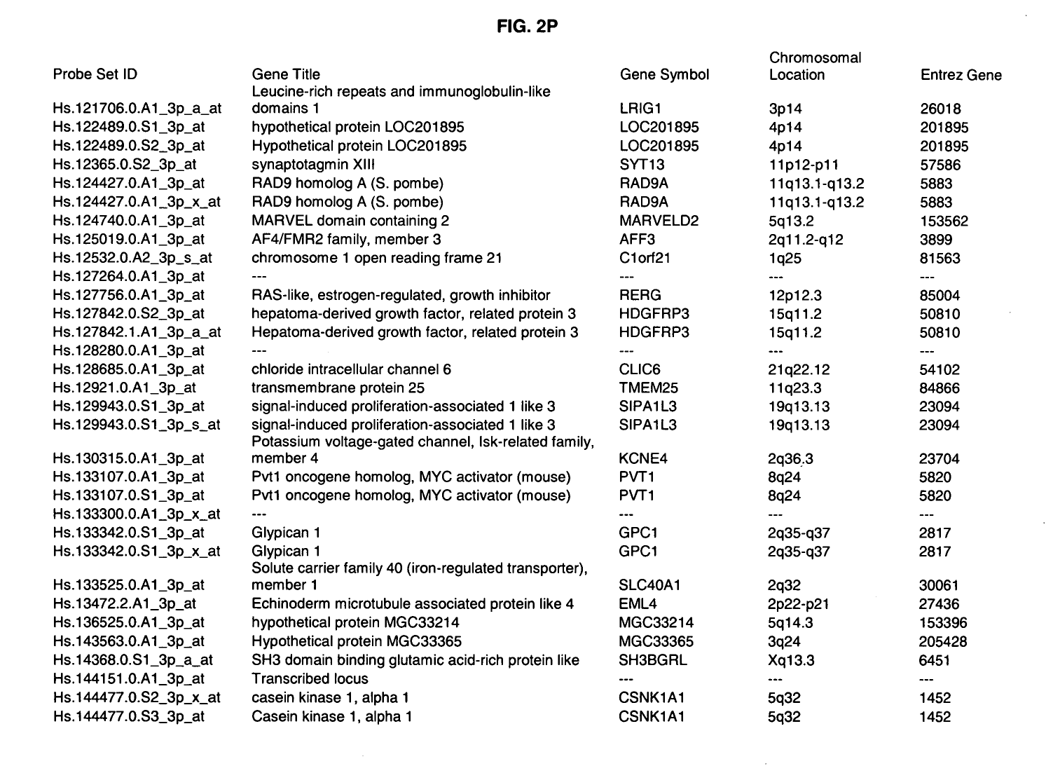

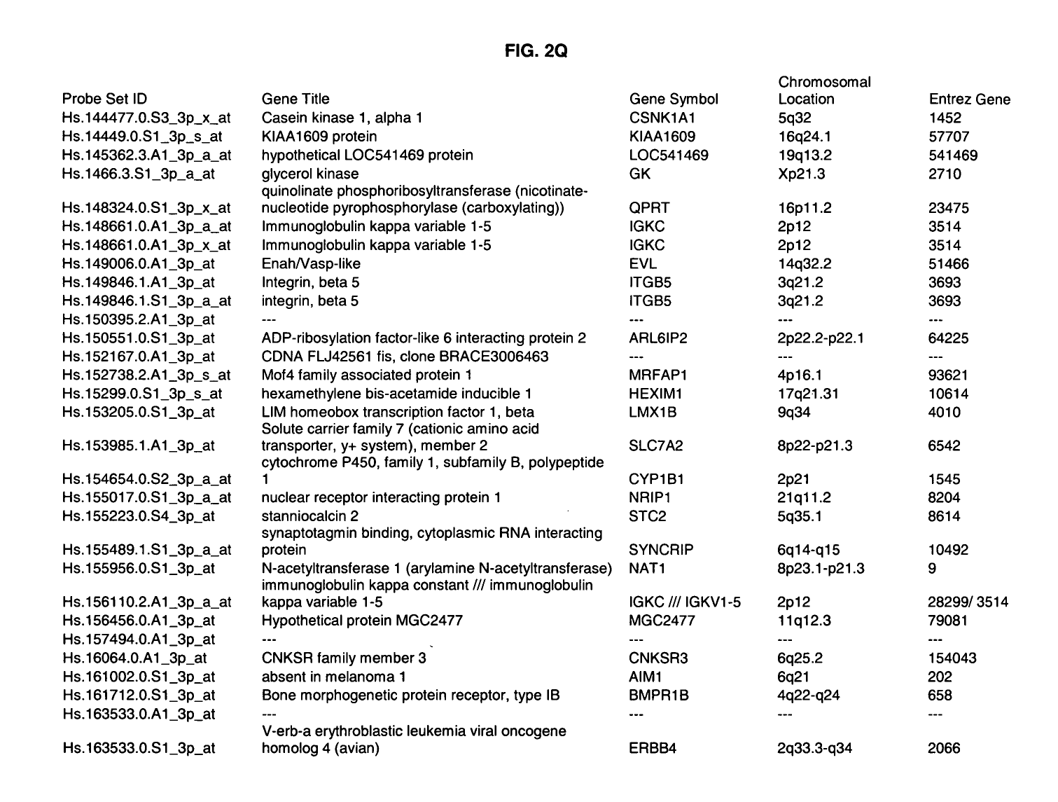

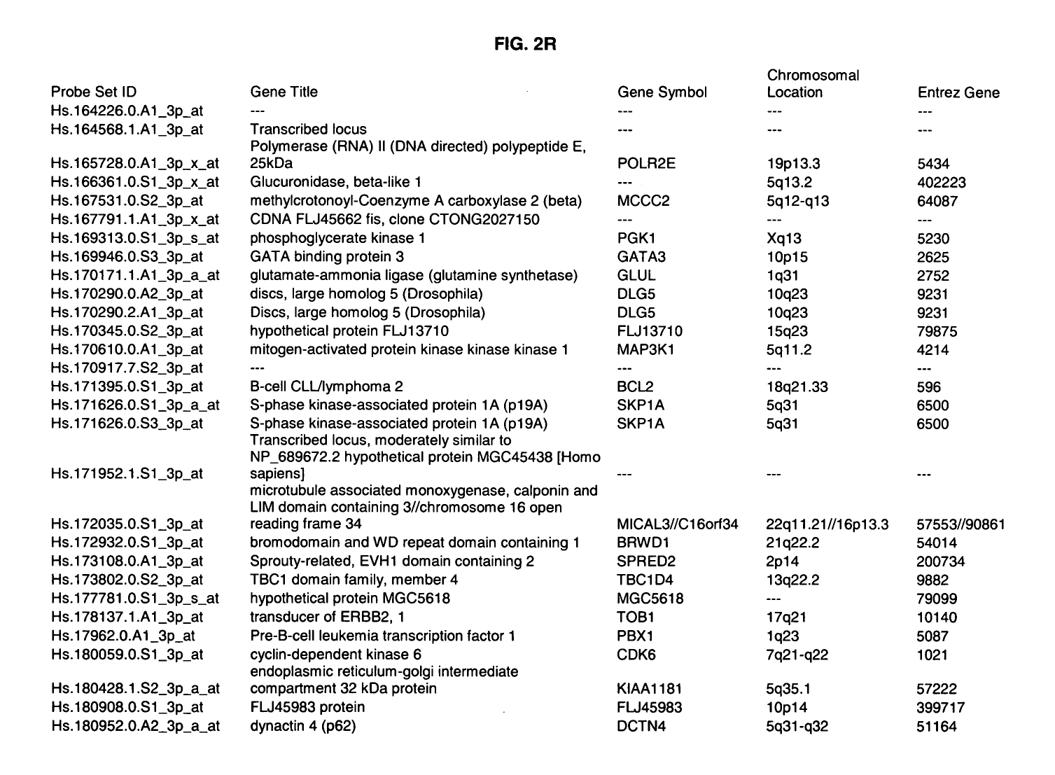

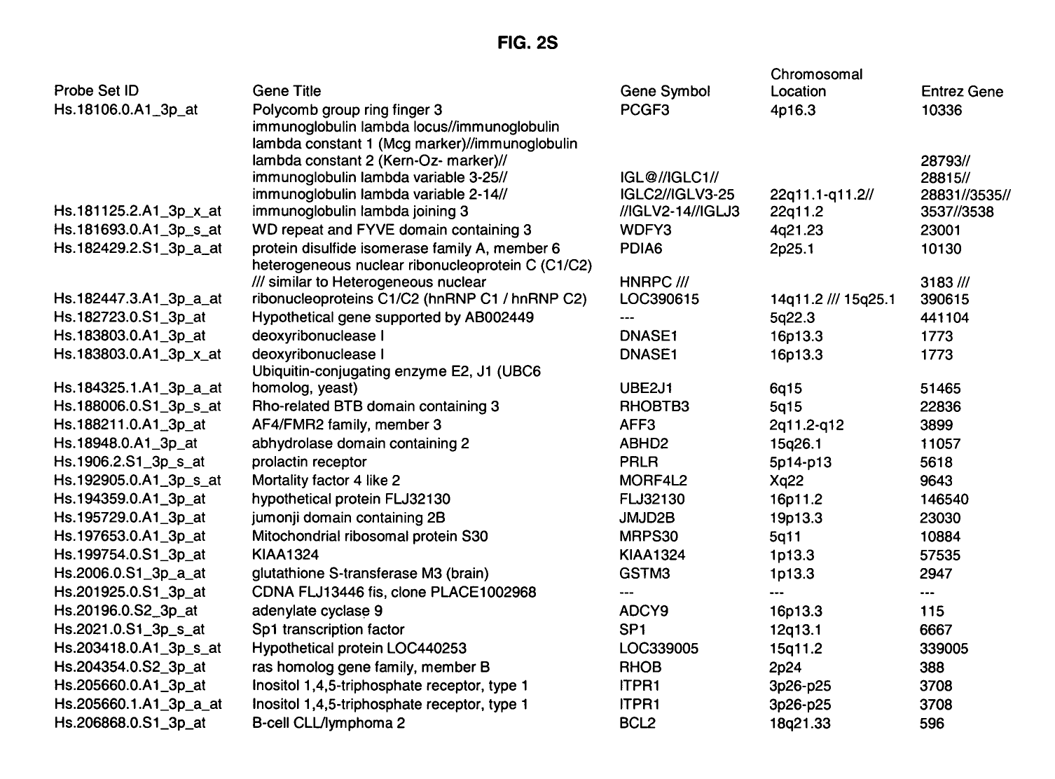

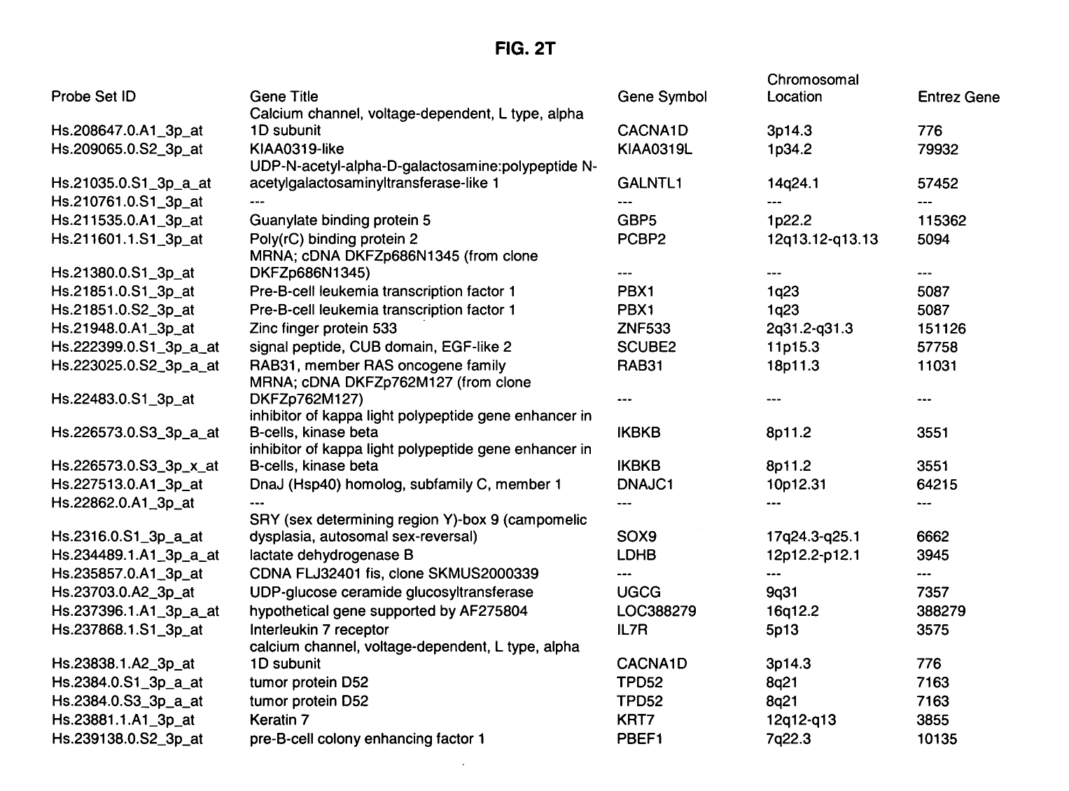

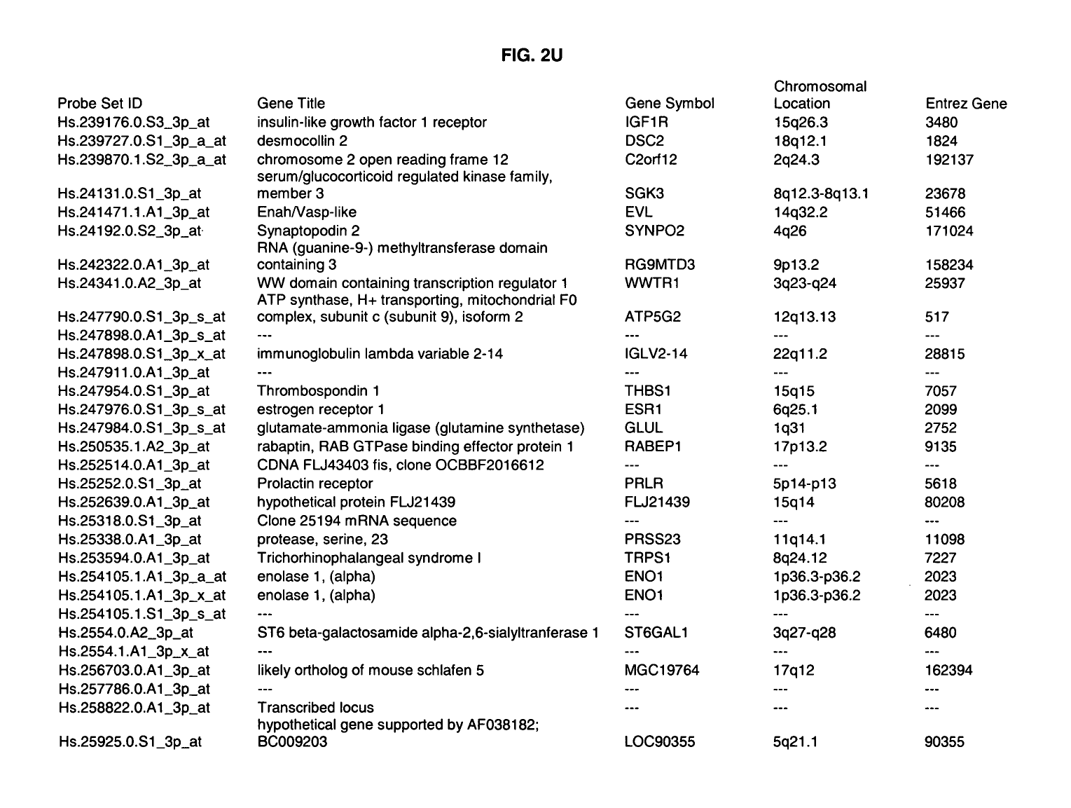

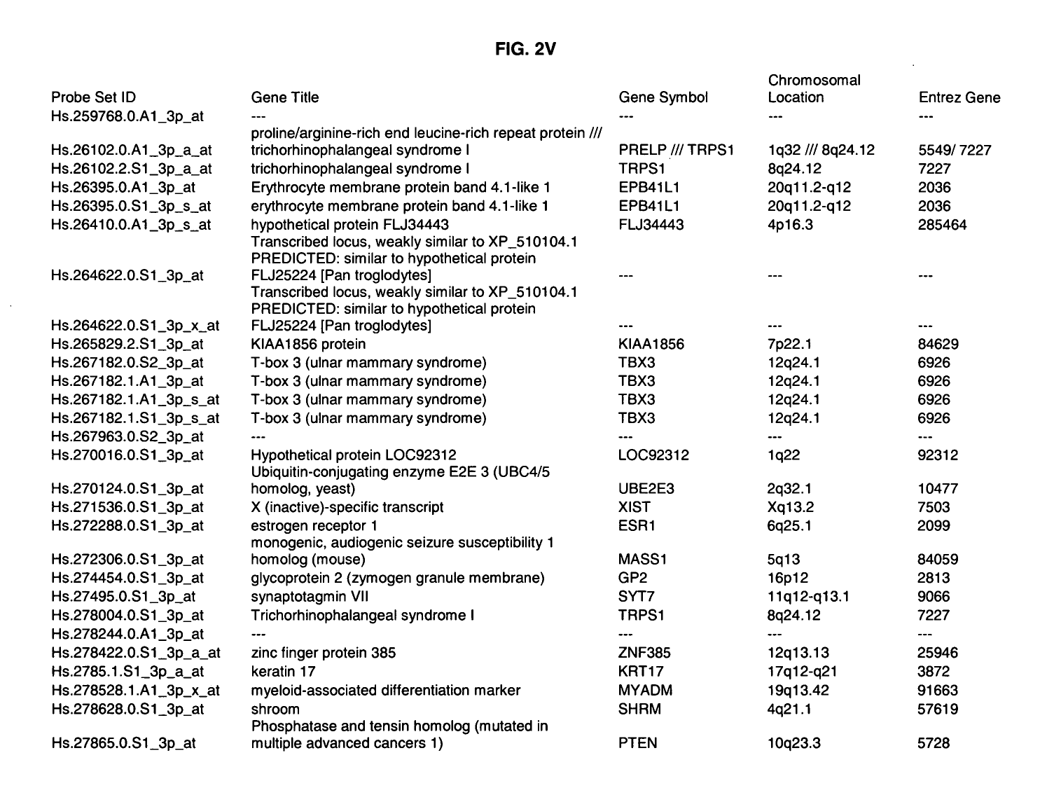

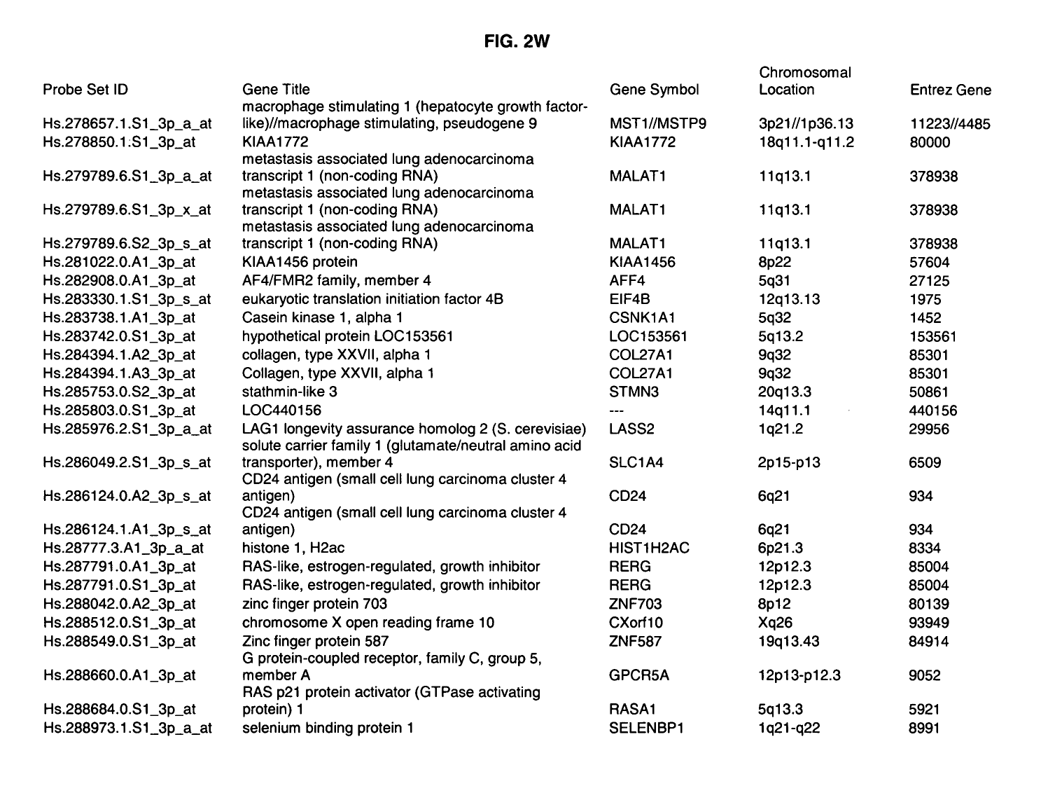

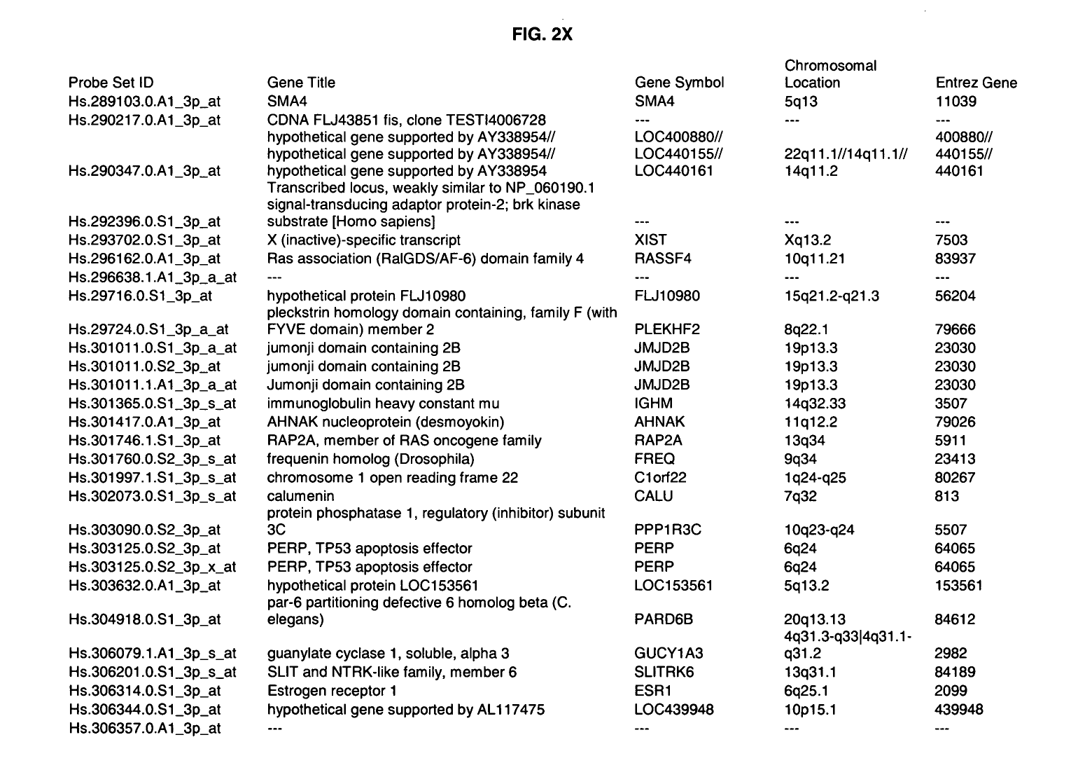

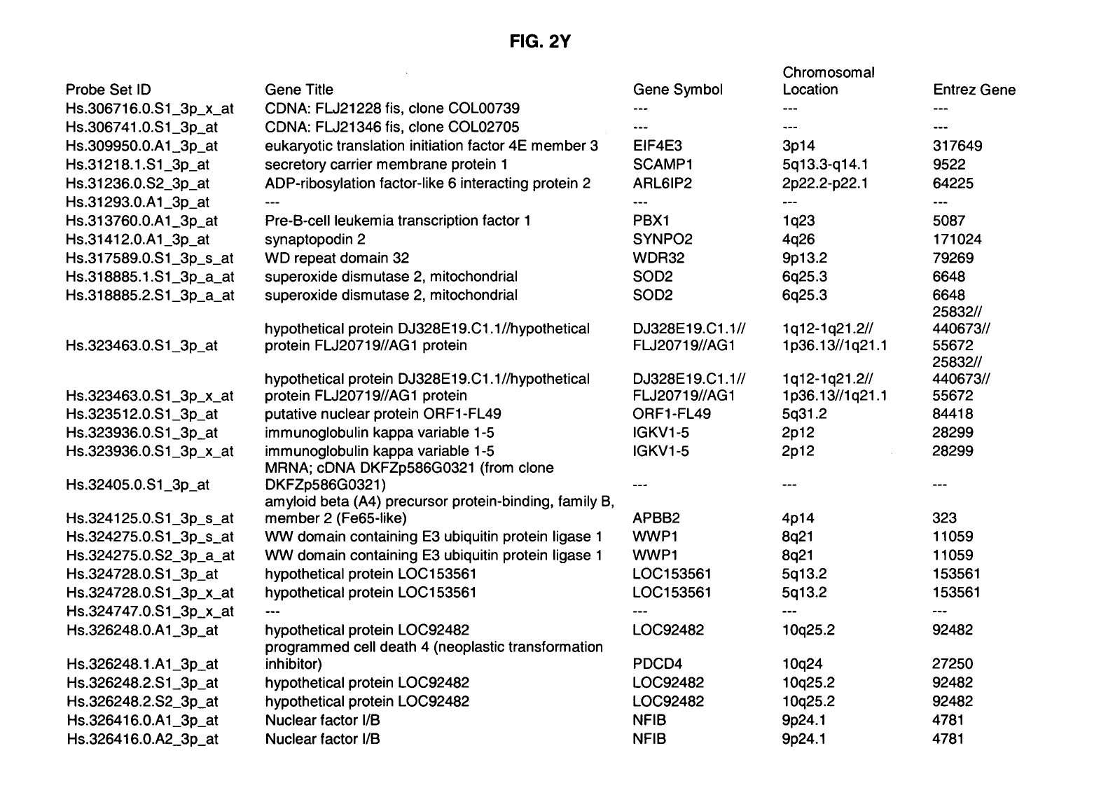

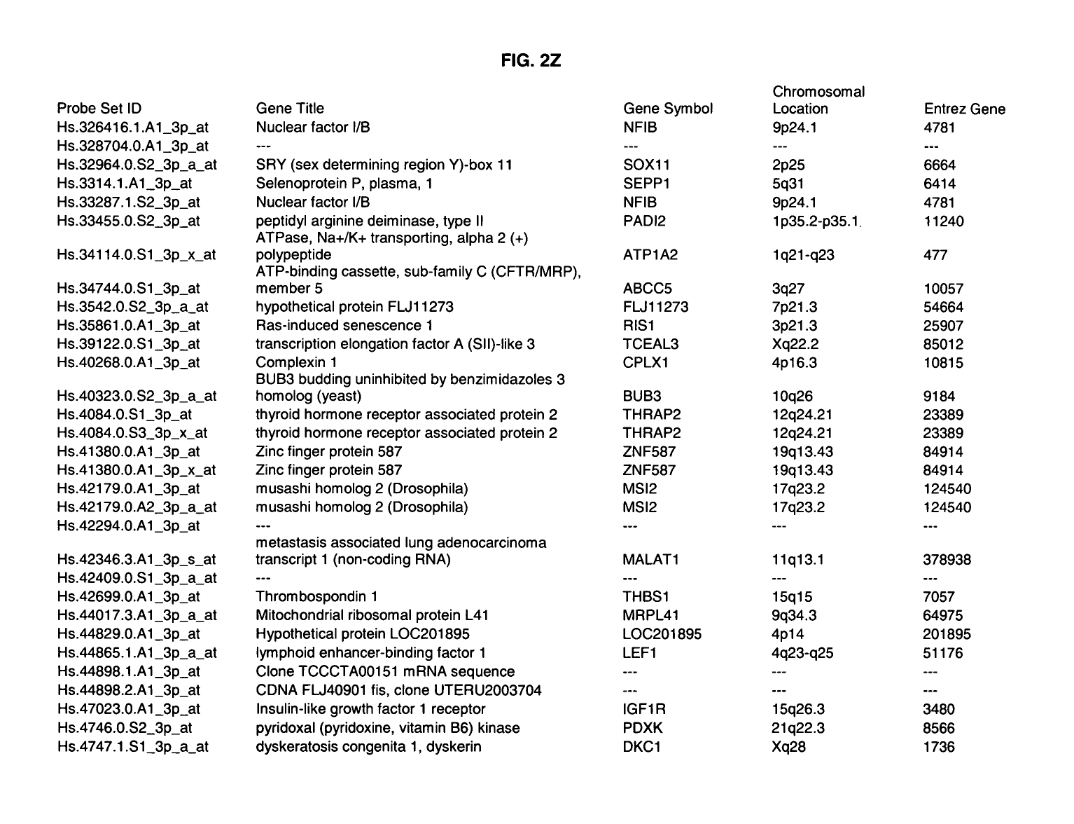

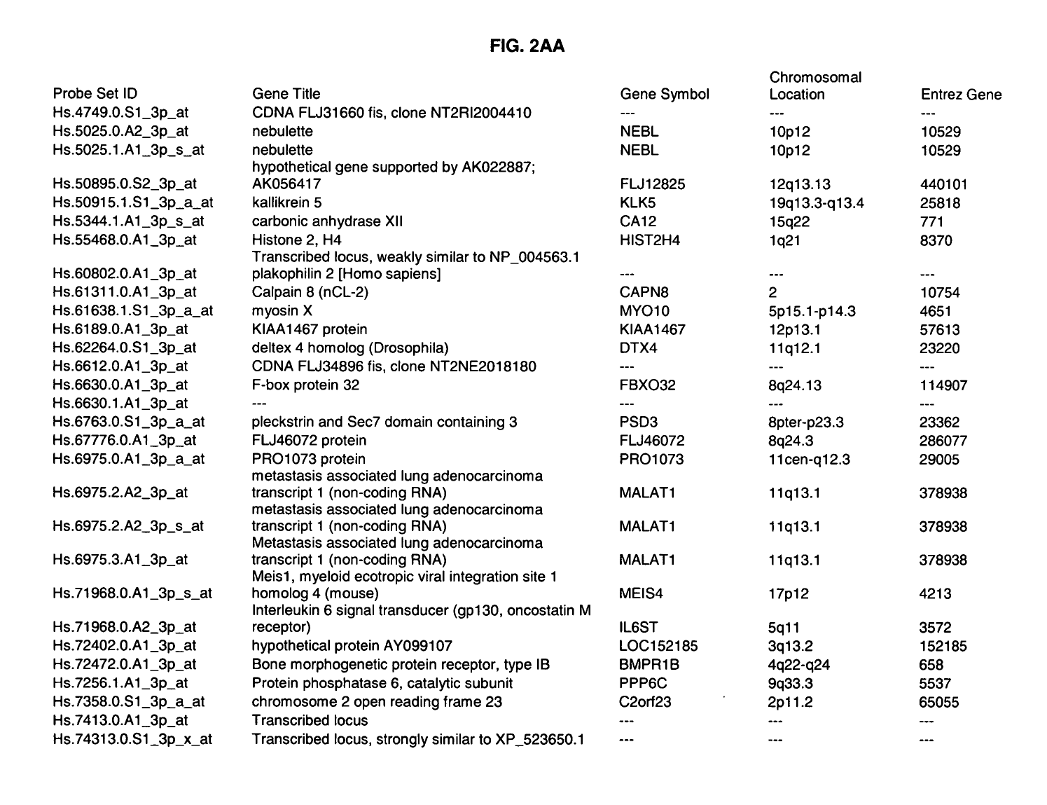

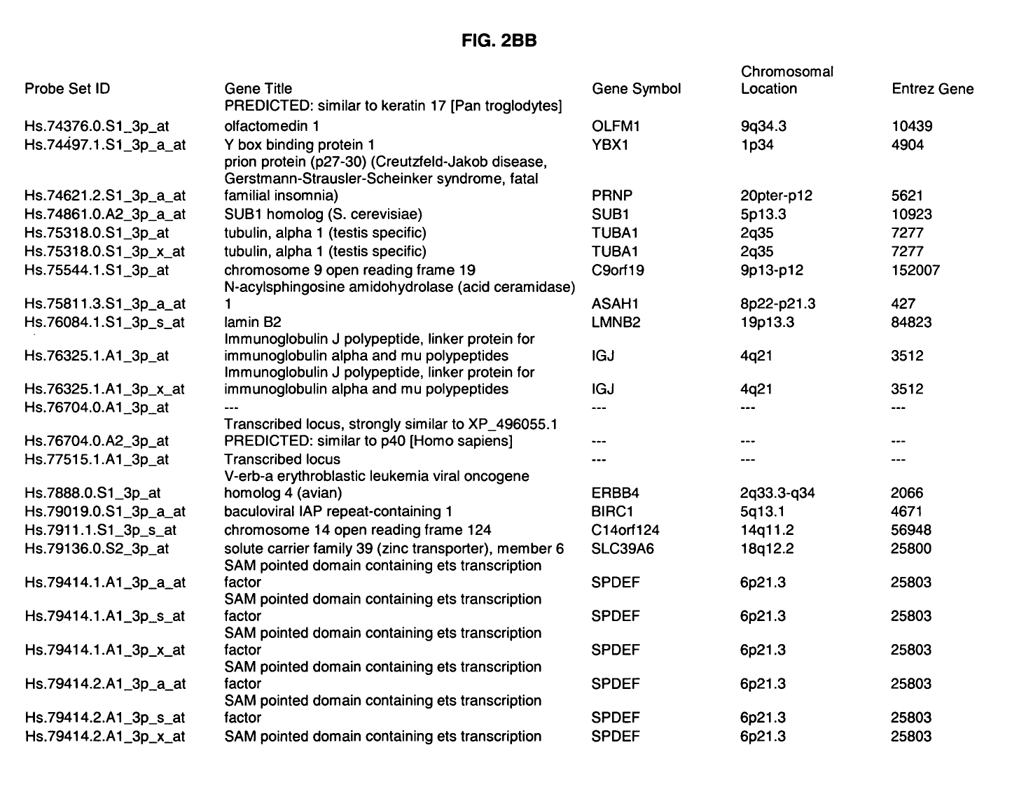

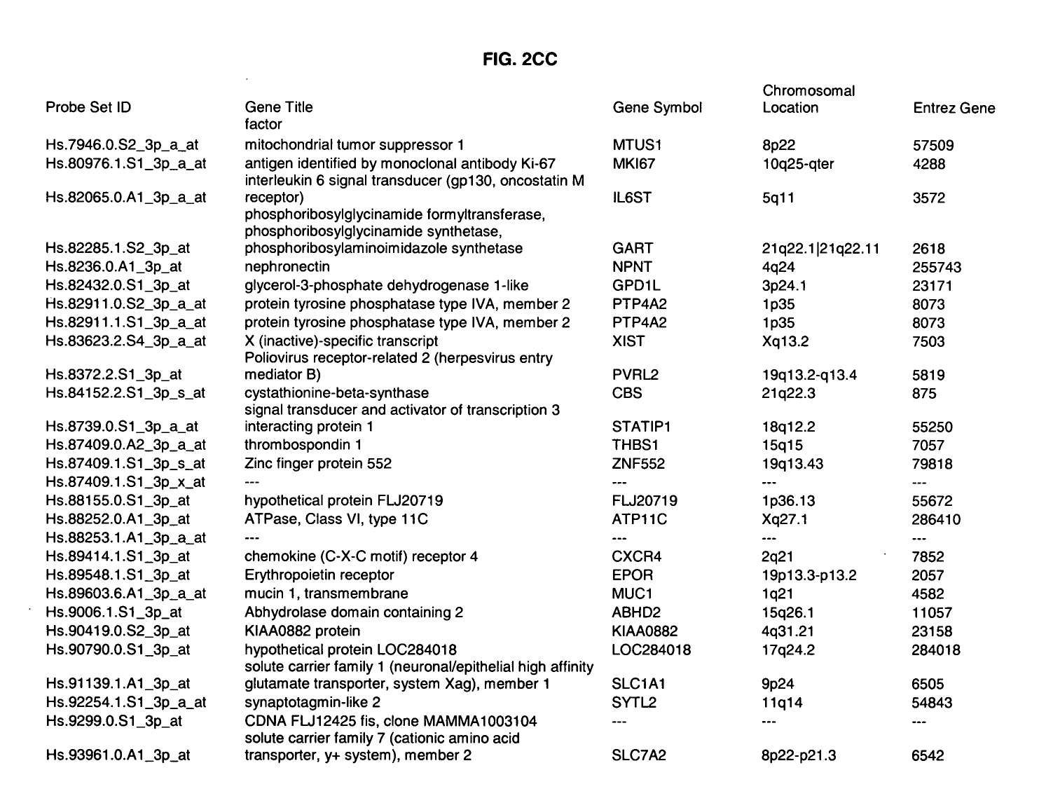

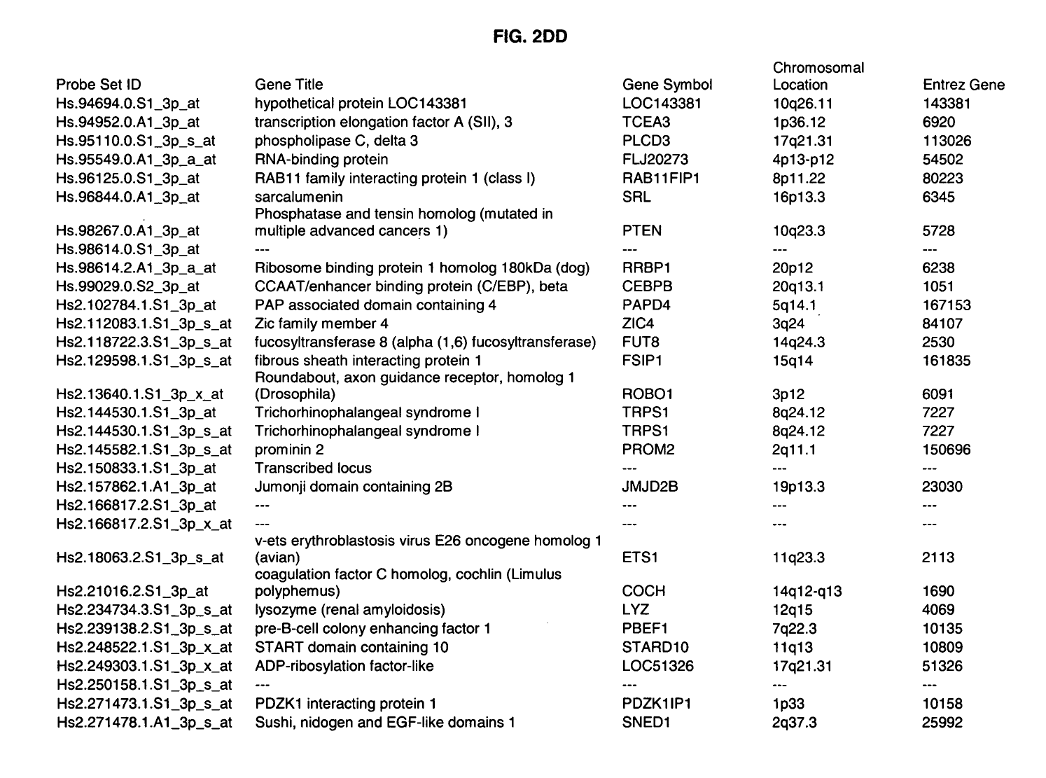

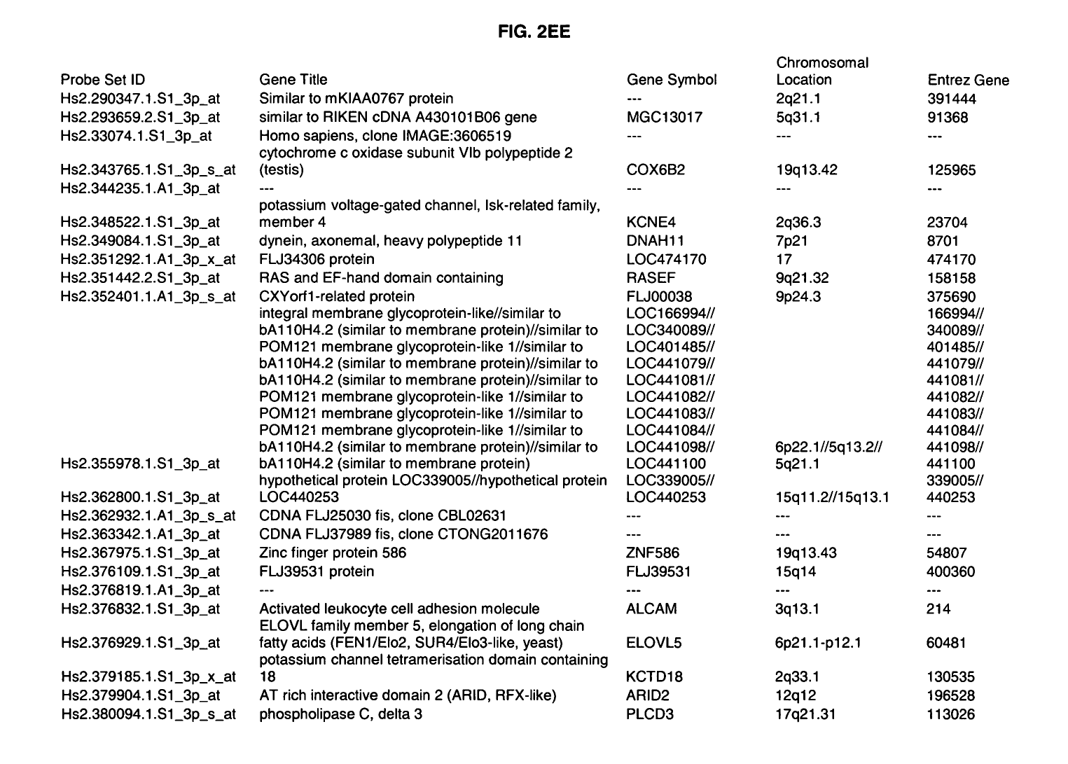

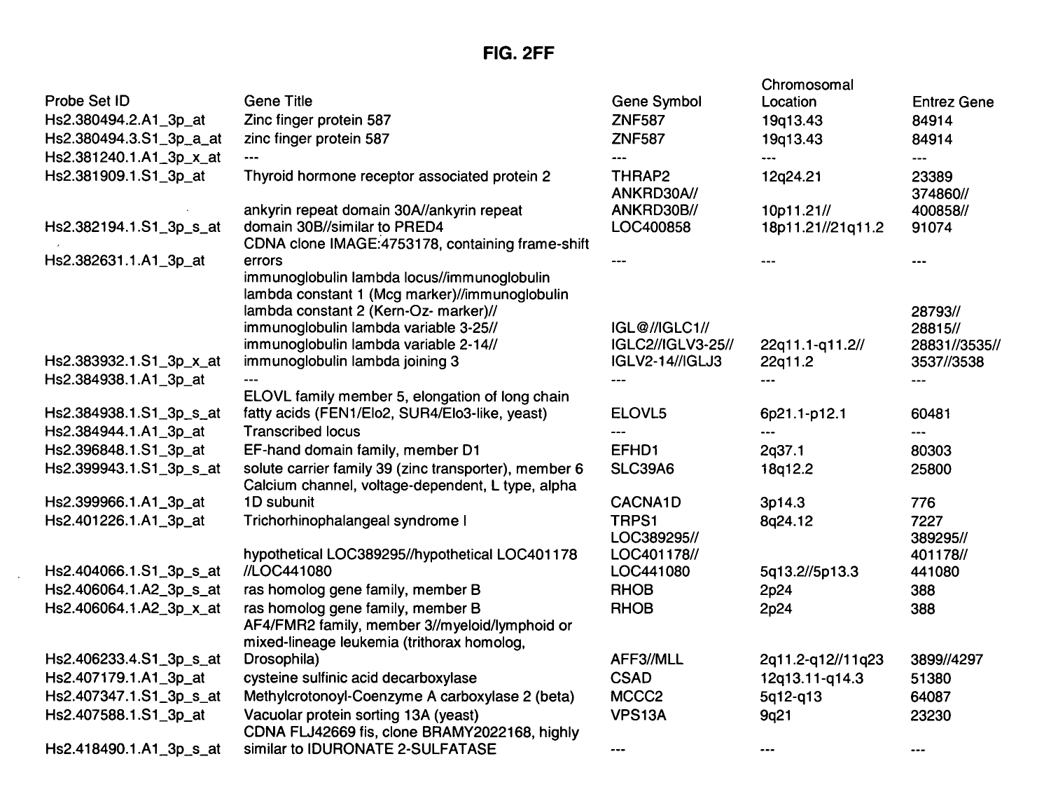

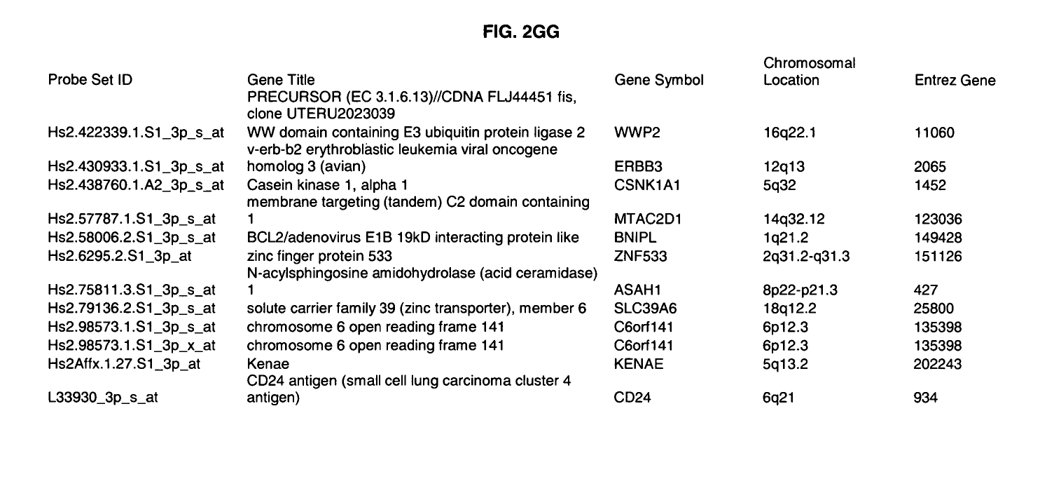

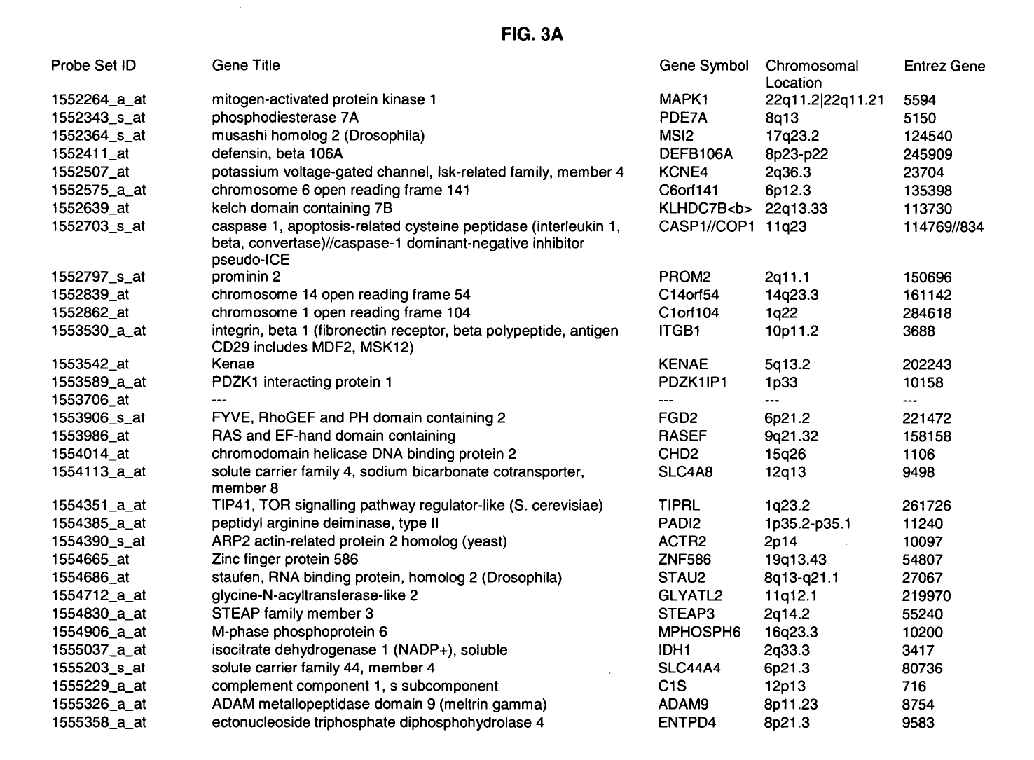

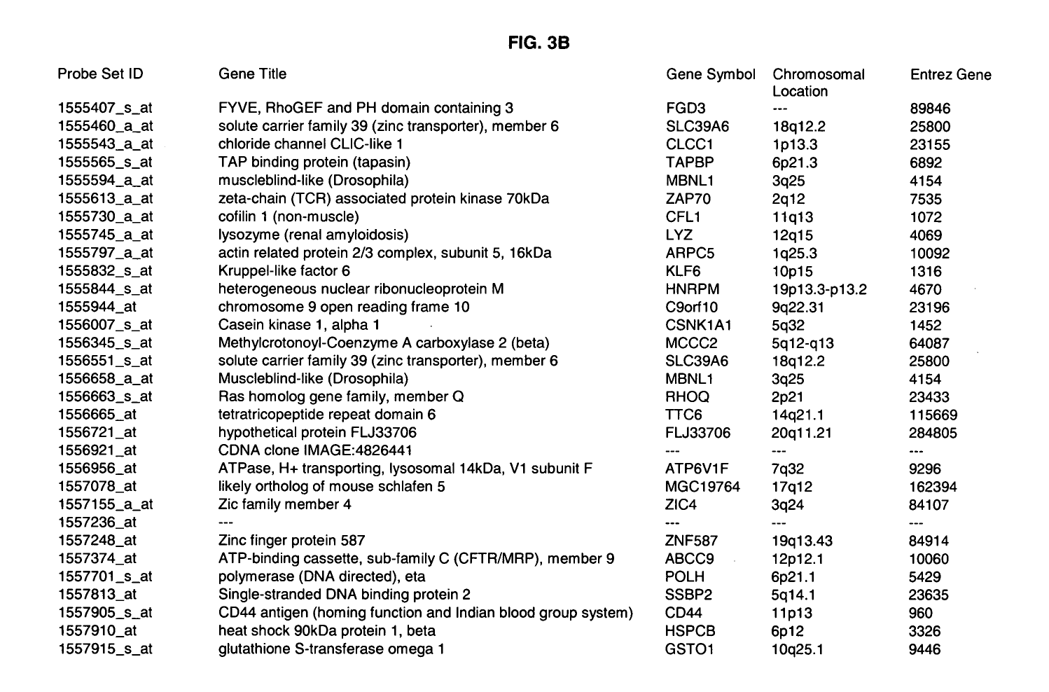

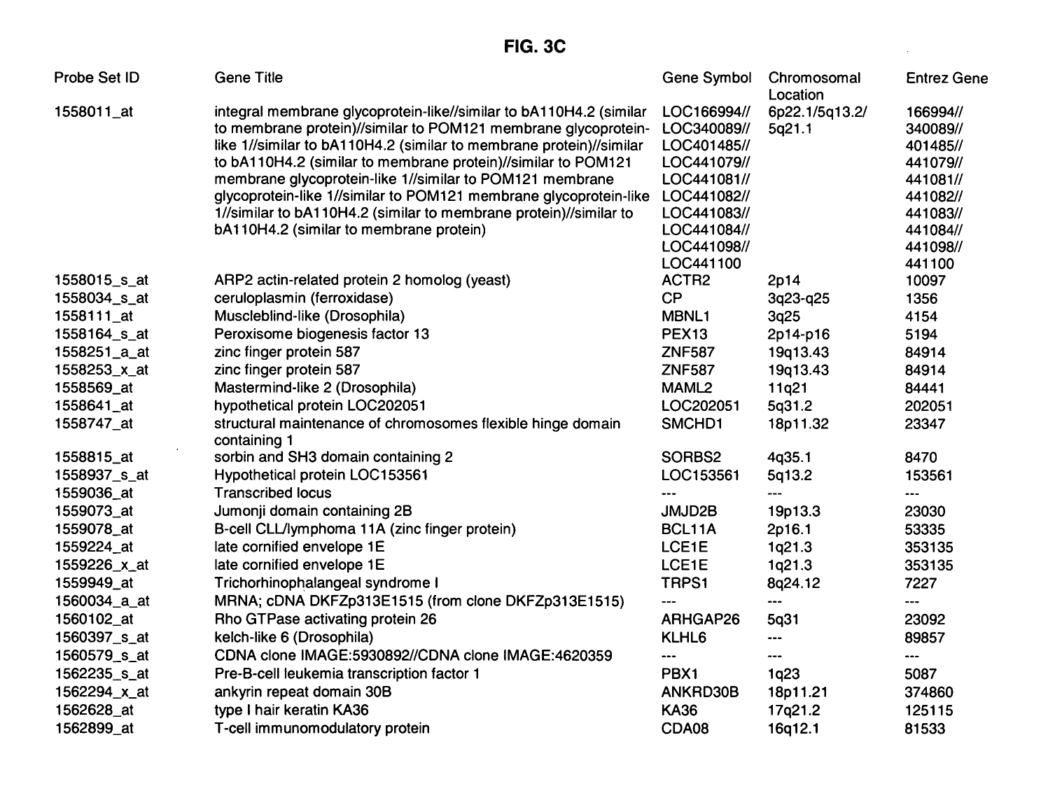

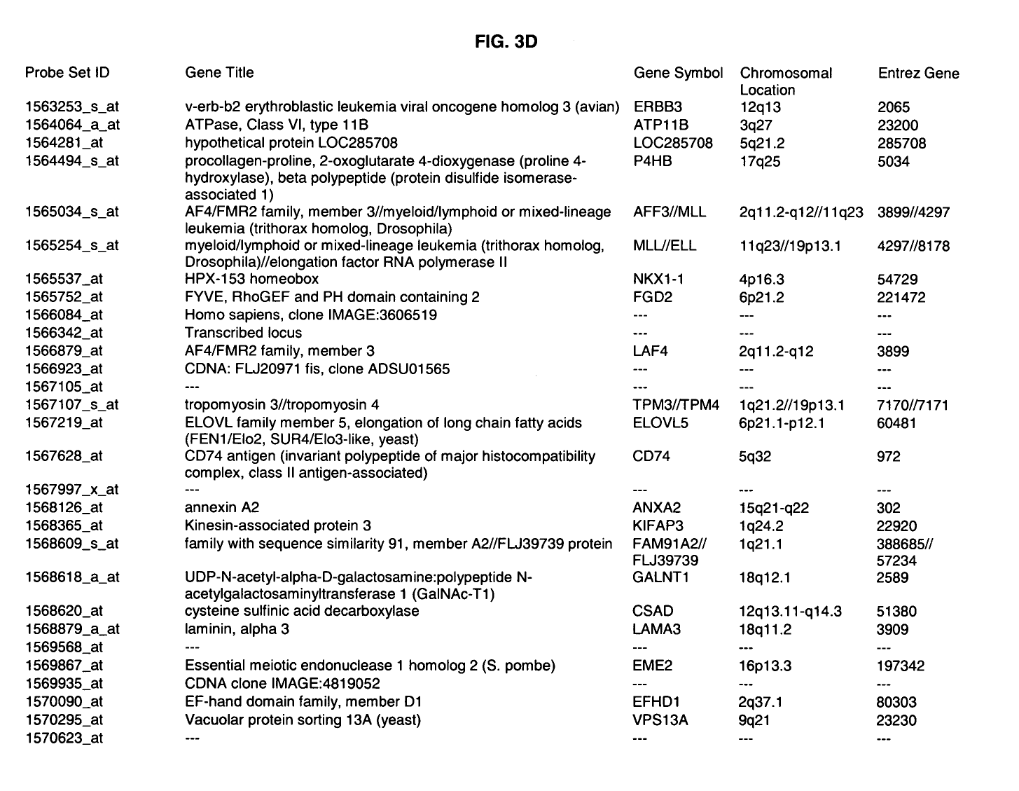

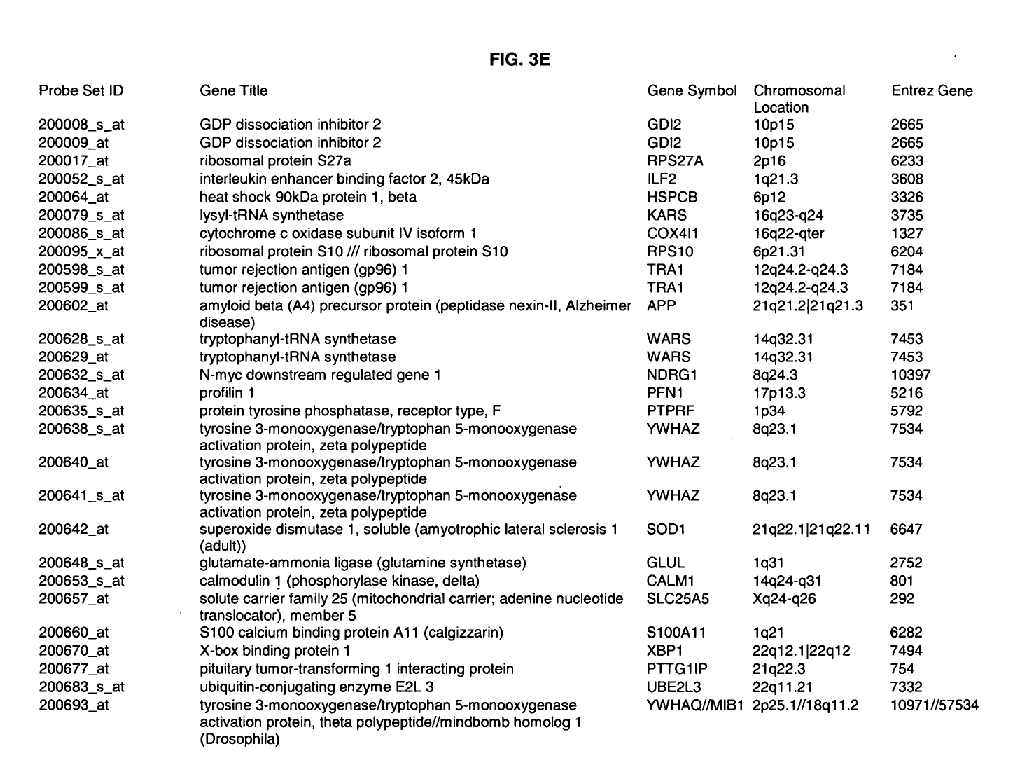

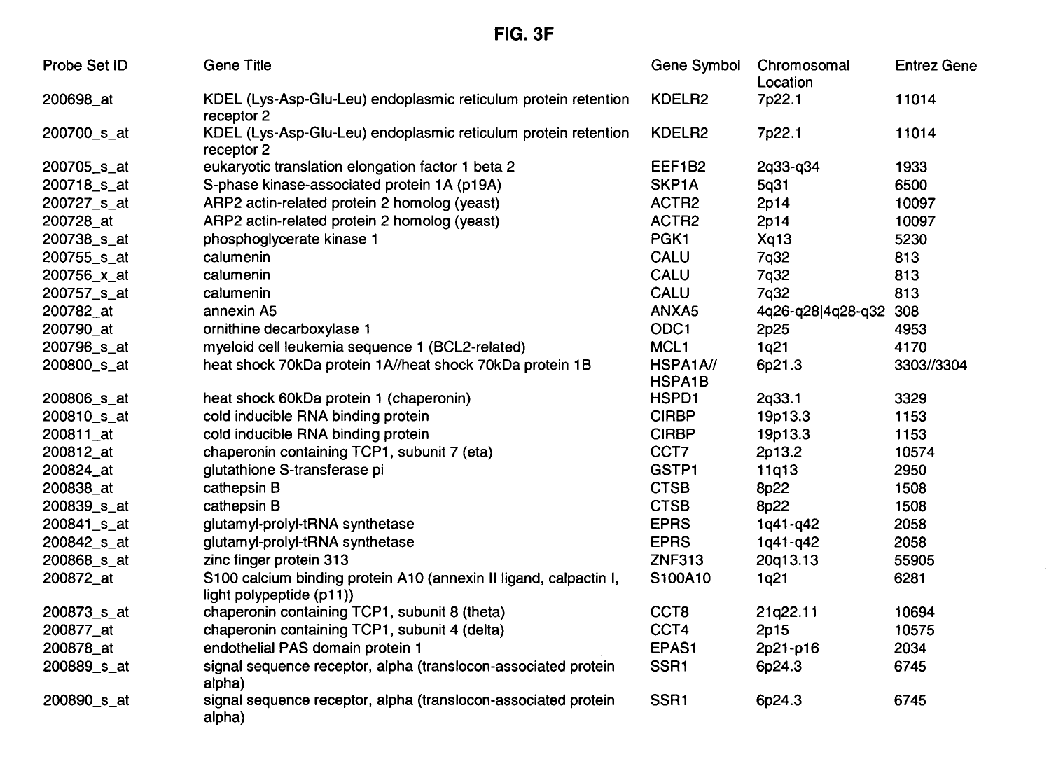

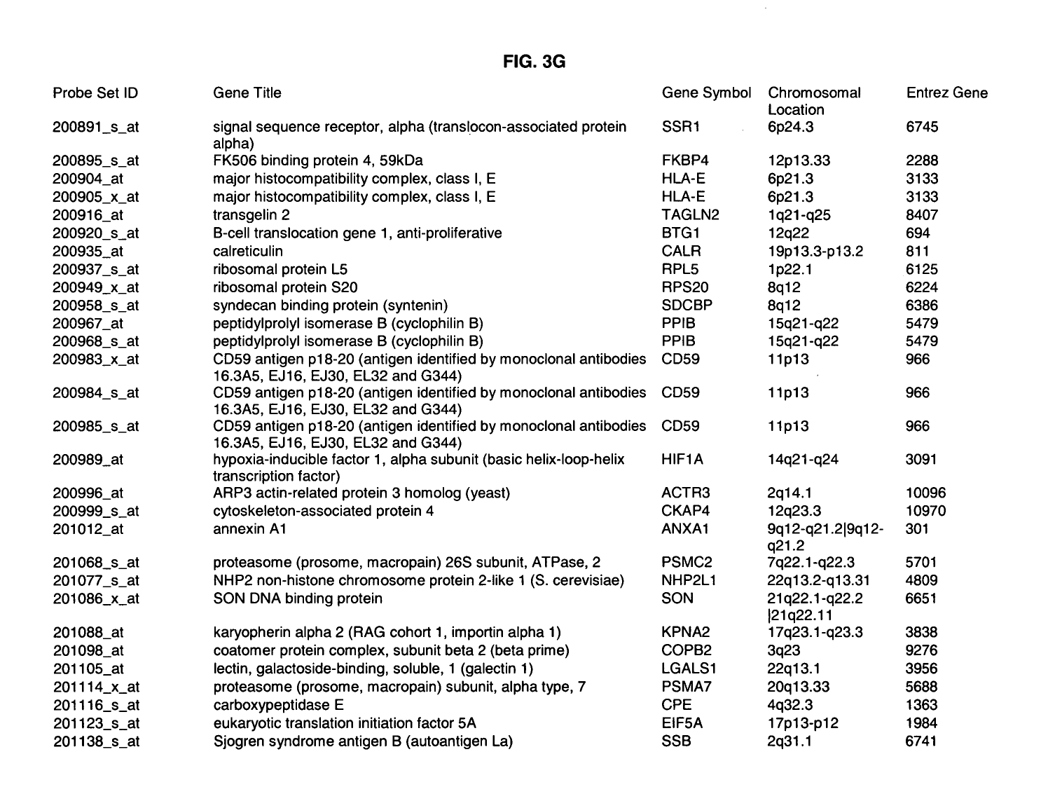

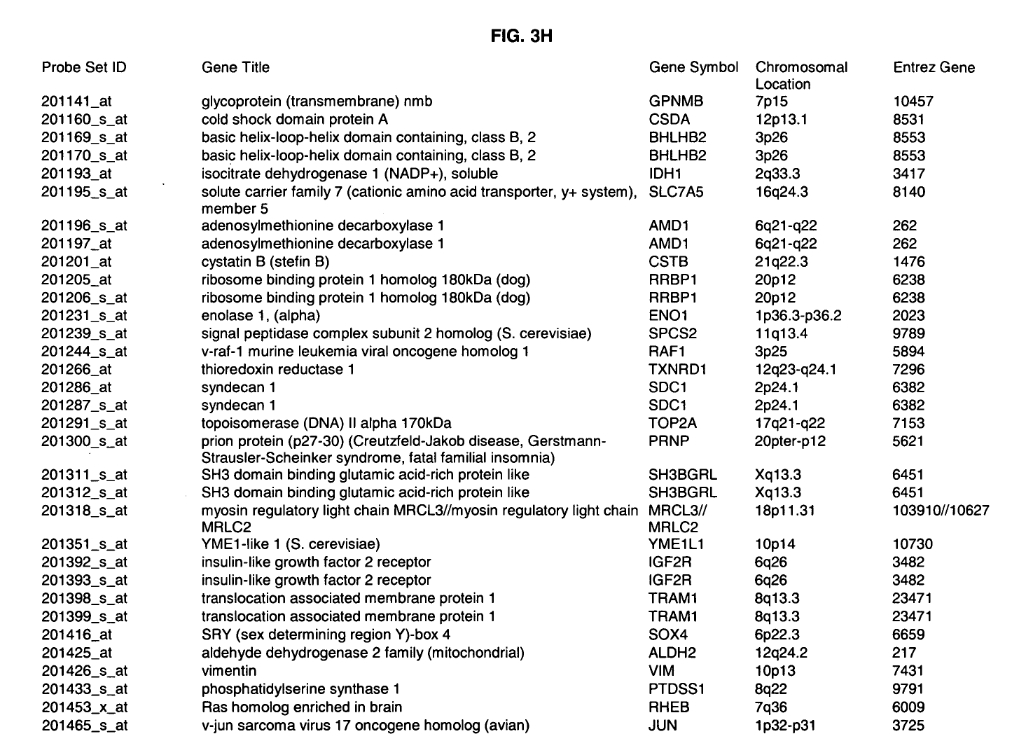

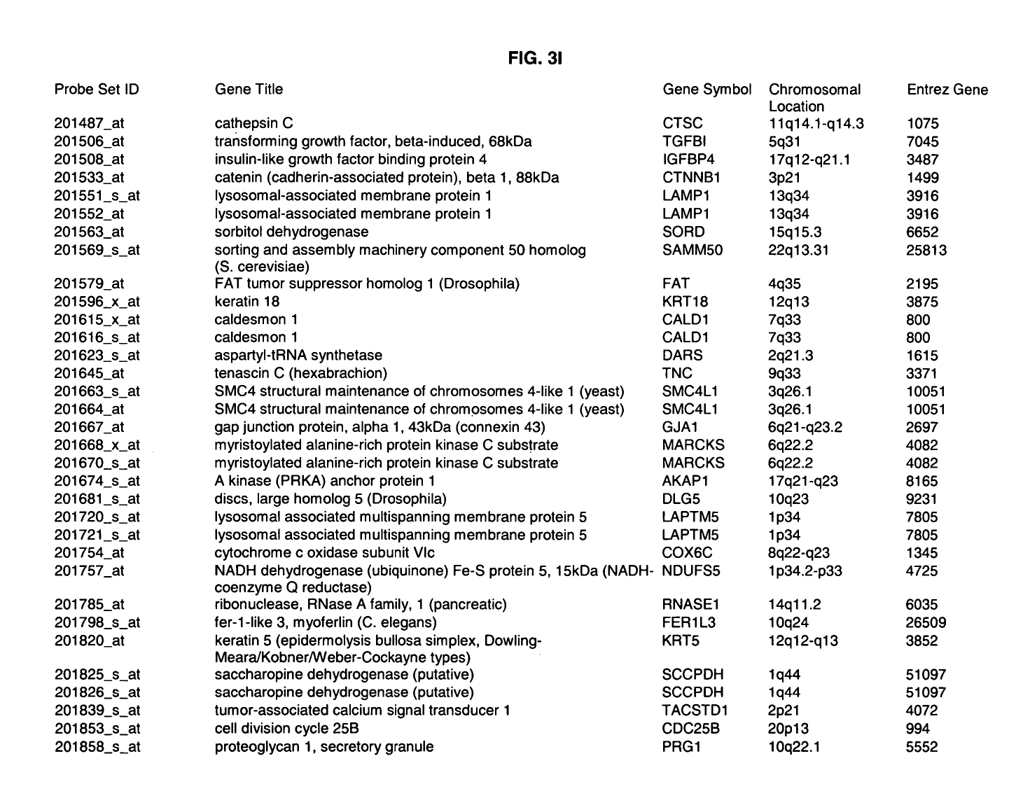

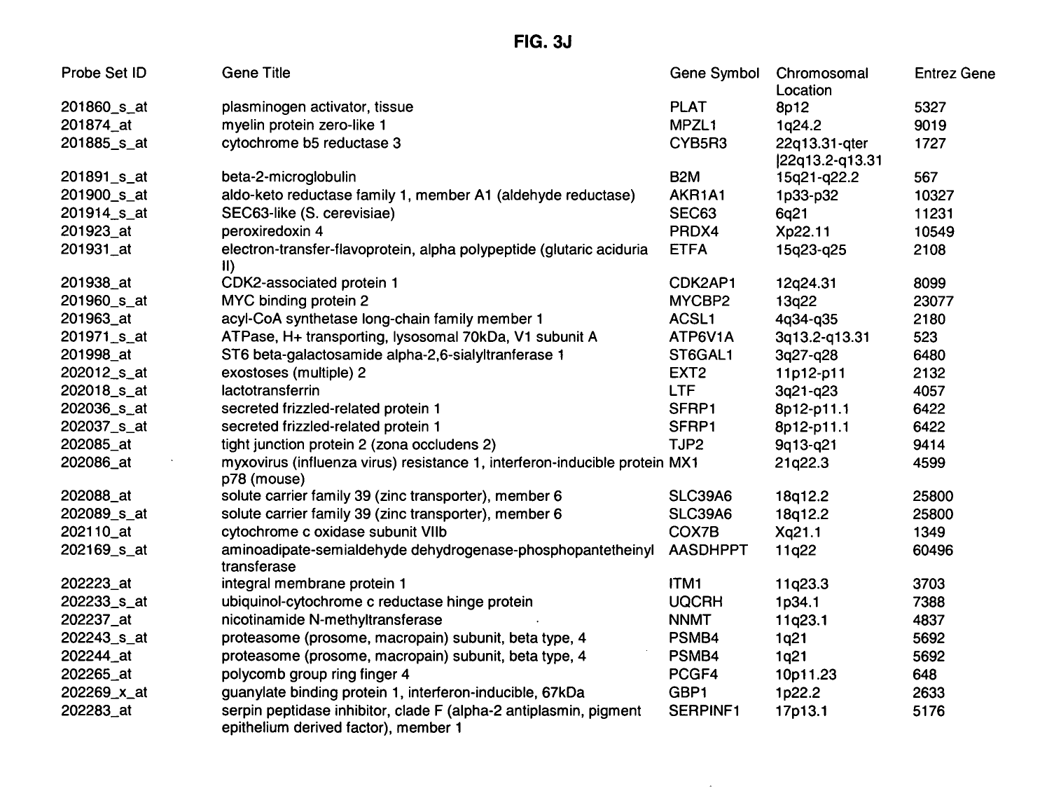

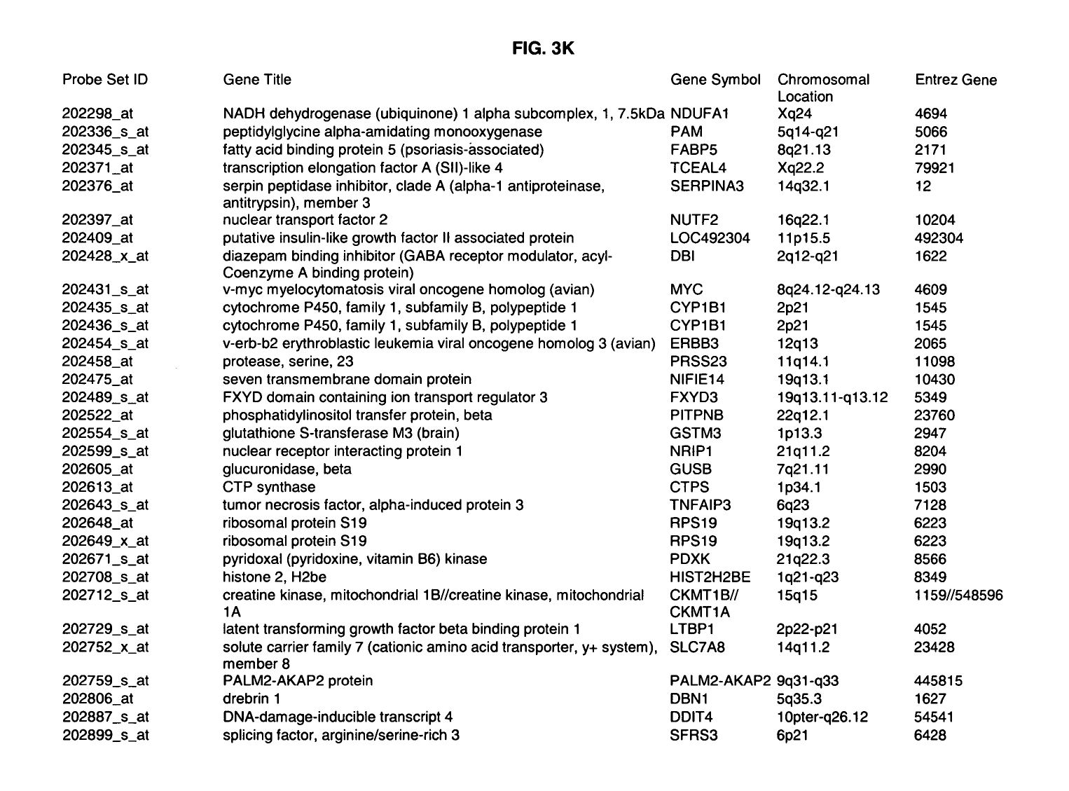

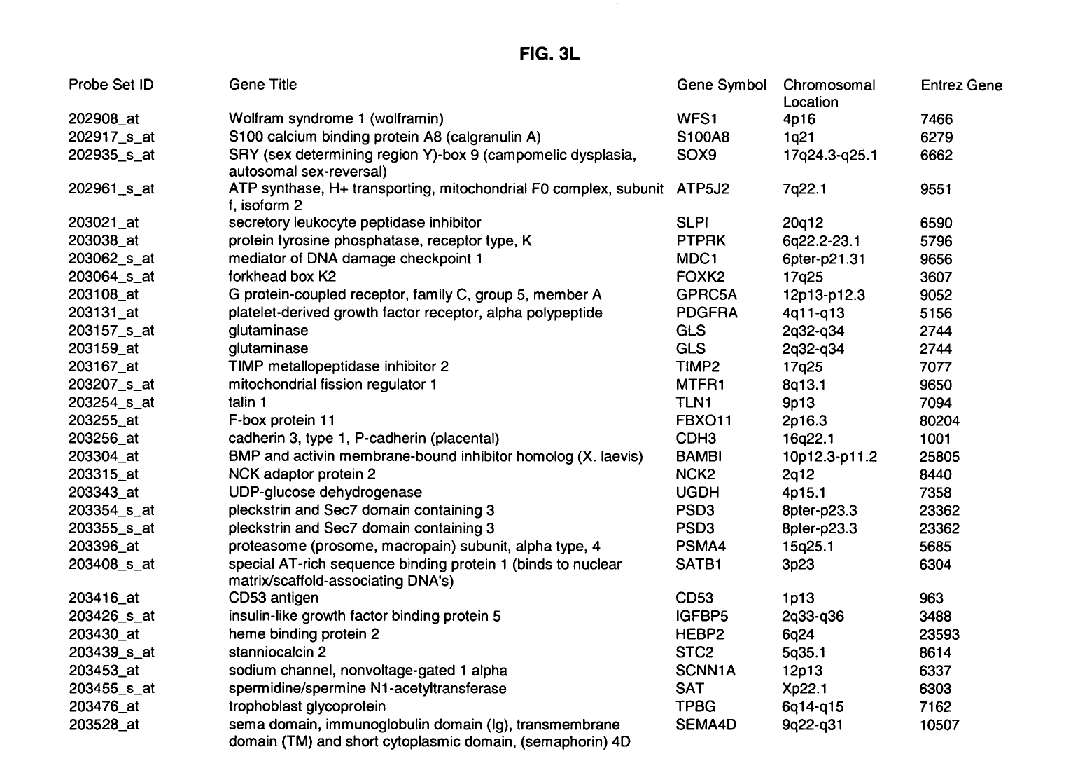

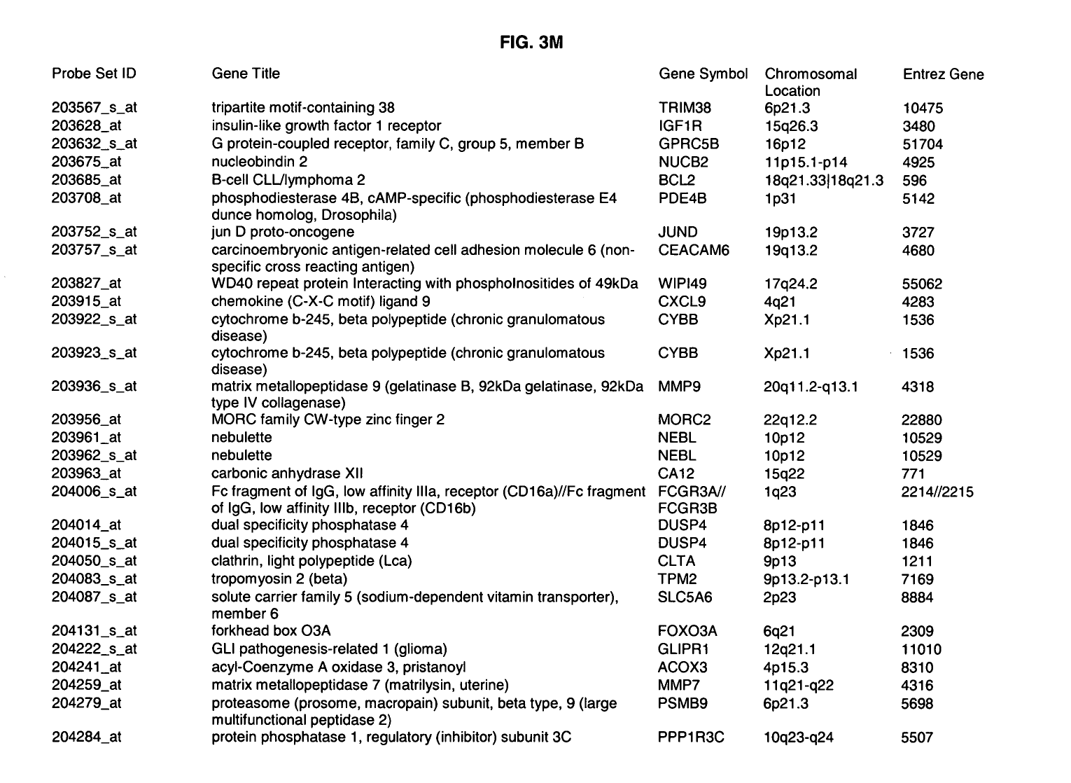

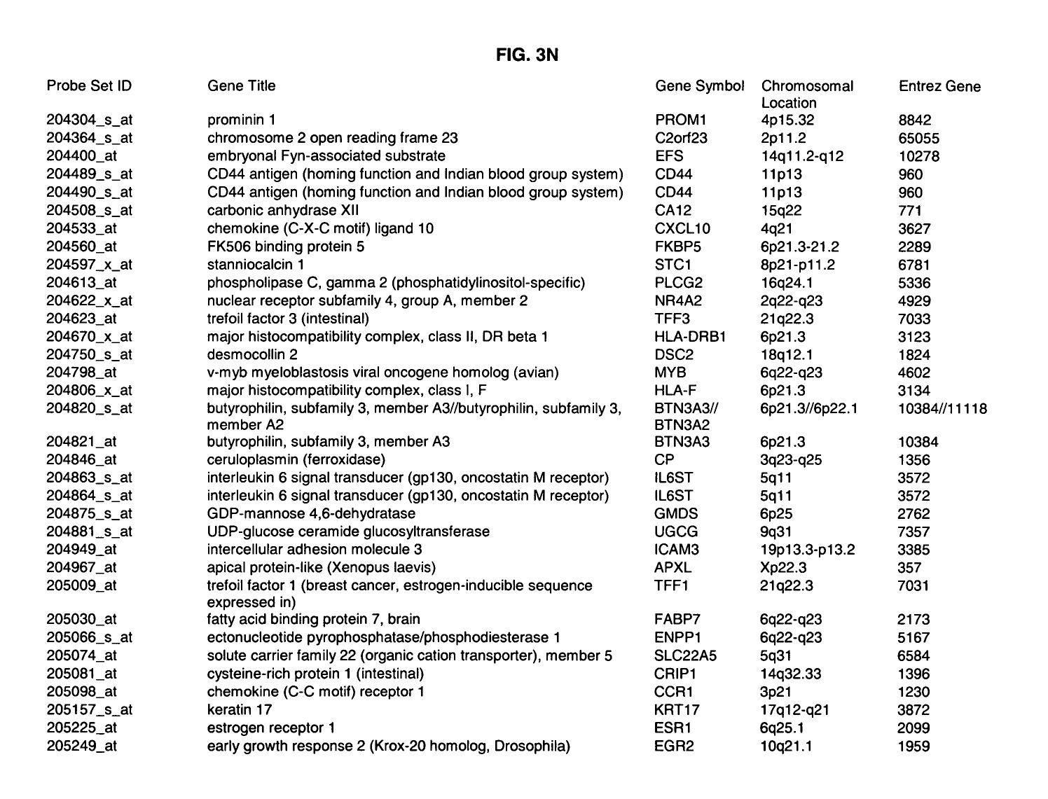

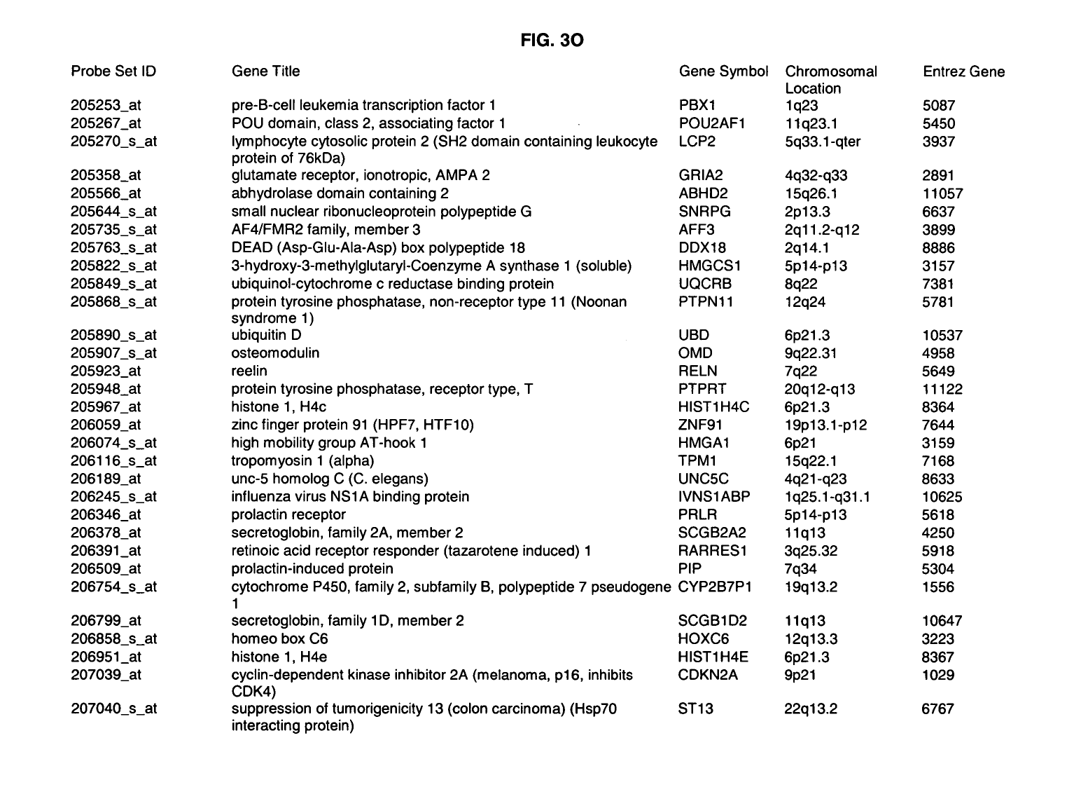

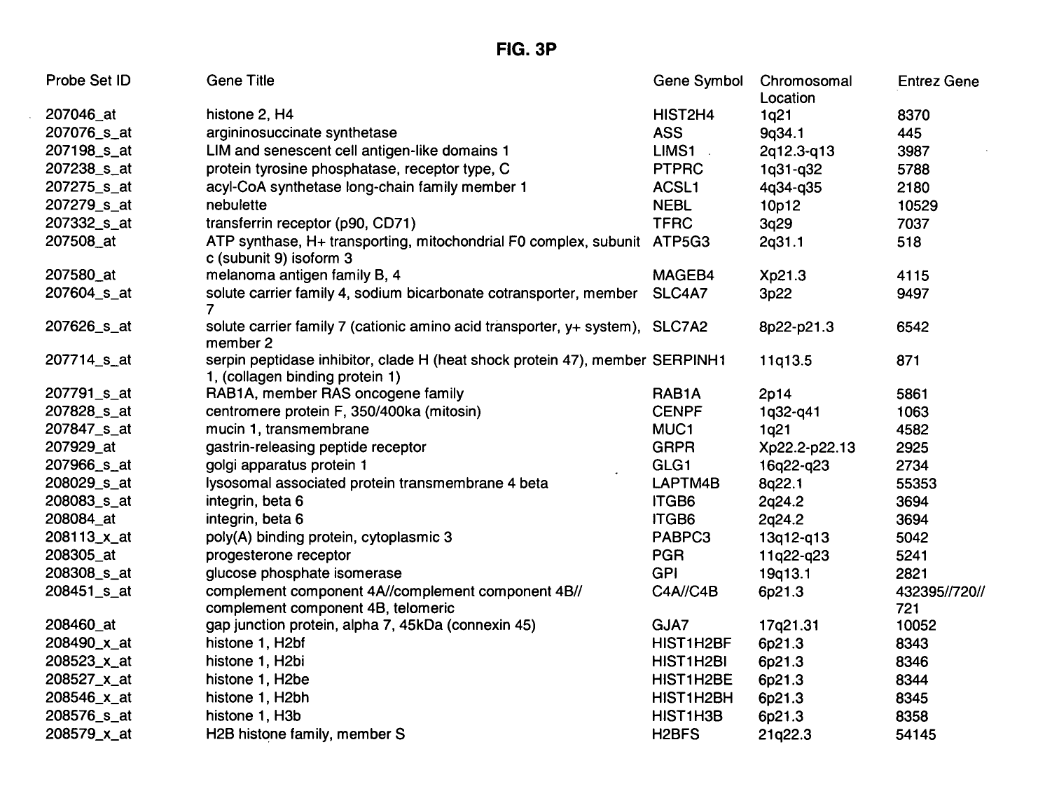

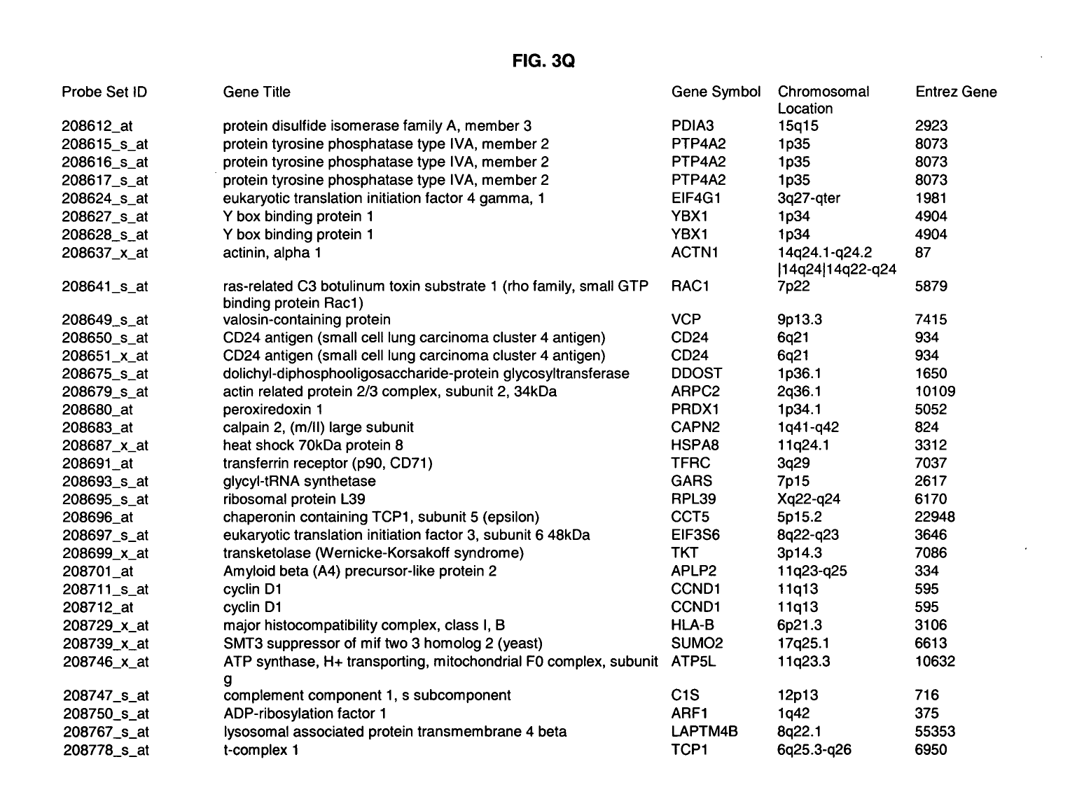

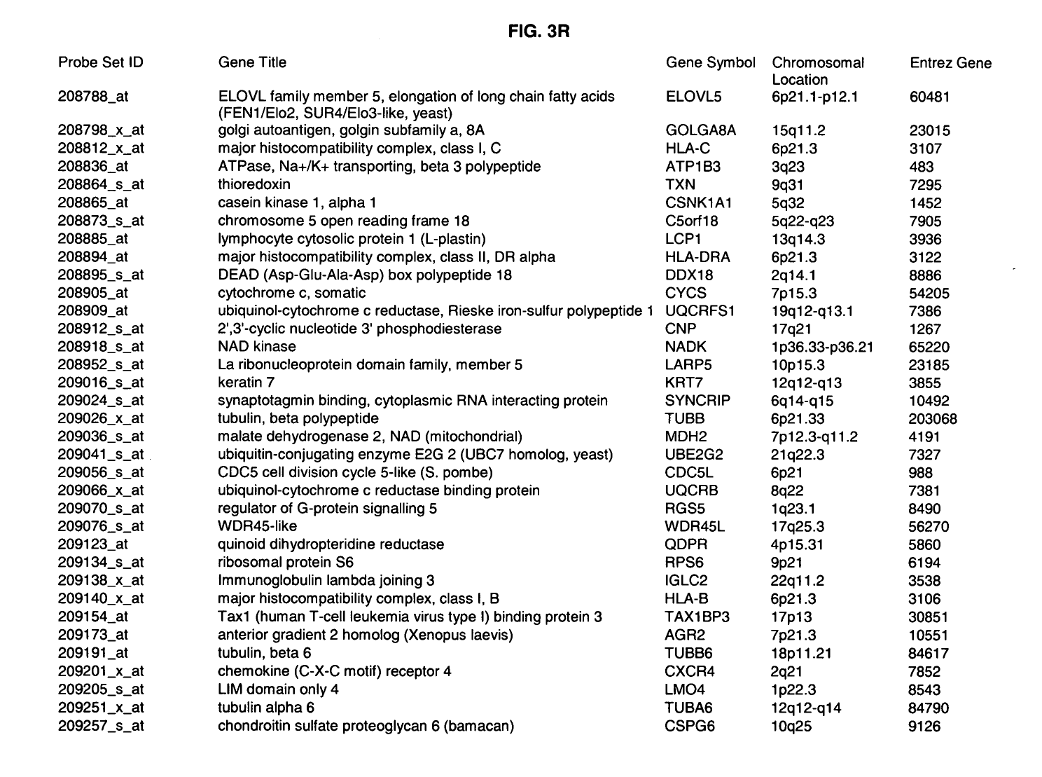

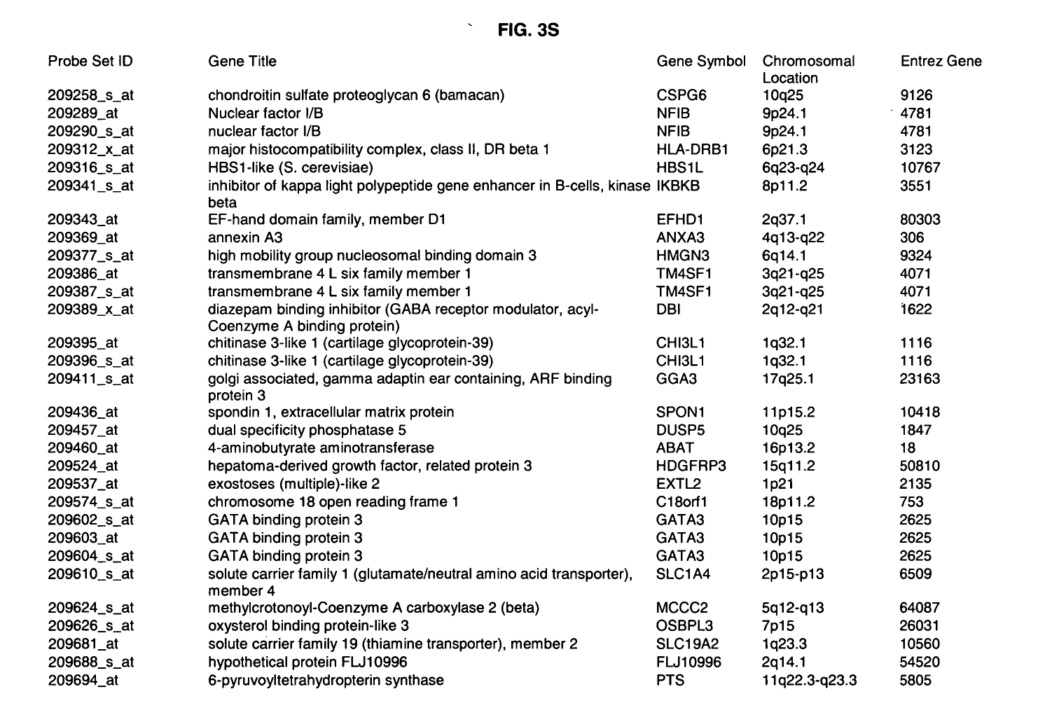

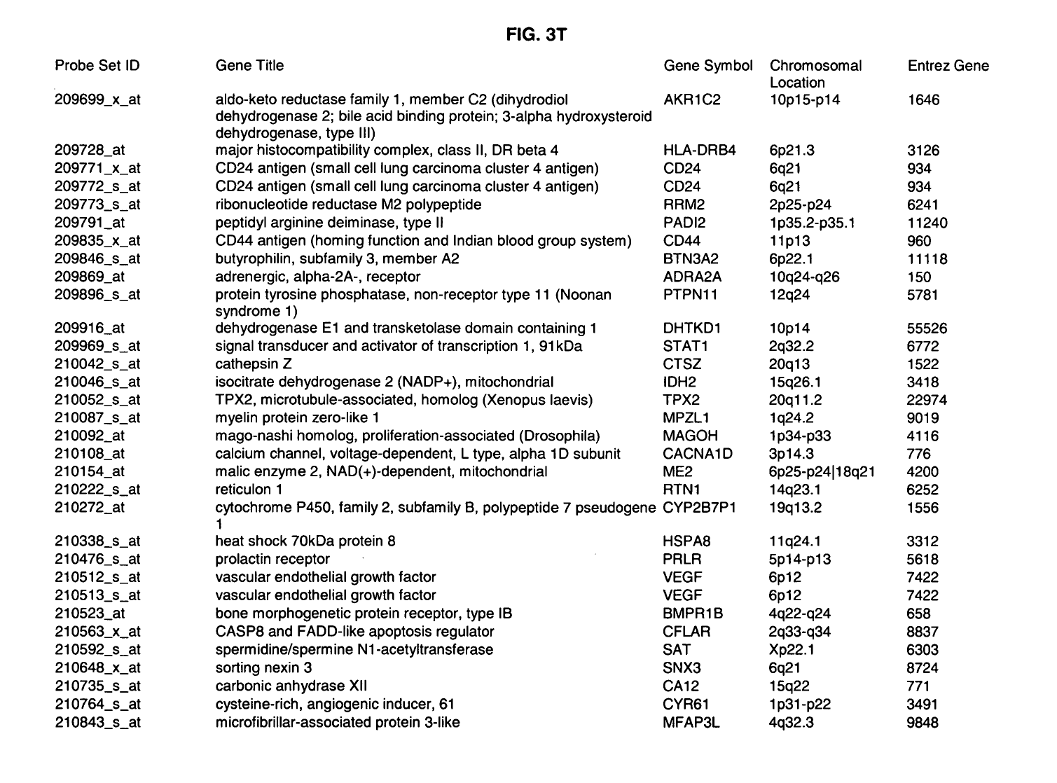

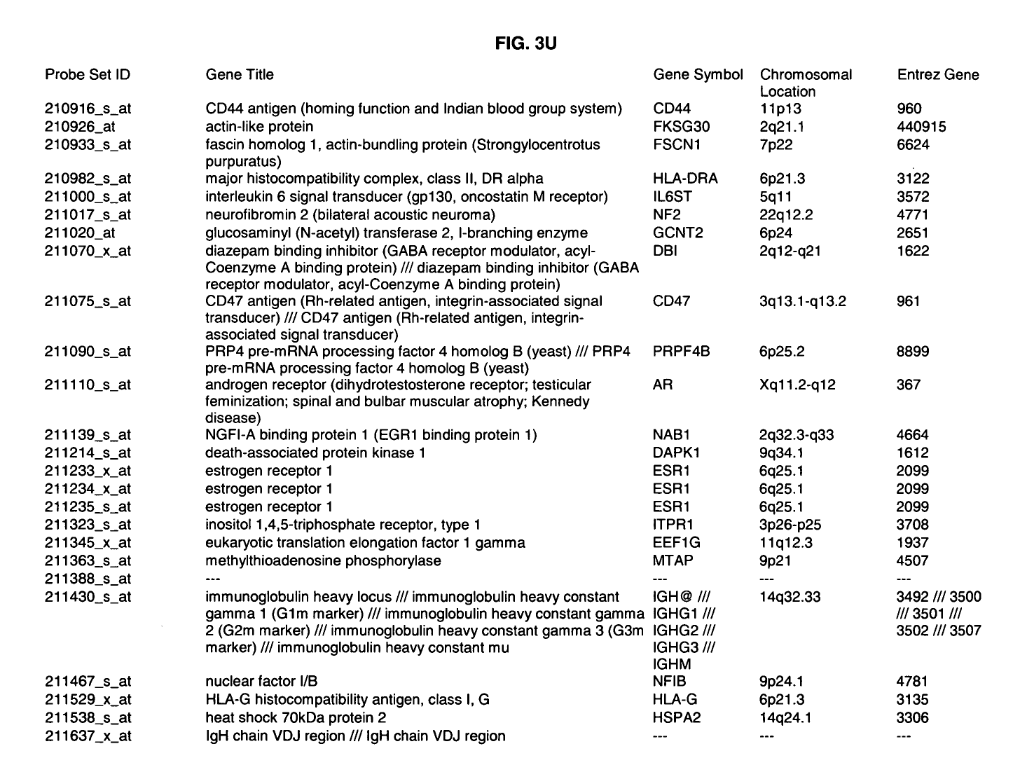

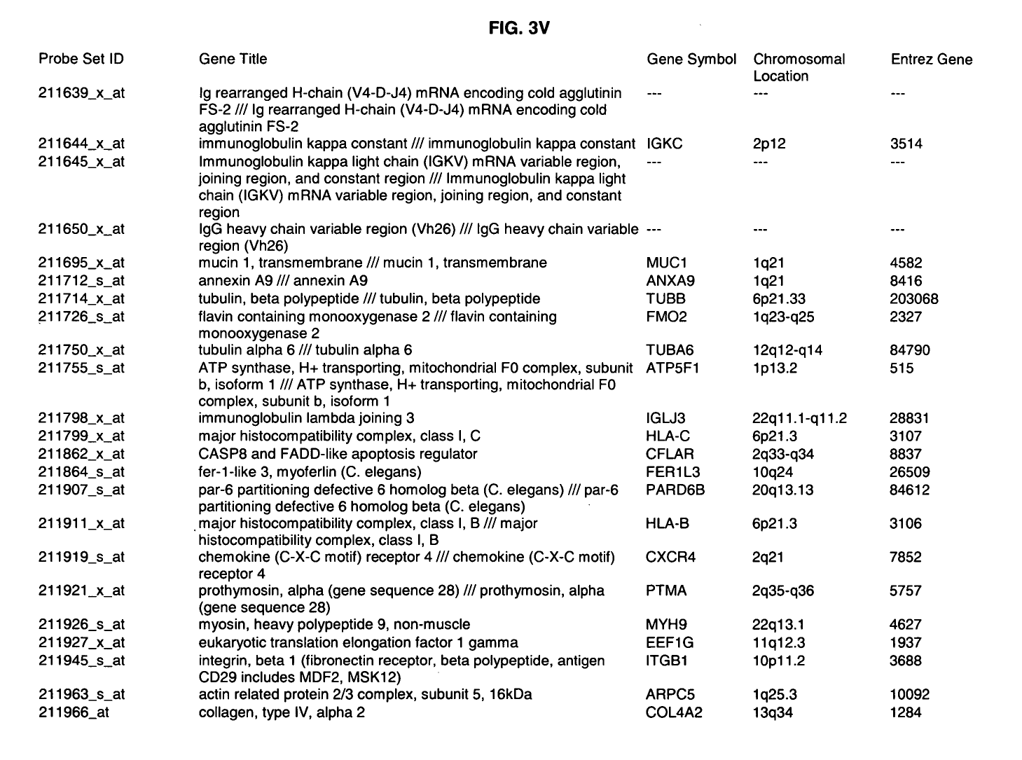

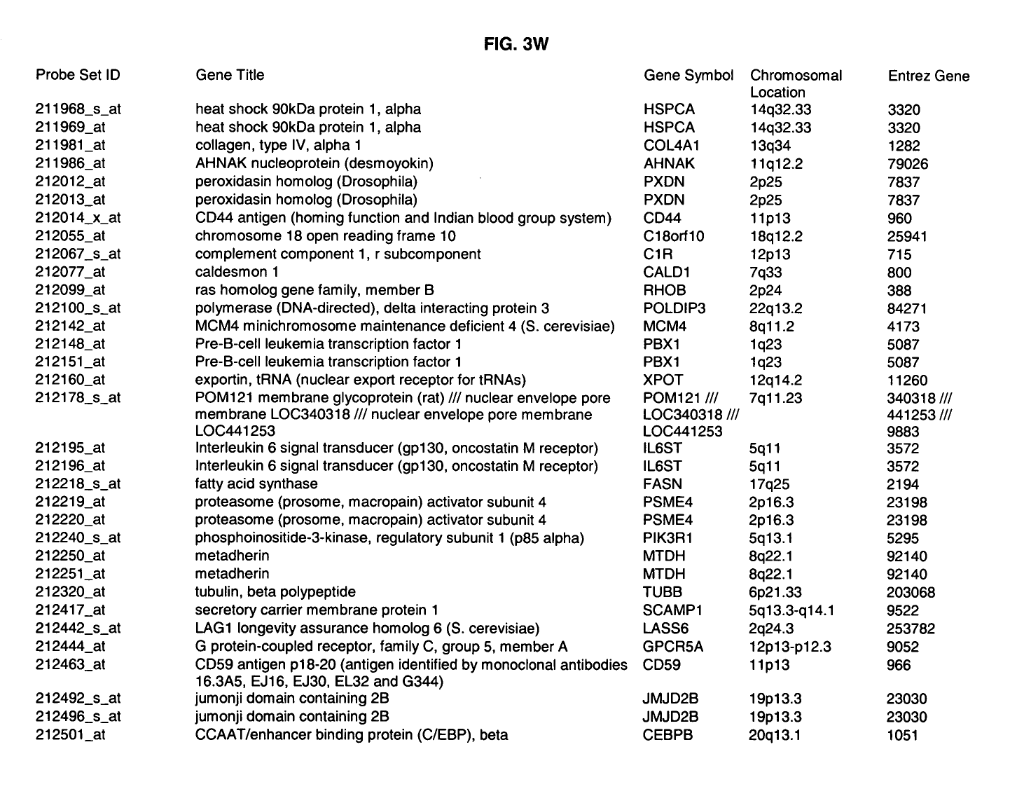

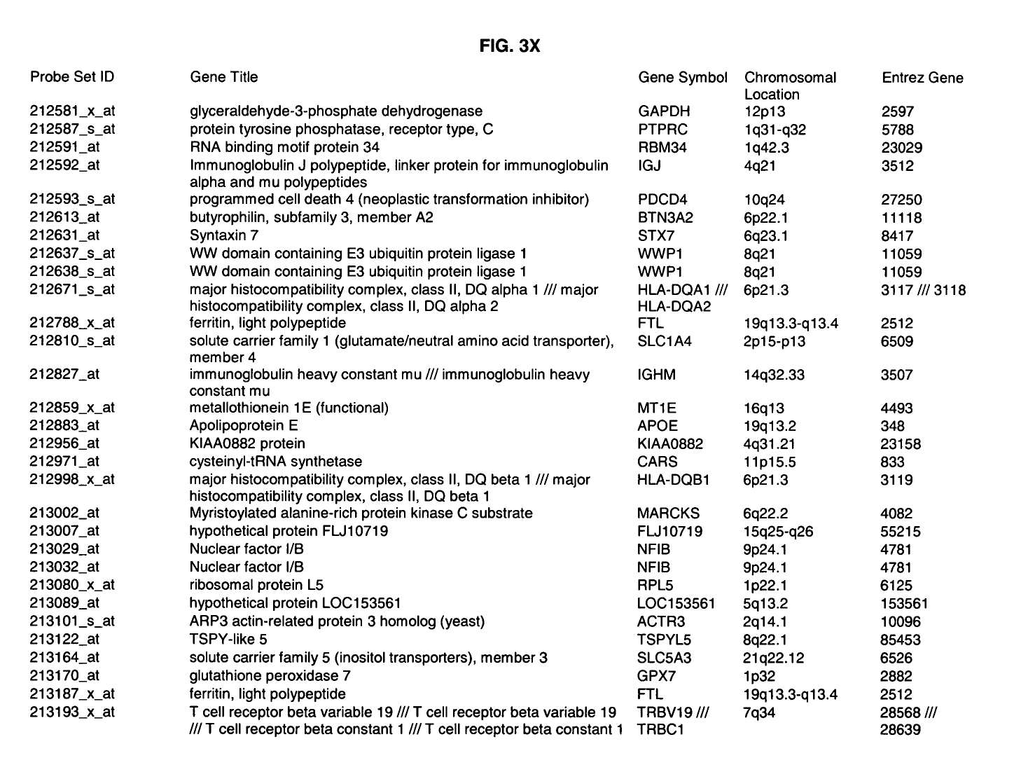

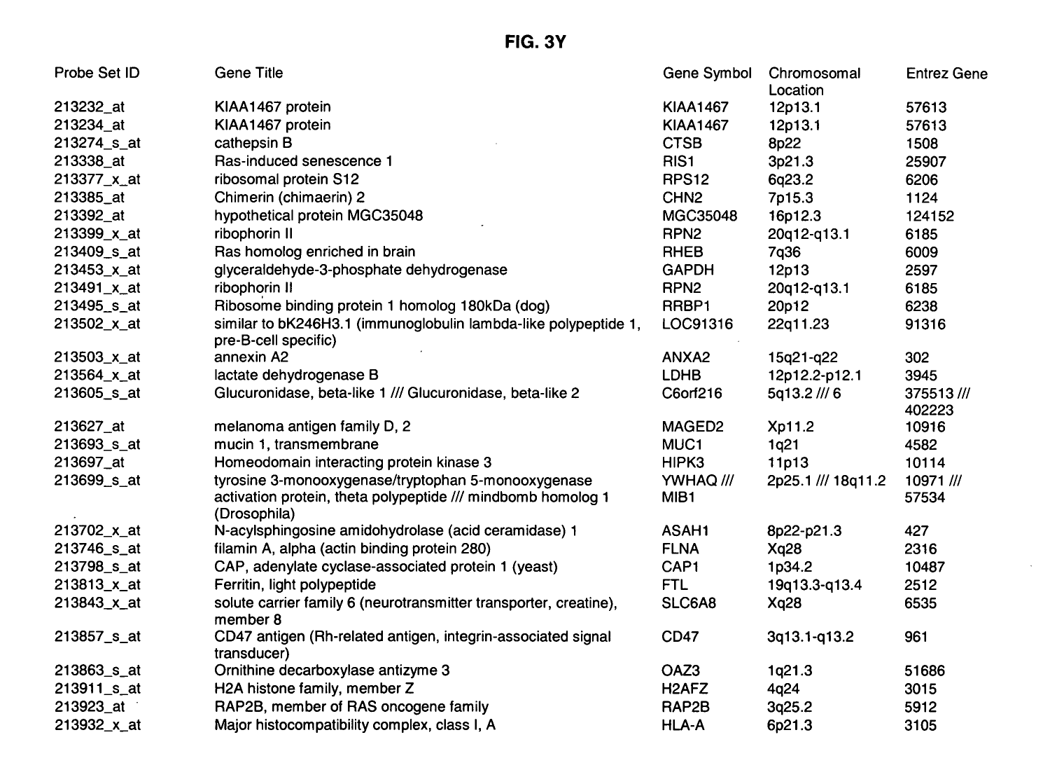

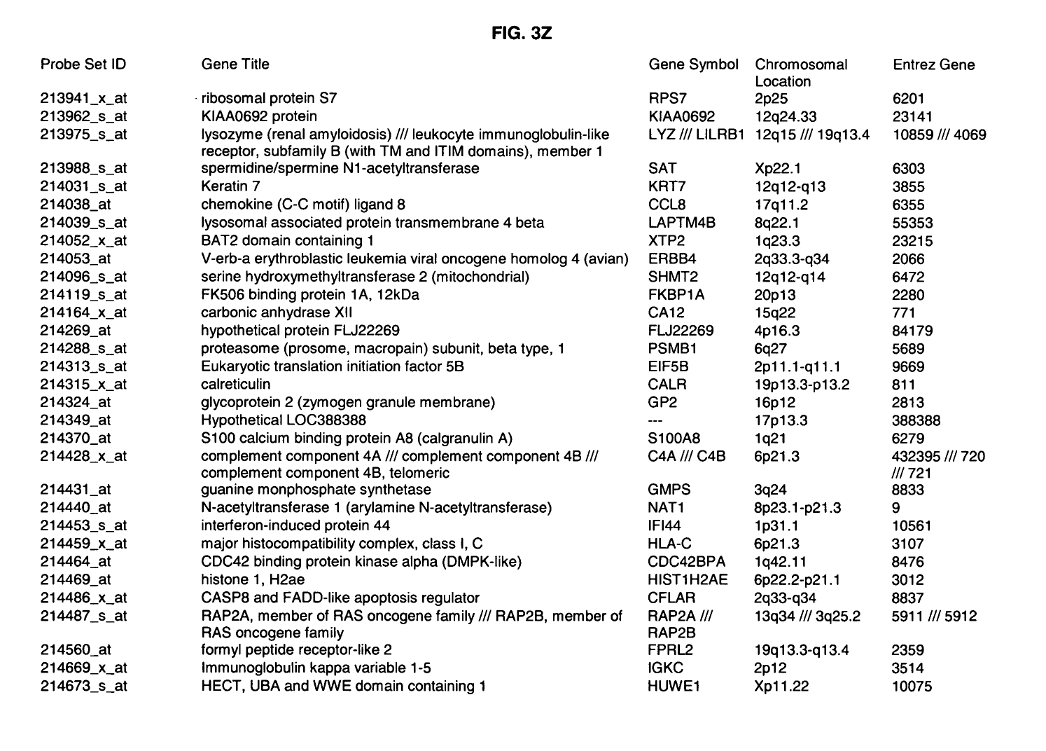

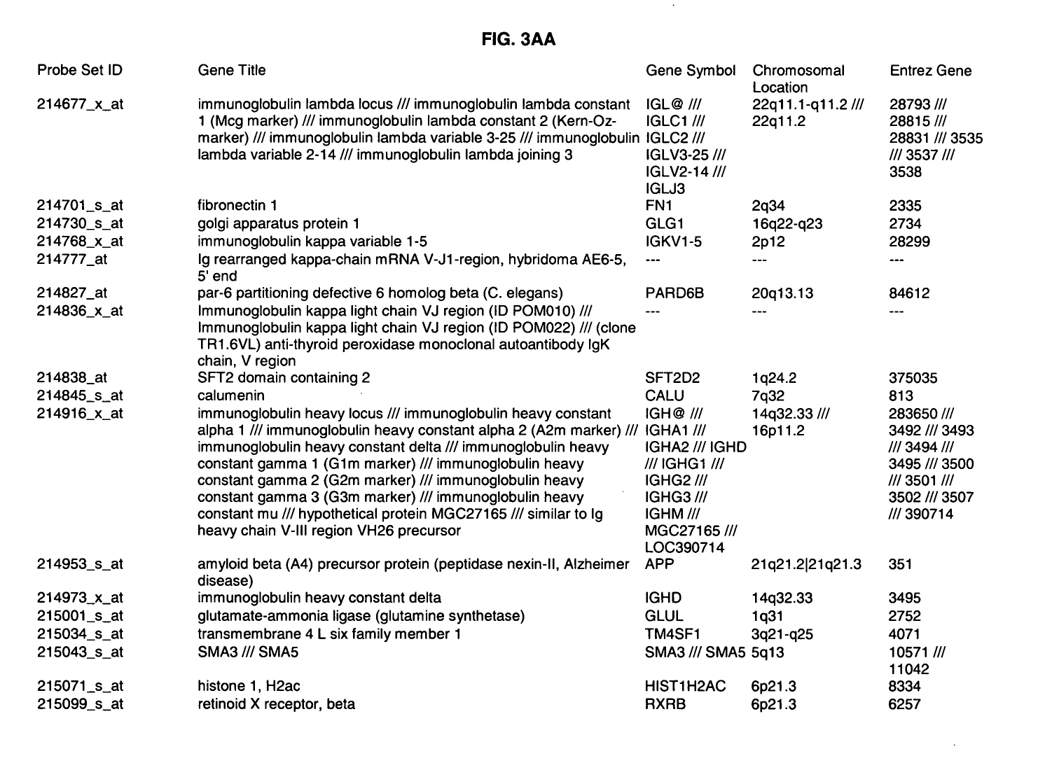

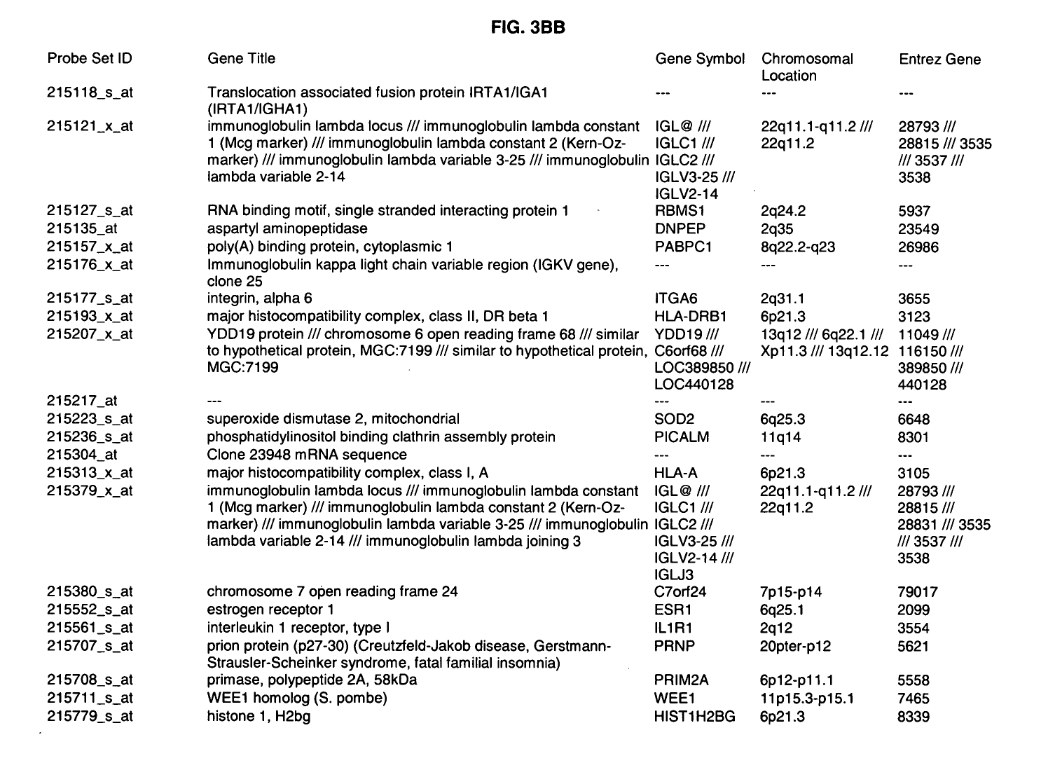

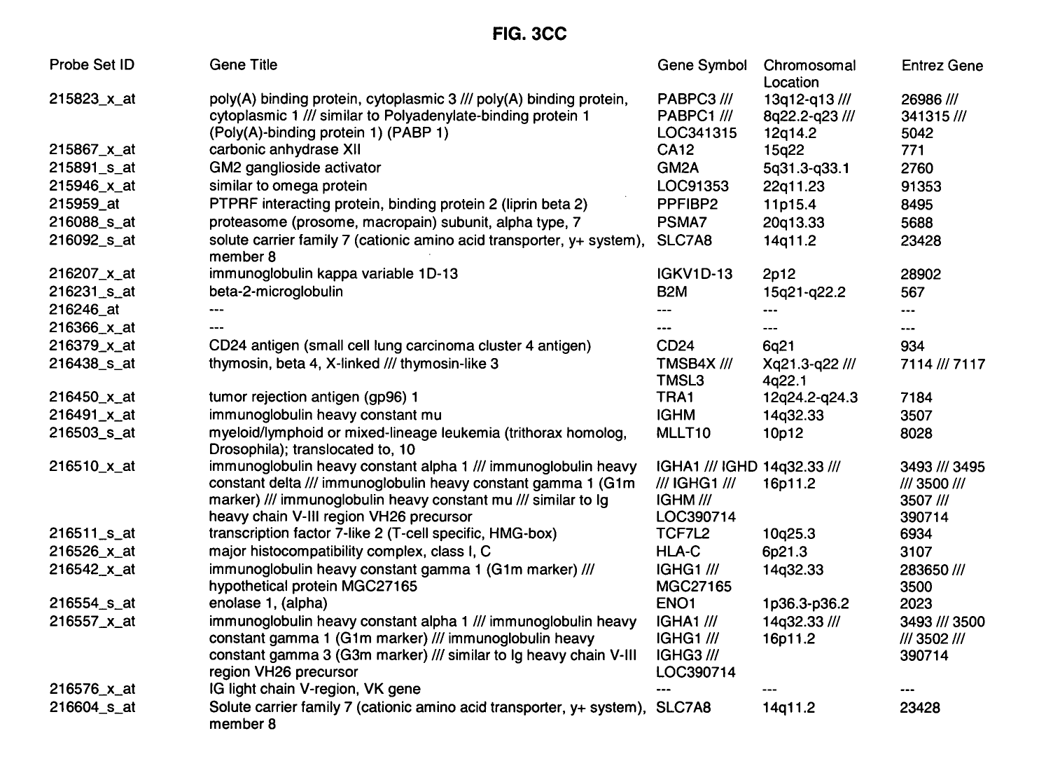

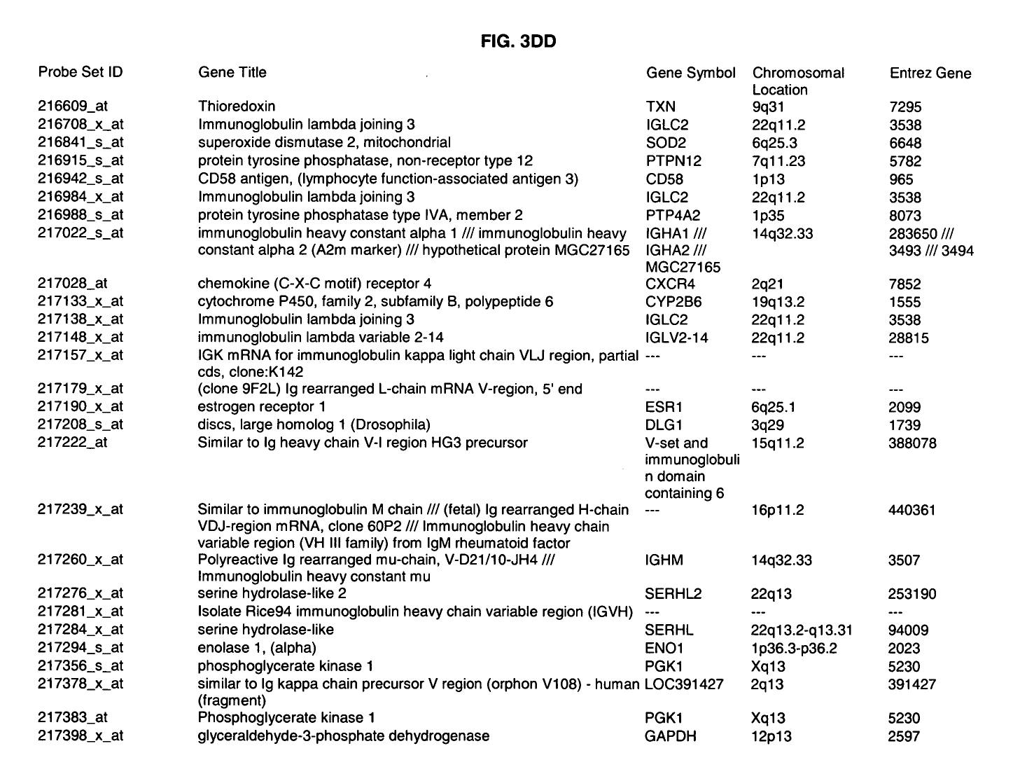

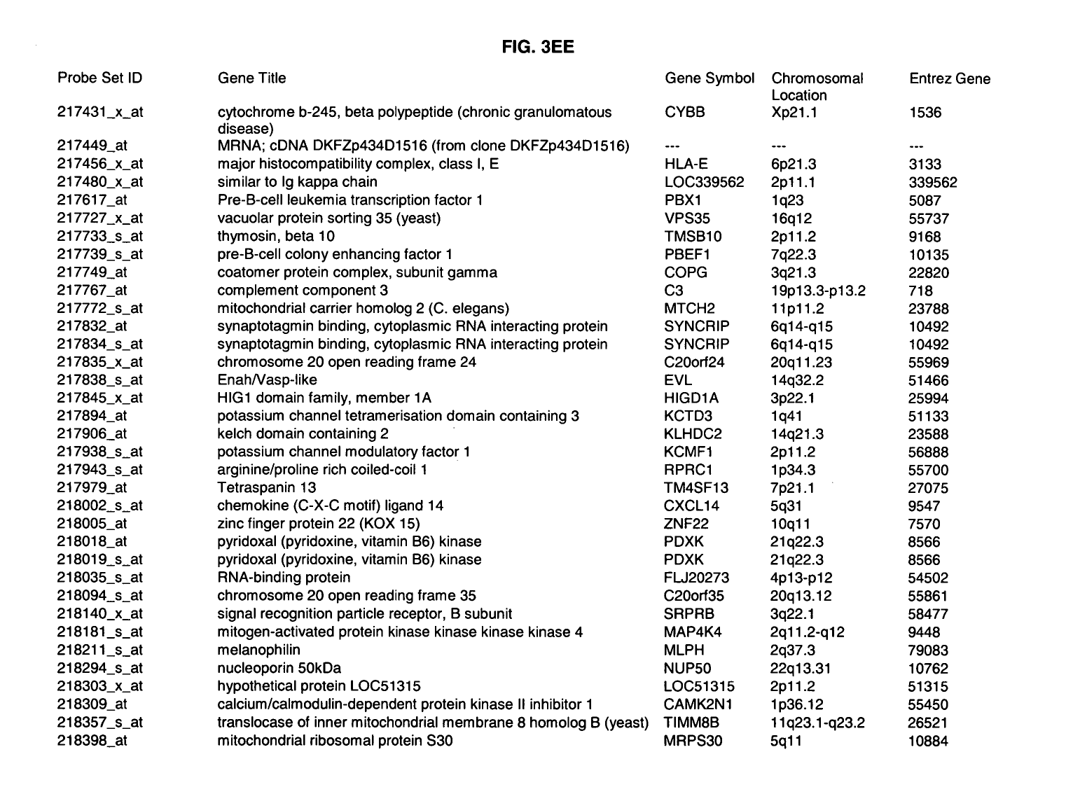

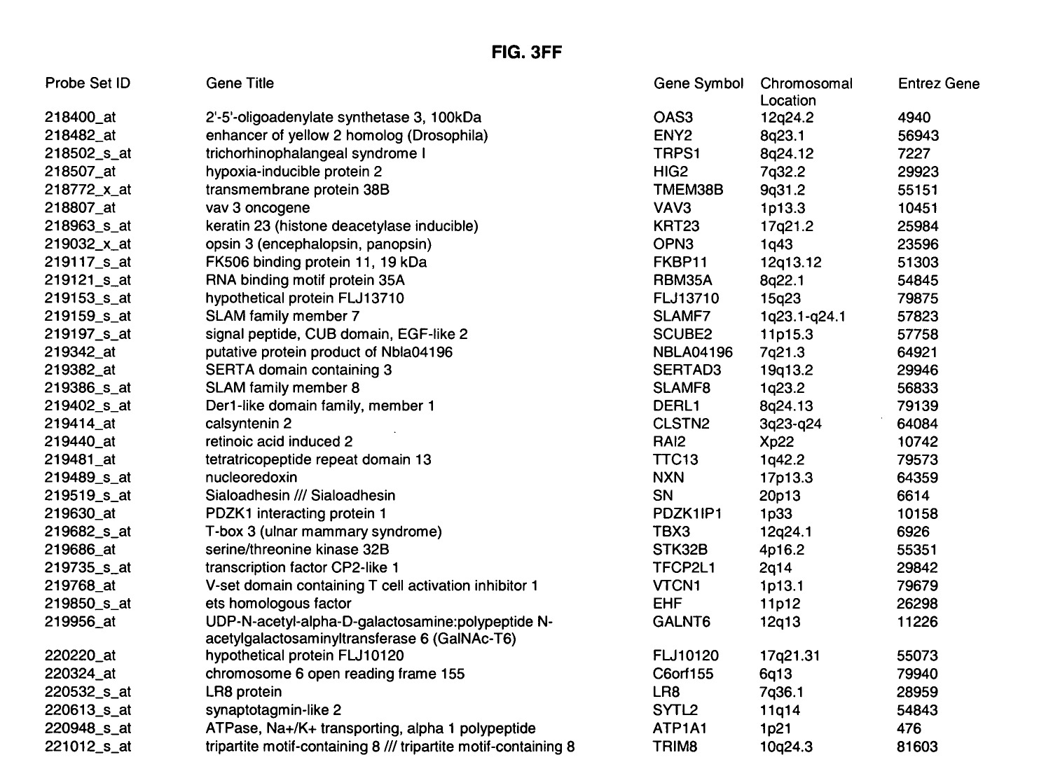

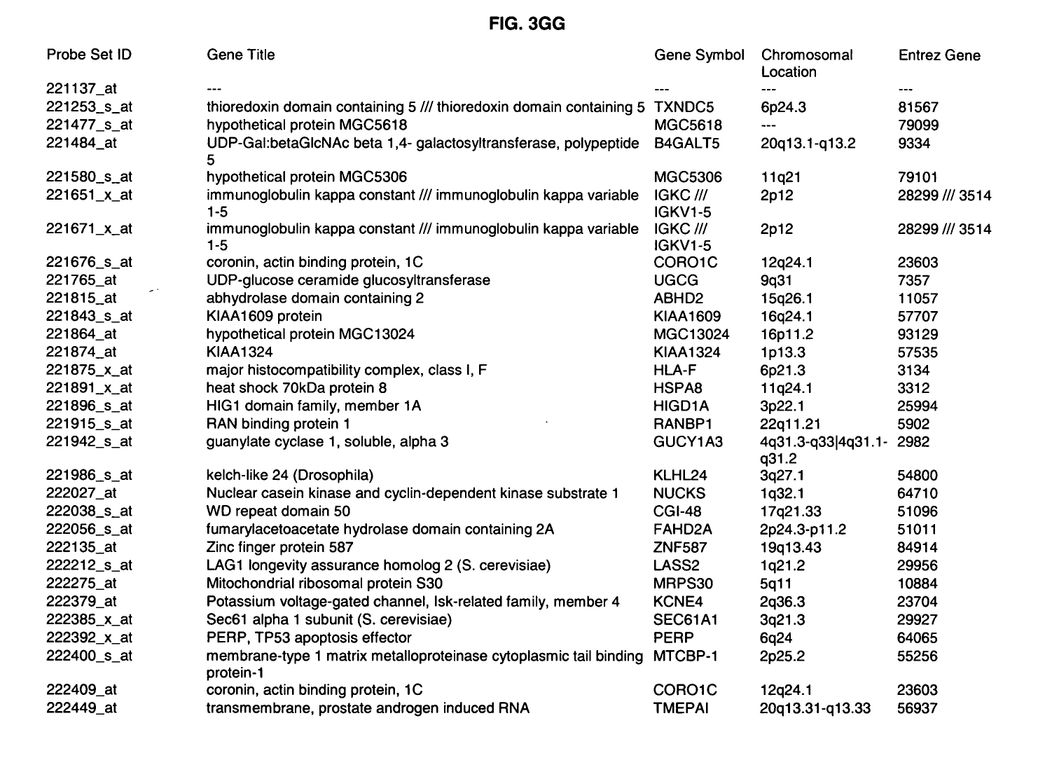

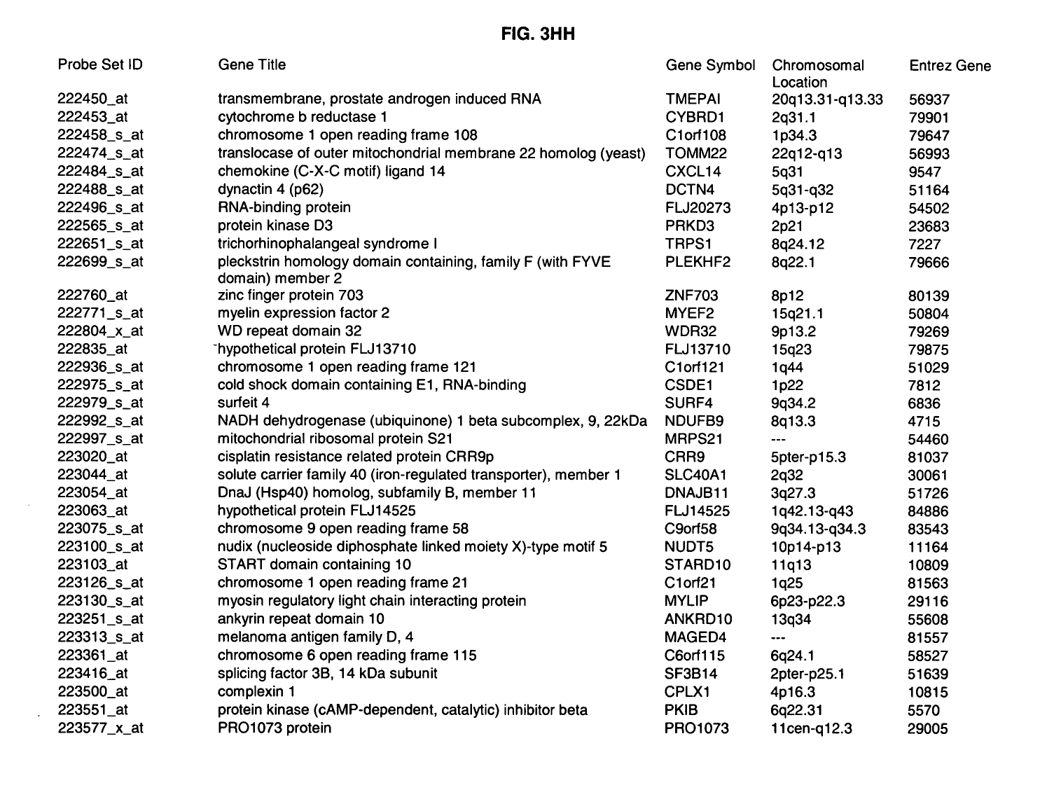

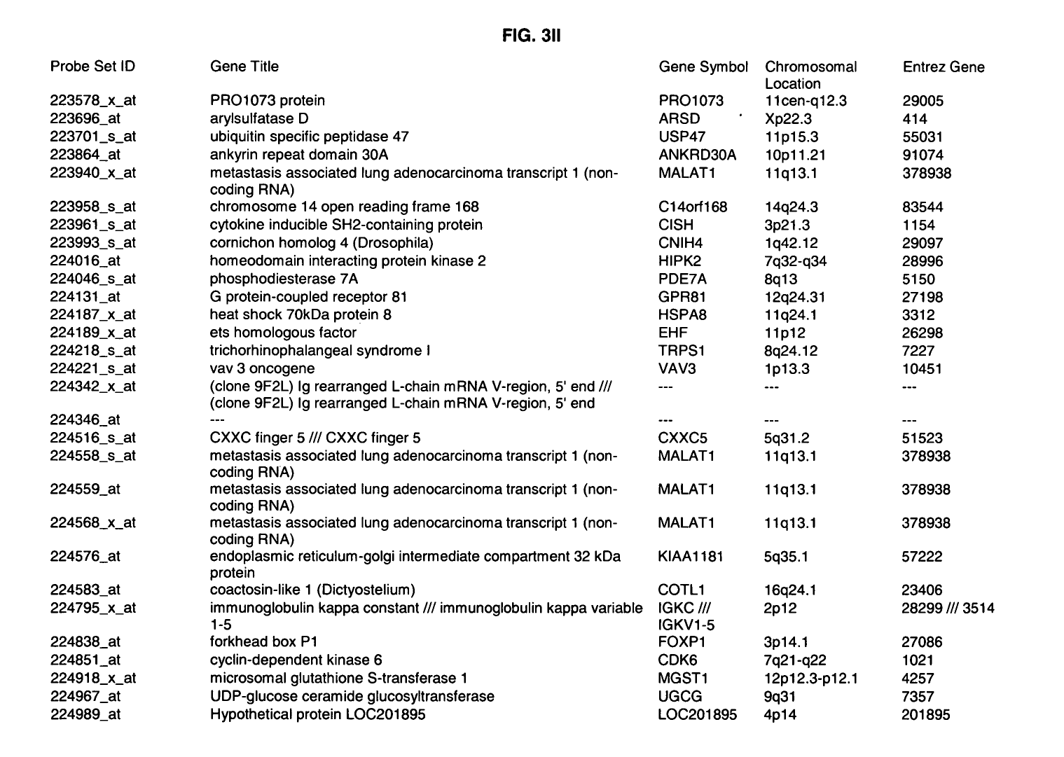

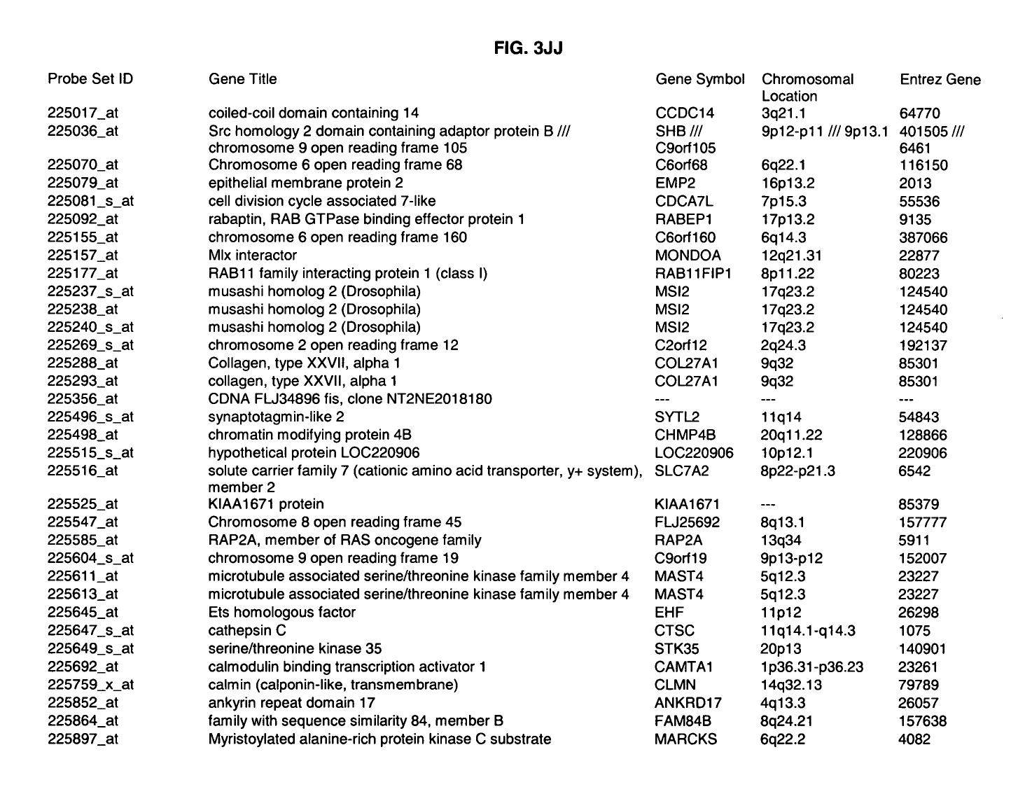

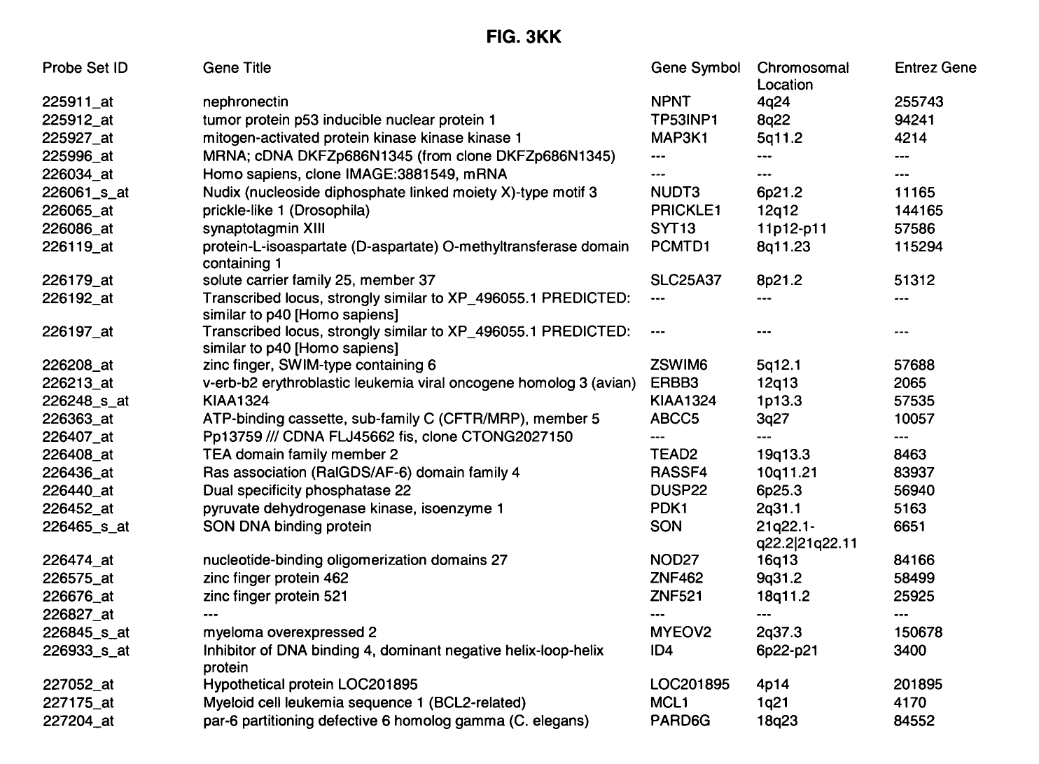

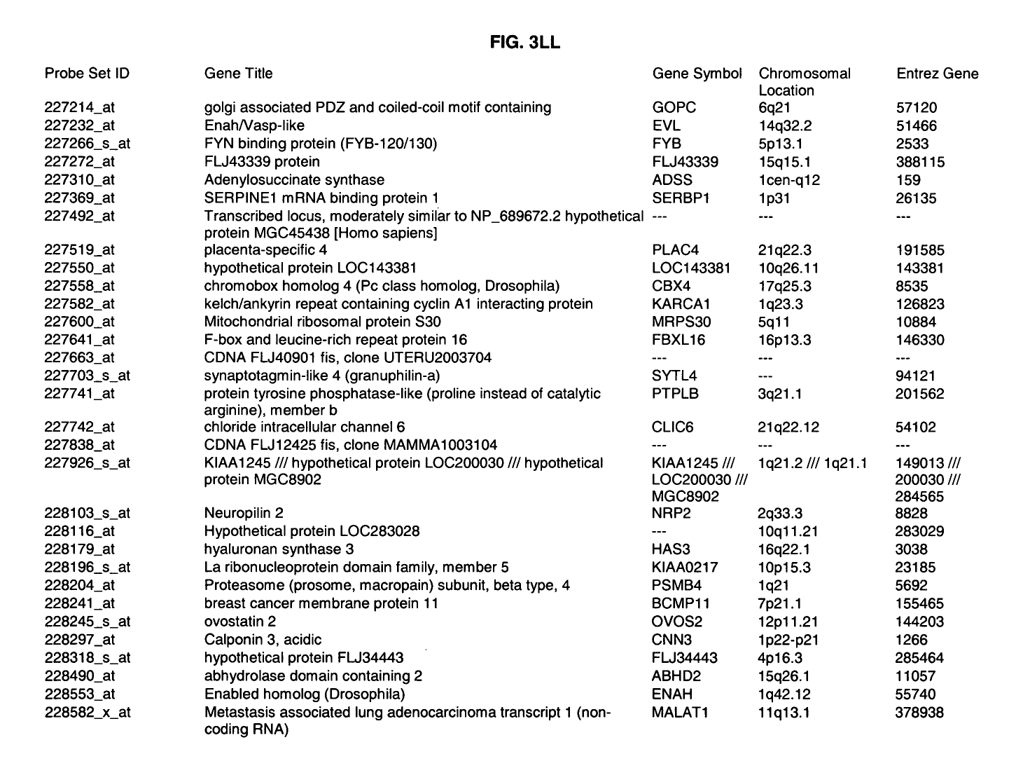

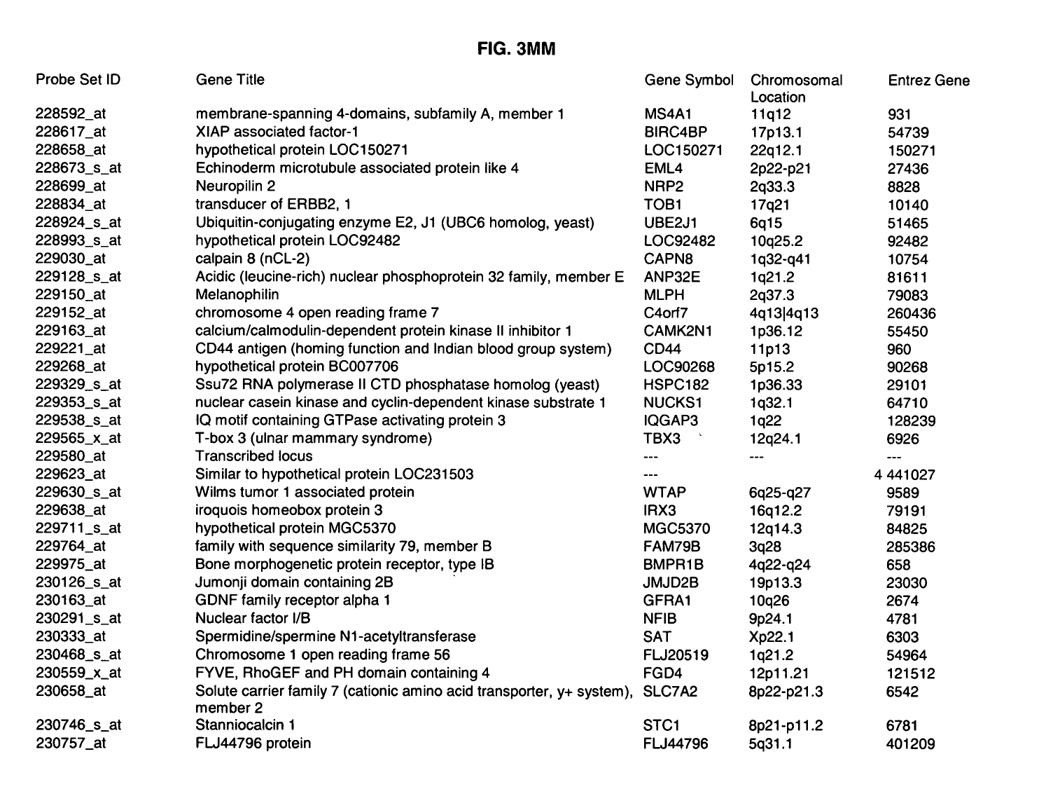

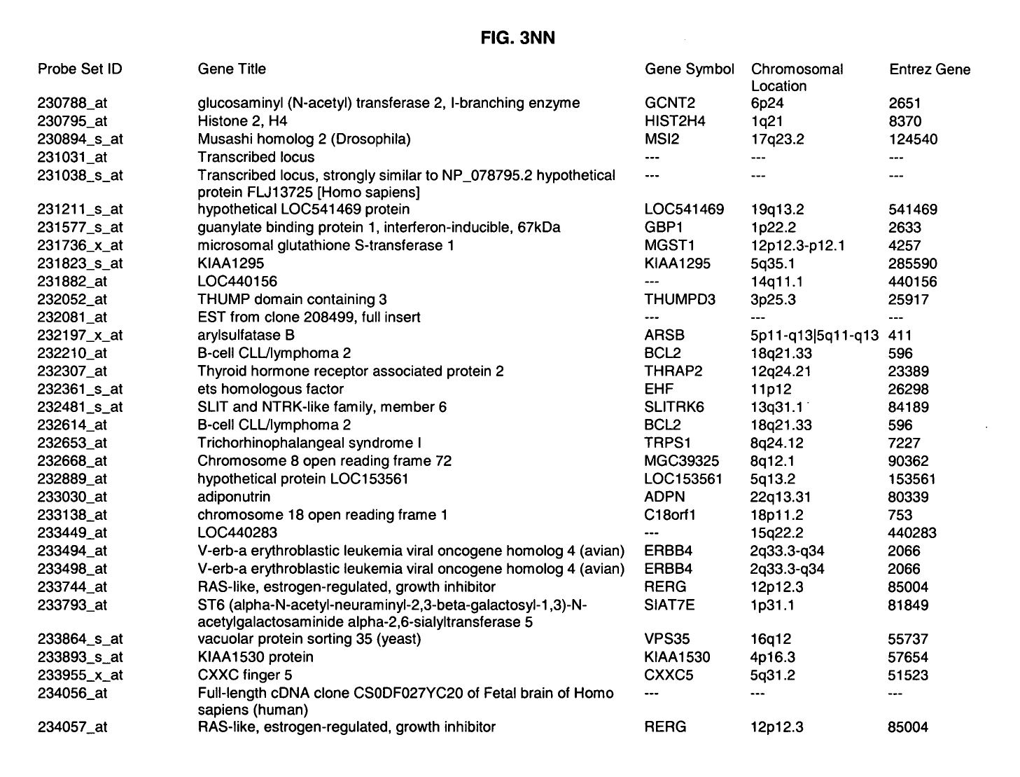

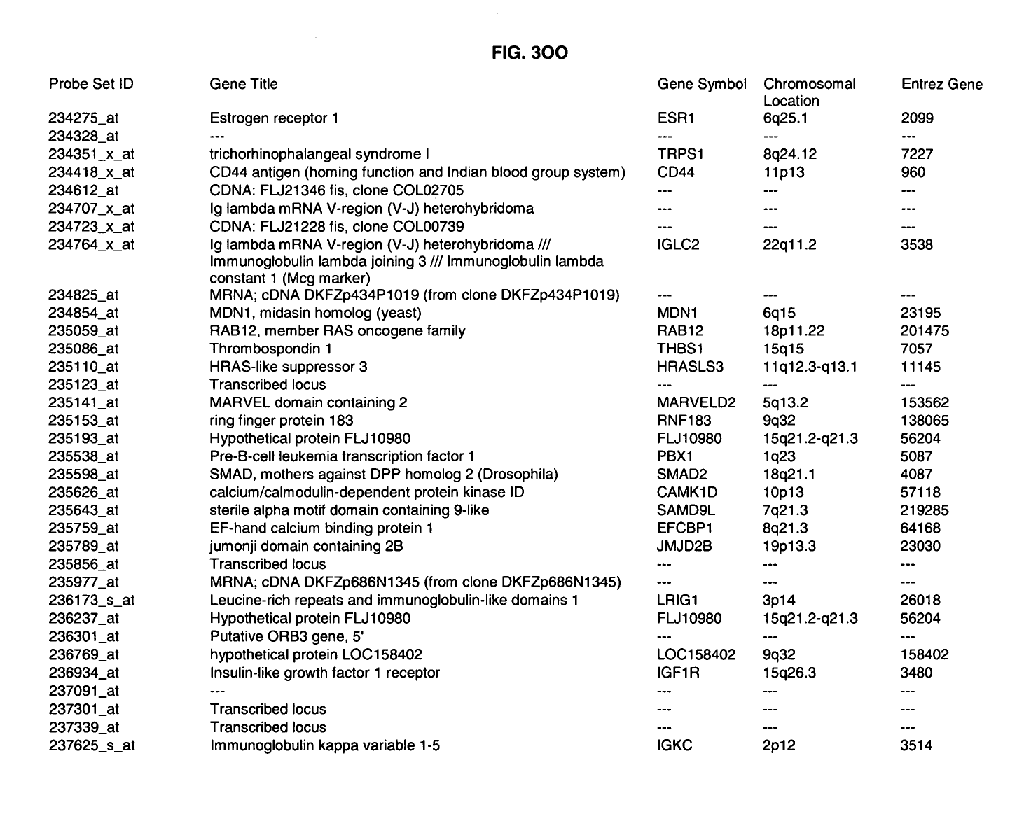

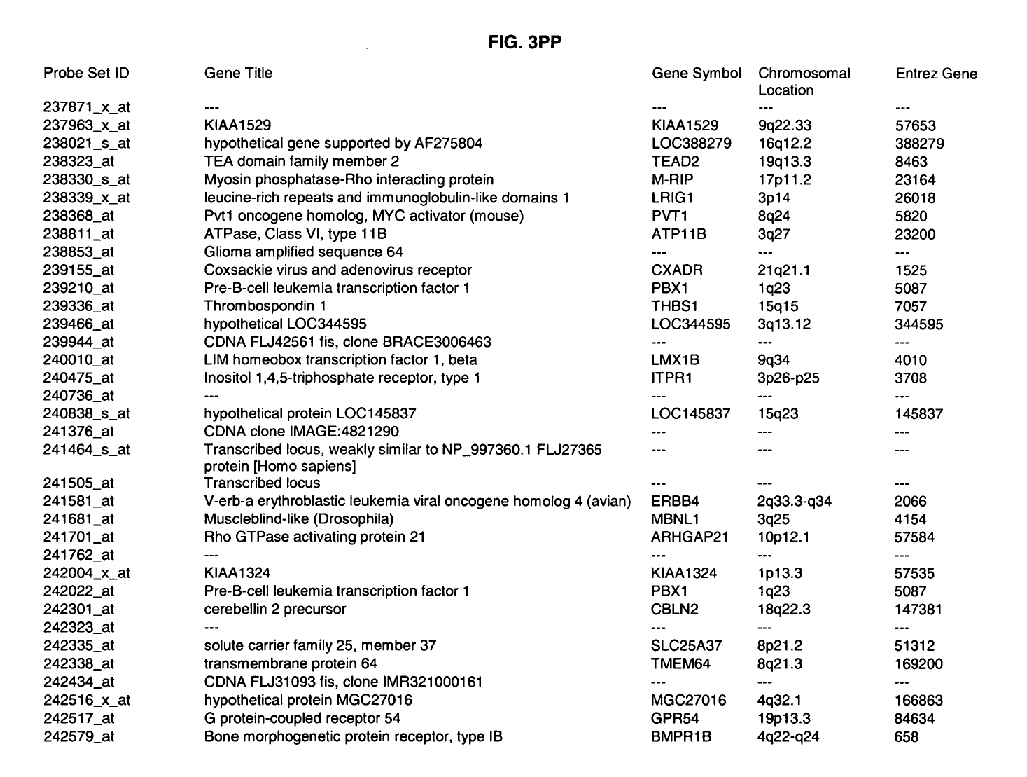

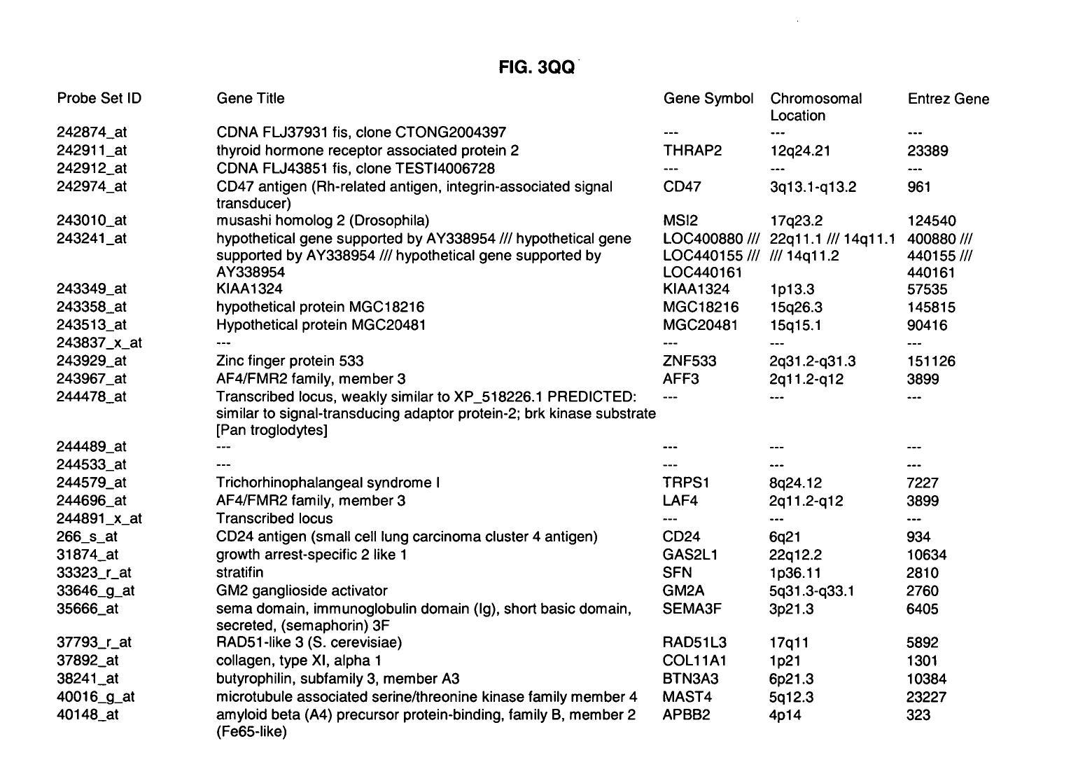

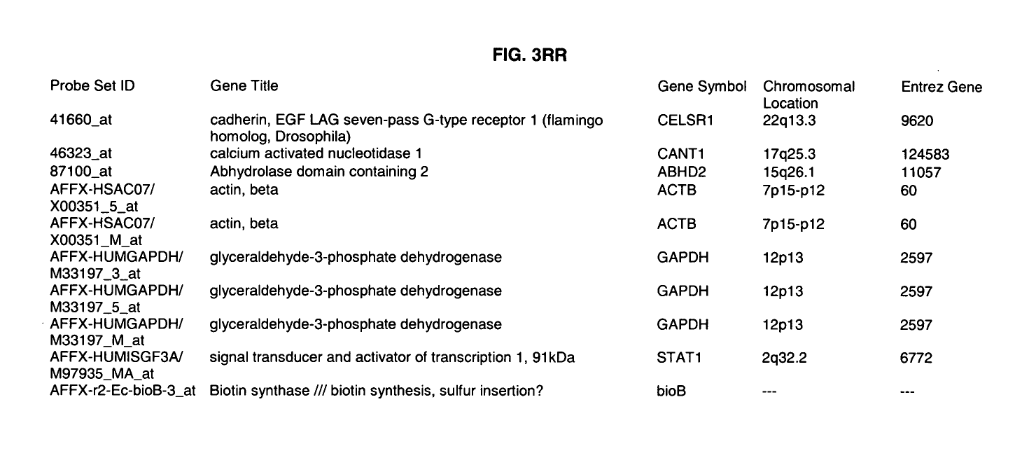

Gene expression information from 92 FFPET samples was analyzed using the methodology described in Example 1, above, using GeneChips® U133 Plus 2.0 and GeneChip® U133-X3P whole genome expression arrays (Affymetrix, Incorporated). The generated dataset was then identify over 900 differentially expressed genes between ER positive versus ER negative tumors using the SAM procedure, as described above. The list of differentially expressed genes included most known genes differentially expressed between ER positive versus ER negative tumors, such as ESR1, PR, GATA3, and SCUBE2. However, a number of previously unidentified differentially expressed genes were also identified. A complete listing of SAM positive genes identified using the GeneChip® U133 Plus 2.0 Array is shown in FIG. 2A through FIG. 2GG, and a complete listing of SAM positive genes identified using the GeneChip® U133-X3P shown in FIG. 3A through FIG. 3RR.

This dataset was also used to cluster the samples without supervision. This analysis found that the clustering is consistent with that previously published in the literature. For example, the gene expression data generated accurately clustered the breast cancer samples into ER positive and ER negative groups, and accurately clustered other gene expression groups previously published in the literature, including luminal, basal and HER2 positive groups.

All the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A method for analyzing gene expression levels from a formalin-fixed, paraffin-embedded tissue sample, comprising pre-hybridizing a labeled nucleic acid sample prepared from the formalin-fixed, paraffin-embedded tissue sample with a first microarray, hybridizing the unbound labeled nucleic acid sample with a second microarray, and detecting the labeled nucleic acid sample bound to the second microarray.

2. The method of claim 1, wherein the first microarray is a previously used microarray.

3. The method of claim 1, wherein the formalin-fixed, paraffin-embedded tissue sample comprises diseased tissue.

4. The method of claim 3, wherein the formalin-fixed, paraffin-embedded tissue sample comprises a tumor.

5. The method of claim 1, wherein the labeled nucleic acid sample is prepared from RNA isolated from the formalin-fixed, paraffin-embedded tissue sample.

6. The method of claim 5, wherein the RNA is isolated from a section prepared from the formalin-fixed, paraffin-embedded tissue sample.

7. The method of claim 6, wherein the section prepared from the formalin-fixed, paraffin-embedded tissue sample is between about 1 and about 10 microns thick.

8. The method of claim 3, wherein RNA is isolated from the diseased tissue in the formalin-fixed, paraffin-embedded tissue sample.

9. The method of claim 8, wherein the diseased tissue is identified by staining the formalin-fixed, paraffin-embedded tissue sample.

10. The method of claim 4, wherein RNA is isolated from the tumor in the formalin-fixed, paraffin-embedded tissue sample.

11. The method of claim 10, wherein the tumor in the formalin-fixed, paraffin-embedded tissue sample is identified by staining the formalin-fixed, paraffin-embedded tissue sample.

12. The method of claim 11, wherein the tumor in the formalin-fixed, paraffin-embedded tissue sample is identified by hematoxylin and eosin staining the formalin-fixed, paraffin-embedded tissue sample.

13. The method of claim 5, wherein the RNA isolated from the formalin-fixed, paraffin-embedded tissue sample is amplified.

14. The method of claim 13, wherein the RNA isolated from the formalin-fixed, paraffin-embedded tissue sample is converted into an amplified cDNA sample.

15. The method of claim 14, wherein the labeled nucleic acid sample is prepared by labeling the amplified cDNA sample.

16. The method of claim 15, wherein the amplified cDNA sample is labeled with BIO-ULS.

17. The method of claim 15, wherein the labeled amplified cDNA sample is purified.

18. The method of claim 17, wherein the purified labeled amplified cDNA sample is fragmented.

19. The method of claim 18, wherein the fragmented labeled amplified cDNA sample is purified subsequent to fragmentation.

20. A method for analyzing gene expression levels from a formalin-fixed, paraffin-embedded tissue sample, comprising identifying a disease area within the formalin-fixed, paraffin-embedded tissue sample, dissecting the identified disease area to obtain at least a first section of the diseased area, isolating RNA from the at least a first section of the diseased area, converting the RNA into an amplified cDNA sample, labeling the amplified cDNA sample, purifying the labeled cDNA sample, fragmenting the purified and labeled cDNA sample, purifying the fragmented cDNA sample, pre-hybridizing the fragmented cDNA sample with a first microarray, hybridizing the unbound fragmented cDNA sample with a second microarray, and detecting the fragmented cDNA sample bound to the second microarray.
